(19) United States
(12) Patent Application Publication
(10) Pub. No.: US 2023/0098646 A1
(43) Pub. Date: Mar. 30, 2023

(54) NEUROMODULATION DEVICES AND RELATED METHODS

(71) Applicants: Massachusetts Institute of Technology, Cambridge, MA (US); The Brigham and Women's Hospital, Inc., Boston, MA (US)

(72) Inventors: Robert S. Langer, Newton, MA (US); Polina Anikeeva, Lexington, MA (US); Carlo Giovanni Traverso, Newton, MA (US); Shriya Srinivasan, Cambridge, MA (US); Marc-Joseph Antonini, Boston, MA (US)

(73) Assignees: Massachusetts Institute of Technology, Cambridge, MA (US); The Brigham and Women's Hospital, Inc., Boston, MA (US)

(21) Appl. No.: 17/956,032

(22) Filed: Sep. 29, 2022

Related U.S. Application Data
(60) Provisional application No. 63/250,910, filed on Sep. 30, 2021.
(60) Provisional application No. 63/250,937, filed on Sep. 30, 2021.

(51) Int. Cl. A61N 1/36; A61B 5/0538; A61N 1/05; A61L 31/10; A61B 17/34; A61B 5/00

(52) U.S. Cl. CPC A61N 1/36007; A61B 5/0538; A61N 1/0509; A61L 31/10; A61B 17/3468; A61B 5/6851; A61B 17/3421; A61B 2560/063; A61B 2017/3454; A61B 2017/00867

(57) ABSTRACT

Articles and systems configured for treating GI motility disorders are generally provided. In some embodiments, an article comprising one or more electrodes (with both sensing and stimulating capabilities) may be configured to stimulate one or more tissues in the GI tract, electrically and/or chemically, to modulate peristalsis and/or allow neuromodulation. In some embodiments, a system comprises a controller that allows for close-loop operation of the article, e.g., such that the article may stimulate (e.g., via a feedback loop) the one or more organs in the GI tract upon receiving sensed parameters in the GI tract. In some embodiments, an implantation tool comprising a sensor may allow for submucosal or intramuscular implantation of an article. The implantation tool and the article may be useful for, for example, as a general platform for delivery of treating GI motility disorders and/or neuromodulation of the GI tract.

### RELATED APPLICATIONS

This application claims priority under 35 U.S.C. § 119(e) to U.S. Provisional Application No. 63/250,910, filed Sep. 30, 2021, entitled “NEUROMODULATION DEVICES AND RELATED METHODS,” and to U.S. Provisional Application No. 63/250,937, filed Sep. 30, 2021, entitled “NEUROMODULATION DEVICES AND RELATED METHODS,” each of which are incorporated herein by reference in their entirety for all purposes.

### GOVERNMENT SPONSORSHIP

This invention was made with government support under NS115025 awarded by the National Institutes of Health, and EEC1028725 and DMR1419807 awarded by the National Science Foundation. The government has certain rights in the invention.

### TECHNICAL FIELD

The present invention generally relates to system and articles including neuromodulation devices and related methods.

### BACKGROUND

GI motility disorders make up a significant number of patient cases seen in gastroenterology clinics. Pharmacological treatments and/or surgical approaches have been used to treat such GI motility disorders, but with limited success. Current electrical neuromuscular stimulation approaches lack mechanistic insights and devices suitable for implantation within the gastrointestinal (GI) tract. Some of the most significant challenges include a lack of closed-loop approaches for sensing and/or stimulation, coordinated activation of neuromuscular layers, and/or suitable implantation tools, etc.

Accordingly, improved articles, systems, and methods are needed.

### SUMMARY

The subject matter of the present invention involves, in some cases, interrelated products, alternative solutions to a particular problem, and/or a plurality of different uses of one or more systems and/or articles.

In one aspect, an article is provided. In some embodiments, the article comprises a polymeric component having an aspect ratio of at least 10:1; one or more electrodes disposed within the polymeric component, each electrode having a largest dimension aligned parallel to a largest dimension of the polymeric component; and one or more microfluidic channels disposed within the polymeric component, the microfluidic channel having a largest dimension aligned parallel to the largest dimension of the polymeric component.

In one aspect, an implantation tool is provided. In some embodiments, the implantation tool comprises a hollow needle comprising a tip, wherein the tip has a curved, grooved, pitchfork, broad, or dual point shape; an overtube associated with the hollow needle, wherein the overtube is adapted and designed to receive an article configured for submucosal implantation within a lumen of a subject; and an impedance sensor associated with the hollow needle, wherein the incision force of the hollow needle at an angle of 20 degrees relative to the lumen is less than or equal to 5 mN.

In another aspect, a device for performing neuromodulation is provided. In some embodiments, the device comprises an article and a controller in electrical communication with the article. In some embodiments, the article comprising a polymeric component having an aspect ratio of at least 10:1; one or more electrodes disposed within the polymeric component, each electrode having a largest dimension aligned parallel to a largest dimension of the polymeric component; and one or more microfluidic channels disposed within the polymeric component, the microfluidic channel having a largest dimension aligned parallel to the largest dimension of the polymeric component.

In yet another aspect, systems are provided. In some embodiments, a system comprises a controller configured to stimulate one or more electrodes upon sensing of a bolus of food and/or detecting a contractile and/or inhibitory event in gastrointestinal tract of a subject; an article configured for submucosal or intramuscular implantation, associated with the controller, comprising a polymeric component and the one or more electrodes, wherein the controller is configured to apply a voltage to the one or more electrodes upon sensing the bolus of food and/or detecting the contractile and/or inhibitory event.

In some embodiments, a system of neuromodulation comprises an article configured to stimulate one or more tissues associated with gastrointestinal tract in a subject using a plurality of electrodes and to induce production of metabolic hormones at a level that mimics a level of hormones produced in fed state in a subject, wherein the article comprises a polymeric component, the one or more electrodes disposed within the polymeric component, and a microfluidic channel disposed within the polymeric component.

In some aspects, methods are provided. In some embodiments, a method of submucosal or intramuscular implantation comprises inserting an incision needle at a lumen internal to a subject; localizing a submucosal or intramuscular layer based on a sensed parameter obtained from an impedance sensor associated with the incision needle; and via the incision needle, implanting an article in a submucosal or intramuscular layer of the lumen of the subject.

In some embodiments, a method for treating GI motility disorders comprises sensing a parameter adjacent a location internal a subject using one or more electrodes; and stimulating, via a controller associated with the one or more electrodes, electrically and/or chemically, an organ internal the subject when the sensed parameter reaches a threshold value; wherein the location and/or organ comprises gastrointestinal tract, wherein the parameter comprises measured impedance, pressure, electronystagmography (ENG), and/or electromyography (EMG), and wherein the controller operates in closed-loop with the one or more electrodes.

In some embodiments, a method for treating GI motility disorders comprises sensing, using a plurality of electrodes, a bolus of food present adjacent a location internal to a subject; and stimulating motility of an organ of the subject using the plurality of electrodes; wherein the location internal the subject and/or the organ comprises gastrointestinal tract.

In some embodiments, a method of neuromodulation comprises stimulating one or more tissues associated with gastrointestinal tract in a subject using a plurality of electrodes, wherein the step of stimulating the one or more tissues occurs in the absence of a bolus of food present in the gastrointestinal tract of the subject; and inducing production of metabolic hormones at a level that mimics a level of hormones produced in fed state in a subject.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C are schematic depictions of an article in comprising one or more electrodes in perspective view (FIG. 1A), cross-sectional view (FIG. 1B) and side view (FIG. 1C), in accordance with certain embodiments;

FIGS. 1D-1E are schematic depictions of various embodiments of the article shown in FIG. 1C, in accordance with certain embodiments;

FIG. 1F is a schematic drawing of an exemplary article, according to one set of embodiments;

FIG. 2 is a schematic depiction of a system comprising a controller, in accordance certain embodiments;

FIG. 3 is a flow chart depiction of a method for treating GI motility disorder, in accordance with certain embodiments;

FIGS. 4A-4C are schematic depictions of a method for treat GI motility disorders, in accordance with certain embodiments;

FIG. 5 is a flow chart depiction of a method of neuromodulation, in accordance with certain embodiments;

FIG. 6A is a schematic depiction of an implantation tool, in accordance with certain embodiments;

FIG. 6B is a schematic depiction of an implantation tool disposed in an endoscope channel, in accordance with certain embodiments;

FIGS. 7A-7F are schematic depictions of various types of needle tips, in accordance with certain embodiments;

FIGS. 8A-8D are schematic depictions of a method submucosal or intramuscular implantation, in accordance with certain embodiments;

FIG. 9A is a schematic depiction of a neuroprosthesis that augments esophageal and gastric motility through multichannel electric stimulation (ES) and chemical stimulation, in accordance with certain embodiments;

FIG. 9B is a schematic depiction of programmed patterns of ES and/or chemical stimulation that contract and inhibit the neuromusculature to recreate peristalsis upon sensing a bolus of food, in accordance with certain embodiments;

FIG. 9C is a schematic depiction of a submucosal implantation tool (SIT) that facilitates minimally invasive and precise implantation, in accordance with certain embodiments;

FIG. 10A is a schematic depiction of needle designs tested to optimize penetration force, in accordance with certain embodiments;

FIG. 10B shows a graph depicting force of penetration for various needle designs, in accordance with certain embodiments;

FIGS. 10C-10G show graphs of impedance measurements (FIG. 10G) as impedances were recorded as the needle advanced through the lumen (FIG. 10C), mucosa (FIG. 10D), muscularis propia (FIG. 10E) and peritoneum (FIG. 10F), in accordance with certain embodiments;

FIGS. 10H-10K show gross and histological images of the submucosal layer of esophagus (FIGS. 10H and 10J) and stomach (FIGS. 10I and 10K), respectively, via injection of saline that allowed accurate dissection of submucosal layers, in accordance with certain embodiments;

FIGS. 10L-10N show endoscopic visualizations aided the incision (FIG. 10L), implantation (FIG. 10M), and closure with one resolution clip (FIG. 10N), in accordance with certain embodiments;

FIGS. 11A-11C show fabrication and characterization of the closed-loop gastrointestinal neuroprosthesis, in accordance with certain embodiments, where FIG. 11A is a schematic depiction of the thermal drawing process during which a macroscopic preform (FIG. 11B) is heated and stretched into mm-scale fiber, and microelectrodes are fed into the preform and embedded into the final fibers (FIG. 11C);

FIG. 11D shows a graph of electrochemical impedance spectrum of the stainless-steel electrodes exposed through the polymer cladding using laser etching, in accordance with certain embodiments;

FIG. 11E shows a graph of cyclic voltammogram of the stainless-steel electrodes in 1× PBS, in accordance with certain embodiments;

FIG. 11F shows a graph of representative potential transient response to a +−4 mA symmetric, biphasic current pulse, in accordance with certain embodiments;

FIG. 11G shows a graph of E_{mc} of the electrode following a 0.5, 1, 2, 3, 4, 5, and 10 mA current pulse, in accordance with certain embodiments;

FIG. 11H shows a graph of accelerated aging of the electrode, in accordance with certain embodiments;

FIGS. 12A-12D illustrate optimization of electrical stimulation (ES) for esophageal and gastric motility, where ES parameters of amplitude (FIG. 12A), frequency (FIG. 12B), pulse train length (FIG. 12C), and amplitude (FIG. 12D) at 40 Hz and 0.5 seconds were optimized for esophageal motility using ex vivo and in vivo models, in accordance with certain embodiments;

FIGS. 12E-12H show images illustrating the percentage of closure in the esophageal lumen, in response various esophageal muscle activation, according to some embodiments;

FIGS. 12I-12J show images of the opening (FIG. 12I) and closing (FIG. 12J) of the pylorus under ES was utilized to characterize gastric motility rate, in accordance with certain embodiments;

FIGS. 12K-12L show data illustrating a panel of ES parameters (FIG. 12K) tested for stomach motility (FIG. 12L), where all stimulation conditions yielded an increase in peristaltic rate, with C optimizing activity for the most minimal ES parameters, in accordance with certain embodiments;

FIG. 13A is a schematic representation of a controller scheme of the closed-loop actuation of the GI tract using the neuroprosthesis, according to some embodiments, where the controller parameters are calibrated on a given disease;

FIG. 13B shows a graph of impedance amplitude at 500 Hz, 1000 Hz, and 2000 Hz versus bolus force, according to some embodiments;

FIG. 13C shows a graph of EMG of the stomach upon stimulation by the neuroprosthesis, according to certain embodiments;

FIG. 13D shows a representative intraluminal manometry measurement of the esophagus during a reflex-initiated swallow and neuroprosthesis-actuated contraction, in accordance with some embodiments;

FIG. 13E shows a graph of neuroprosthesis-actuation peristalsis in the esophagus that demonstrates a coordinated swallowing pattern, in according to some embodiments;

FIG. 13F shows a graph of filtered EMG and raw signal recorded at each electrode during neuroprosthesis-initiated peristalsis in the esophagus, in accordance with certain embodiments;

FIG. 13G shows a set of images illustrating endoscopic visualization of the bolus before (top), during (middle) and after (bottom) contraction of the esophageal muscle enable bolus propagation, according to some embodiments;

FIG. 13H shows a representative intraluminal manometry of the esophagus at rest demonstrating a tonic LES (left) and glucagon infusion by the neuroprosthesis yields relaxation of this LES (right), in accordance with certain embodiments;

FIG. 13I shows a graph of temporary relaxation of the LES induced by glucagon infusion, in accordance with certain embodiments;

FIG. 13J shows a graph that demonstrates a continuous stimulation yielding less than 20% decrease in force production over a 200 second trial, in accordance with certain embodiments;

FIG. 13K is a graph of representative manometry during antero- and retrograde peristalsis, in accordance with certain embodiments;

FIG. 14A is a schematic representation illustrating relationship of afferent mechanotransduction driven by motility to metabolic secretions, in accordance with certain embodiments;

FIGS. 14B-14F show graphs depicting response of various hormones in the blood in response to stimulation between minute 30-60 normalized to their baseline levels in animals with no stimulation, esophageal, and gastric stimulation (n=5), in accordance with certain embodiments;

FIG. 15A-15D show images of components of the submucosal implant tool: overtube (FIG. 15A), incision needle (FIG. 15B), localization guide wire (FIG. 15C), and prosthesis (FIG. 15D) through overtube, in accordance with certain embodiments;

FIG. 16 shows an image visualizing the implantation of the neuroprosthesis in the stomach upon explant, in accordance with certain embodiments;

FIGS. 17A-17D show SEM images of a smooth surface of the neuroprosthesis (FIG. 17A) lining the tissue, sideview of the neuroprosthesis (FIG. 17B) where the etch has been performed, etched surface of the neuroprosthesis (FIG. 17C) where the electrode is exposed, and microscopic image of the etched surface of the neuroprosthesis (FIG. 17D), in accordance with certain embodiments;

FIGS. 18A-18C show graphs of voltage transient in response to a biphasic symmetric current pulse with interphase delay (100 μsec half-phase period, 33.3 μsec interphase delay) (FIG. 18A), voltage transient in response to accelerated aging with continuous biphasic symmetric current pulse (4 mA, 100 μsec half-phase period, 33.3 μsec interphase delay) (FIG. 18B), and cyclic voltammogram before and after the accelerated aging experiment (144M pulses) (FIG. 18C), in accordance with certain embodiments;

FIGS. 19A-19B show fluoroscopy images of a barium pellet before (FIG. 19A) and after contraction (FIG. 19B) of the esophagus, in accordance with certain embodiments;

FIG. 20 shows a graph of peristaltic rate that illustrates a significant stomach motility increased as a result of neuroprosthetic stimulation, in accordance with certain embodiments;

FIG. 21A shows a graph of an extract exposure test that demonstrates no significant difference between negative controls and the neuroprosthesis at 24 and 168 hours, in accordance with certain embodiments;

FIGS. 21B-21H show images illustrating swine esophagus after 7-day implantation in (FIG. 21B), a fibrous capsule formed around the implant site with no significant inflammation or adverse reaction (FIGS. 21C and 21E) compared to control sites (FIGS. 21D and 21F), and site of esophageal (FIG. 21G) and stomach (FIG. 21H) implantation, in accordance with certain embodiments;

FIG. 22 shows an image of an octagonal rotary jig, in accordance with certain embodiments; and

FIGS. 23A-23B show images of a study of spatiotemporal dynamics of peristalsis in an ex vivo tissue maintenance system using commercial needle electrodes spaced at specified lengths and depths in the tissue, in accordance with certain embodiments.

### DETAILED DESCRIPTION

Articles and systems configured for treating GI motility disorders are generally provided. Certain embodiments comprise an article comprising one or more electrodes and optionally one or more microfluidic channels, e.g., for stimulating one or more tissues in the GI tract, electrically and/or chemically, in the presence of a sensed food stimuli. In some embodiments, in the absence of a food stimuli, a system comprising the article may be employed for neuromodulation, e.g., to induce an illusory state of satiety in a subject by inducing production of metabolic hormones at a level that mimics a level of hormones produced in fed state in a subject. In some embodiments, a system comprises a controller that allows for closed-loop operation of the article, e.g., such that the article may stimulate (e.g., via a feedback loop) the one or more organs in the GI tract upon receiving sensed parameters (e.g., food stimuli) in the GI tract. Certain embodiments comprise an implantation tool comprising a sensor (e.g., an impedance sensor), e.g., for submucosal or intramuscular implantation of the article. The implantation tool and the article may be useful for, for example, as a general platform for delivery of treating GI motility disorders and/or neuromodulation within the GI tract.

There has been a general lack of effective and targeted neuromuscular stimulation strategies for treating GI motility disorders. For example, current electrical stimulation (ES) devices typically operate in an open-loop fashion, lacking the ability to sense a food stimuli and stimulate the GI tract based on the sensed stimuli in a coordinated fashion. Furthermore, there is a lack of ES devices that are capable of modulating both contractile and inhibitory neuromuscular activity, thereby limiting the type of GI motility disorders that can be treated. Moreover, there is a lack of minimally invasive strategies for implanting ES articles and/or system for treating GI motility disorders.

In view of the above, the Inventors have recognized a need for the development of controlled and targeted neuromuscular stimulation strategies, closed-loop sensing and stimulation capabilities, and minimally invasive implantation strategies. Accordingly, the development of an implantable article comprising one or more electrodes and/or microfluidic channels containing chemicals may allow for controlled and targeted electrochemical stimulation of the GI tract. Advantageously, the articles described herein may have both sensing and stimulation capabilities, and may allow for closed-loop and disease-specific electrochemical stimulation of the GI tract in the presence of food stimuli. Furthermore, the article may allow for controlled neuromodulation via stimulation of one or more tissues within the GI tract, e.g., to mimic metabolic responses of a fed state in the absence of a food stimuli. The article may allow, in some cases, for modulation of both contractile and inhibitory neuromuscular activities, and e.g., thus may be employed to treat various types of GI disorders. Additionally, or alternatively, the development a minimally invasive implantation tool for submucosal or intramuscular implantation may advantageously allow for accurate localization of the submucosal or intramuscular layer. The implantation may have certain features (e.g., needle design, hook, sensor, etc.) that imparts it with a set of favorable properties, e.g., such as low-force incision at a target location, compatibility with typical endoscopes and endoscopic procedures, etc.

The term “subject,” as used herein, refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the article and/or the actuating component.

The present disclosure is generally related to articles and devices configured for implantation (e.g., submucosal or intramuscular implantation) to a location in a subject, e.g., to treat GI motility disorder in the subject.

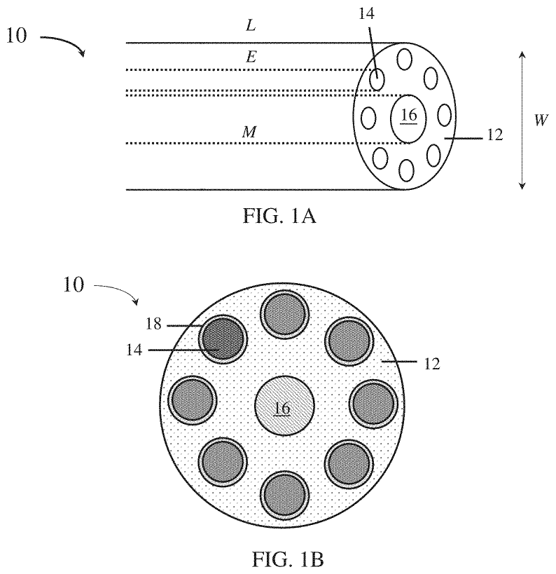

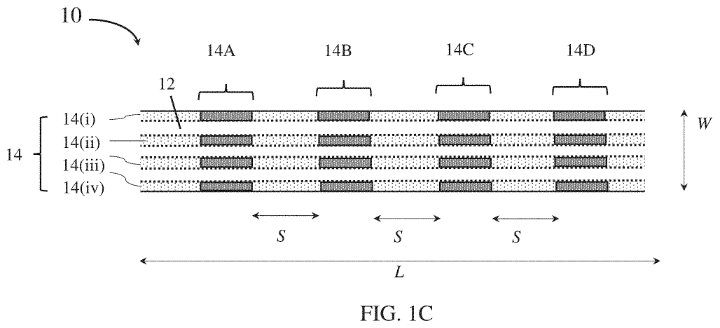

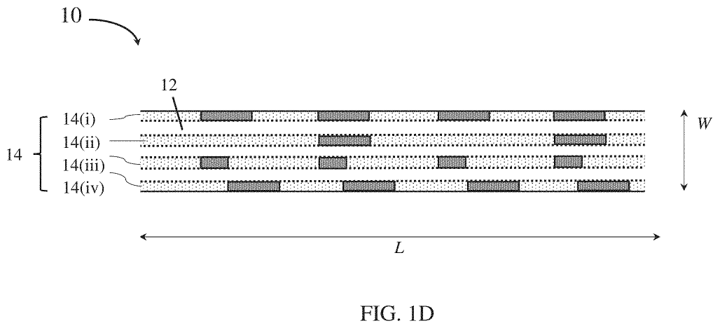

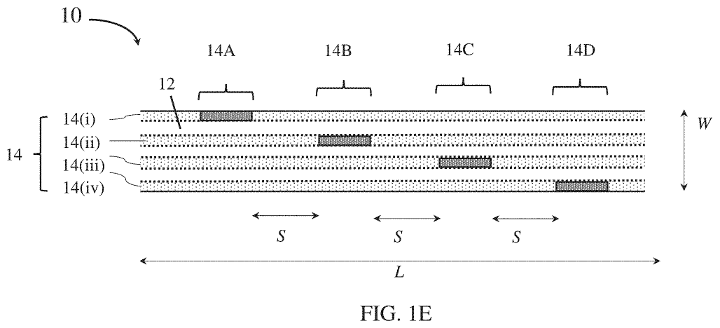

In some embodiments, an article configured to treat GI motility disorders is described herein. In some embodiments, the article comprises a polymeric component and one or more electrodes disposed within (e.g., embedded in) the polymeric component. In one set of embodiments, the article comprise a plurality of electrodes (e.g., at least two electrodes) disposed within the polymeric component. A non-limiting representation of one such embodiment is shown in FIGS. 1A-1C. As shown in FIGS. 1A-1B, article 10 comprises polymeric component 12 and a plurality of electrodes 14 disposed within polymeric component 12. While FIG. 1 illustrates an embodiment in which the article comprises a plurality of electrodes, the disclosure is not so limited, and that in certain embodiments, any appropriate numbers of electrodes (e.g., at least 1, at least 2, etc.) may be present, as described below.

In some embodiments, the one or more electrodes comprise stimulating electrodes, sensing electrodes, or combination thereof. In some cases, one or more of the electrodes may comprise a sensing electrode configured to sense a parameter and/or event associated with the GI tract. In some cases, the one or more electrodes may be configured to sense a parameter associated with an organ adjacent the GI tract (e.g., heart, lungs, etc.). In some embodiments, the one or more of the electrodes may comprise a stimulating electrode capable of applying an electrical stimulus to a location within the GI tract. In some embodiments, each of the one or more electrodes may advantageously function both as a sensing and stimulating electrode.

In some embodiments, the article further comprises one or more microfluidic channels disposed within the polymeric component. As used herein, “microfluidic channels” generally refer to channels having an average cross-sectional dimension of less than 1 mm. Referring again to FIGS. 1A-1B, in addition to comprising one or more electrodes 14, article 10 further comprises microfluidic channel 16 disposed within polymeric component 12. In some embodiments, the microfluidic channel and the associated one or more electrodes may be contained within a polymeric component having a relatively high aspect ratio (e.g., at least 10:1). While FIGS. 1A-1C illustrate an embodiment in which the article comprises a single microfluidic channel disposed in the polymeric component, the disclosure is not so limited, and that in certain embodiments, the article may comprise a plurality of microfluidic channels disposed within the polymeric component. In some embodiments, the one or more microfluidic channels may be in fluidic communication with a pump configured to control the flow of a chemical and/or therapeutic disposed within the one or more microfluidic channels, as described in more detail below.

It should be noted also that while FIG. 1A-1C illustrates an embodiment in which the article comprises both electrode(s) and microfluidic channel(s), the disclosure is not so limited. For example, in one set of embodiments, the article may comprise the electrode(s) disposed within the polymeric component but lack the microfluidic channel(s). Alternatively, in another set of embodiments, the article may comprise the microfluidic channel(s) but lack the electrode(s).

In some embodiments, the polymeric component has a relatively high aspect ratio, i.e., ratio of a largest dimension of the polymeric component relative to its cross-sectional dimension. As shown, polymeric component 12 comprises a relatively high aspect ratio, where a largest dimension L (e.g., length) is substantially larger than a cross-sectional dimension W (e.g., diameter, width, etc.). In some embodiments, the polymeric component may have an aspect ratio L/W of at least 10:1, at least 15:1, at least 20:1, at least 30:1, at least 40:1, at least 50:1, at least 75:1, at least 100:1, at least 250:1, at least 500:1, at least 750:1, at least 1,000:1, at least 2,500:1, at least 5,000:1, at least 7,500:1, at least 10,000:1, at least 25,000:1, at least 50,000:1, or at least 75,000:1. In some embodiments, the polymer component may have an aspect ratio L/W of less than or equal to 100,000:1, less than or equal to 75,000:1, less than or equal to 50,000:1, less than or equal to 25,000:1, less than or equal to 10,000:1, less than or equal to 7,500:1, less than or equal to 5,000:1, less than or equal to 2,500:1, less than or equal to 1,000:1, less than or equal to 750:1, less than or equal to 500:1, less than or equal to 250:1, less than or equal to 100:1, less than or equal to 75:1, less than or equal to 50:1, less than or equal to 40:1, less than or equal to 30:1, less than or equal to 20:1, or less than or equal to 15:1. Combination of the above-referenced ranges are possible (e.g., at least 10:1 and less than or equal to 1,000:1, or at least 10:1 and less than or equal to 100,000:1). Other ranges are also possible.

In some embodiments, the one or more microfluidic channels and/or one or more electrodes may have a largest dimension aligned parallel to the largest dimension of the polymeric component. As shown in FIG. 1A, microfluidic channel 16 has a largest dimension M aligned parallel to the largest dimension L of the polymeric component 12. Similarly, the one or more electrodes 14 may have a largest dimension E aligned parallel to the largest dimension L of polymeric component 12. In some embodiments, the one or more electrodes and/or the microfluidic channels may have a largest dimension that extends along the entirety of the polymeric component. For example, at least one of the one or more electrodes and/or one or more microfluidic channels (as shown in FIG. 1) may have a largest dimension (e.g., E or M) comparable to the largest dimension L of the polymeric component.

The one or more electrodes and/or microfluidic channels may be disposed within the polymeric component in any of a variety of configurations and arrangements. As shown in FIGS. 1A-1B, the microfluidic channel 16 may be centrally located within the polymeric component 12, and plurality of electrodes 14 may be positioned adjacent (e.g., around, etc.) the central microfluidic channel 16. While FIG. 1A can be used to illustrate one possible configuration of the microfluidic channel and the electrode within the polymeric component, the disclosure is not so limited, and in certain embodiments, the one or more electrodes and/or one or more microfluidic channels may be positioned in any appropriate locations within the polymeric component.

In some embodiments, the one or more microfluidic channels may be configured to house one or more therapeutics and/or chemicals. In some such embodiments, the therapeutics and/or chemicals may comprise chemicals capable of affecting (e.g., activating and/or inhibiting) the motility of one or more tissues associated with the GI tract and/or an organ adjacent the GI tract. For example, the chemical may comprise a neurochemical capable of affecting (e.g., activating and/or inhibiting) one or more nerves and/or a chemical capable of affecting (e.g., activating and/or inhibiting) one or more muscles associated with the GI tract. The therapeutics and/or chemicals may be present in the one or more microfluidic channels in any appropriate form, e.g., such as a liquid, a slurry, an emulsion, a suspension, etc. Examples of therapeutics and/or chemicals are provided below.

In some embodiments, the one or more electrodes disposed with the polymeric component are exposed to an external surrounding at predetermined locations along the polymeric component, thereby forming a plurality of electrode contacts. Any appropriate number of electrode contacts may be present in the article. In some embodiments, the exposed locations (i.e., electrode contacts) on the one or more electrodes along the polymeric component may serve as sensing and/or stimulating electrode contacts with the external surrounding, i.e., contacts capable of sensing and/or stimulating the external surrounding (e.g., GI tract).

In some embodiments, the one or more electrodes are exposed along a largest dimension of the polymeric component. FIG. 1C shows a non-limiting representation of a side view schematic of article 10. As shown FIG. 1C, one or more of the plurality of electrodes 14 aligned parallel to the largest dimension L of the polymeric component 12 may be exposed to an external surrounding at predetermined locations along polymeric component 12, thereby forming the plurality of electrode contacts (14A-14D).

In some embodiments, the one or more electrodes may be exposed to the external surrounding at predetermined locations having a controlled spacing. That is, the plurality of electrode contacts (e.g., electrode contacts 14A-14D) may be spaced apart by a certain spacing S. Alternatively, it may be possible to have variable spacings between the electrode contacts, e.g., such as spacing S_{A} between electrode contact 14A and 14B, and spacing S_{B} between electrode contact 14B and 14C, etc. The plurality of electrode contacts may have dimensions (e.g., width, length, height) that are the same or different. While FIG. 1C illustrates an embodiment in which the plurality of electrode contacts (e.g., 14A-14D) have identical dimensions, the disclosure is not so limited, and that in some embodiments, the plurality of electrode contacts may have variable dimensions.

While FIG. 1C illustrates an embodiment in which each of the one or more electrodes (e.g., electrodes 14(i)-14(iv)) comprises a plurality of electrode contacts (e.g., 14A-14D) having identical properties (e.g., arrangements, dimensions, spacings, etc.), it should be noted that disclosure is not so limited, and that in certain embodiments, the one or more electrodes may comprise a plurality of electrode contacts that differ in at least one property. A non-limiting example of one such embodiments is illustrated in FIG. 1D. As shown, the one or more electrodes 14 comprises electrodes 14(i)-14(iv) comprising electrode contacts having variable properties, e.g., such as the spacing between electrode contacts for electrode 14(i) and electrode 14(ii), dimension of electrode contacts for electrode 14(i) and electrode 14(iii), position of electrode contacts along the polymeric component for electrode 14(i) and electrode (iv), etc. In some embodiments, the plurality of electrode contacts may advantageously have a set of properties (e.g., dimensions, spacings, arrangements, etc.) that allows for efficient electrochemical of the external surrounding (e.g., a tissue in the GI tract).

In one set of embodiments, each of the one or more electrodes is exposed to an external surrounding via at least one electrode contact. In some cases, the electrode contact from each of the electrodes may be located at different locations along the largest dimension of the polymeric component. FIG. 1E can be used to illustrate one such embodiment. As shown, electrode 14(i) is exposed to the external surrounding via electrode contact 14A at a first location, electrode 14(ii) is exposed to the external surrounding via electrode contact 14B at a second location, electrode 14(iii) is exposed to the external surrounding via electrode contact 14C at a third location, etc. In some cases, the adjacent electrode contacts may be spaced apart by a certain spacing S.

The one or more electrodes may be exposed to an external surrounding at any of a variety of controlled spacings along the polymeric component. That is, the electrode contacts (e.g., 14A-14D in FIGS. 1C and 1E) may have any of a variety of spacing S. In some embodiments, the spacing S between the predetermined locations of the one or more electrodes (i.e., electrode contacts) may be at least 50 micrometers, at least 100 micrometers, at least 250 micrometers, at least 500 micrometers, at least 750 micrometers, at least 0.1 cm, at least 0.25 cm, at least 0.4 cm, at least 0.5 cm, at least 0.6 cm, at least 0.7 cm, at least 0.8 cm, at least 0.9 cm, at least 1 cm, at least 1.2 cm, at least 1.4 cm, at least 1.6 cm, at least 1.8 cm, at least 2 cm, at least 5 cm, at least 0.1 m, at least 0.5 m, at least 1 m, at least 1.5 m, at least 2 m, or at least 2.5 m. In some embodiments, the spacing S between the predetermined locations of the one or more electrodes (i.e., electrode contacts) may be less than or equal to 3 m, less than or equal to 2.5 m, less than or equal to 2 m, less than or equal to 1.5 m, less than or equal to 1 m, less than or equal to 0.5 m, less than or equal to 0.1 m, less than or equal to 5 cm, less than or equal to 2 cm, less than or equal to 2 cm, less than or equal to 1.8 cm, less than or equal to 1.6 cm, less than or equal to 1.4 cm, less than or equal to 1.2 cm, less than or equal to 1 cm, less than or equal to 0.9 cm, less than or equal to 0.8 cm, less than or equal to 0.7 cm, less than or equal to 0.6 cm, less than or equal to 0.5 cm, less than or equal to 0.25 cm, less than or equal to 0.1 cm, less than or equal to 750 micrometers, less than or equal to 500 micrometers, less than or equal to 250 micrometers, less than or equal to 100 micrometers. Combination of the above-reference ranges are possible (e.g., at least 50 micrometers and less than or equal to 3 m, or at least 50 micrometers and less than or equal to 2 cm, or at least 0.5 cm and less than or equal to 2 cm). Other ranges are also possible.

In an exemplary embodiment for illustrative purposes, FIG. 1F shows an exemplary article according to various aspects described herein, comprising a plurality of electrodes having a particular spacing (e.g., 1 cm). Other spacings and configurations are also possible and one of ordinary skill in the art would be capable of selecting such spacings and configurations based upon the teachings of this specification.

In some embodiments, the one or more electrodes may be optionally coated by one or more polymers. Referring again to FIG. 1B, the one or more electrodes 14 may be coated by a surface coating 18. The surface coating may comprise any of a variety of polymers described below (e.g., such as those described for the polymeric component 12). In one set of embodiments, the one or more polymers comprise any of a variety of thermoplastic polymer (e.g., perfluoroalkoxy alkane, etc.).

In some embodiments, the article comprises a plurality of pressure and/or strain sensors disposed along the length of the polymeric component. In one set of embodiments, the one or more electrodes contacts described herein (e.g., 14A-14D in FIG. 1C) may function as the pressure and/or strain sensors. For example, the one or more electrodes contacts, via impedance measurements, may be configured to sense a pressure and/or strain (e.g., a physiological pressure and/or strain) applied to the article (e.g., the pressure or strain exerted by a bolus of food). Alternatively or additionally, the one or more electrode contacts may be configured to sense electronystagmography (ENG), electromyography (EMG), and/or a local electrophysiological activity.

While FIG. 1C-4B illustrates an embodiment in which the one or more electrodes contacts described herein (e.g., 14A-14D in FIG. 1C) may function as the pressure and/or strain sensors, the disclosure is not so limited, and that in certain embodiments, other types pressure and/or strain sensors (non-electrode based sensors) may be disposed along the length of the polymeric component. In some such cases, the plurality of pressure and/or strain sensors may be configured to sense a pressure or strain (e.g., a physiological pressure or strain) applied to the article (e.g., the pressure exerted by a bolus of food). In some embodiments, the article may comprise a plurality of electrode contacts (e.g., 14A-14D in FIG. 1C). The article described herein (e.g., article 10 in FIGS. 1A-1C) may be formed by any of a variety of methods. Non-limiting examples of such methods include, but are not limited to lithographic techniques such as e-beam lithography, photolithography, X-ray lithography, extreme ultraviolet lithography, ion projection lithography, etc. Alternatively or additionally, a thermal drawing process may be employed to form the article described herein. In some embodiments, the thermal drawing process may comprise forming a macroscopic preform containing the one or more electrodes and optionally the one or more microfluidic channels, and subsequently stretching the preform to form the article described herein. In some such embodiments, an etching process may be employed to form the exposed electrode contacts along the length of the polymeric components.

In some embodiments, the article may be a part of a device and/or a system for treating GI motility disorders and/or performing neuromodulation in the GI tract. For example, in some such embodiments, the article is configured for implantation (e.g., submucosal or intramuscular implantation) in a location or an organ (e.g., in GI tract) internal a subject. In some embodiments, the location or organ internal the subject is the colon, the duodenum, the ileum, the jejunum, the stomach, the small intestine, the large intestine, the rectum, the mouth, or the esophagus. In an exemplary set of embodiments, the article is configured for implantation submucosally or intramuscularly in the esophagus and/or stomach to treat esophageal or gastric disorder.

According to some embodiments, upon submucosal or intramuscular implantation of the article in the location (e.g., GI tract) internal the subject, the article is configured to affect (e.g., activate or inhibit) one or more tissues (e.g., muscles and/or nerves) in the GI tract either electrically and/or chemically, e.g., via the one or more electrodes and/or the chemicals contained within the one or more microfluidic channels described above. Accordingly, via affecting the one or more tissues (e.g., muscles and/or nerves) associated with the location, the article may be used to treat GI motility disorders and/or performing neuromodulation in the GI tract, as described in more detail below.

In some embodiments, a system configured for treating GI motility disorders is disclosed herein. In some such embodiments, the system comprises an article configured for submucosal or intramuscular implantation in a location (e.g., GI tract) internal a subject. In some cases, the system may comprise an article described herein, e.g., as shown in FIGS. 1A-1E, which comprises a polymeric component and one or more electrodes.

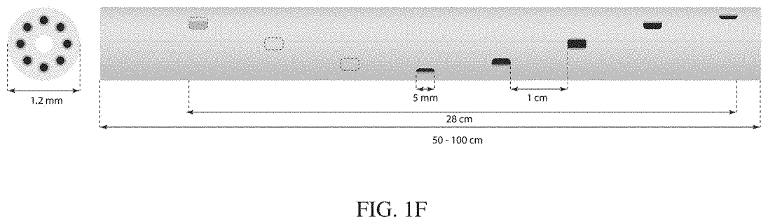

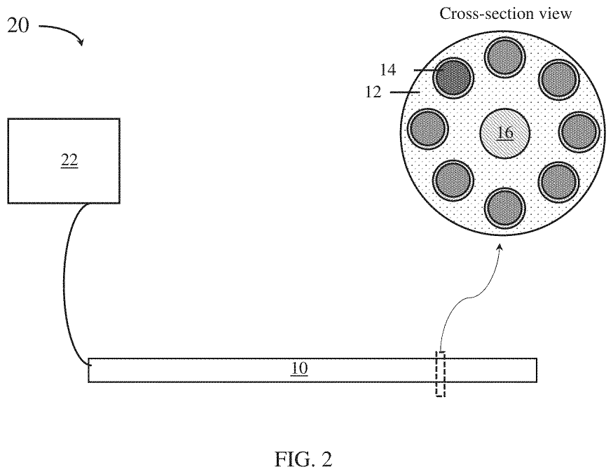

In some embodiments, the system further comprises a controller associated (e.g., in electrical communication) with the article. FIG. 2 shows a non-limiting representation of one such embodiment. As shown, system 20 comprises controller 22 associated (e.g., in electrical communication) with article 10, which comprises polymeric component 12 and the one or more electrodes 14. In some embodiments, the system described herein may further comprise one or more microchannels associated with the article configured for delivery of a therapeutic agent to a location (e.g., GI tract) internal to the subject. Referring to FIG. 2, article 10 associated with (e.g., electrically coupled to) the controller may comprise microfluidic channel 16, as illustrated by the perspective and cross-sectional schematics of article 10 in FIGS. 1A-1E. It should be understood that article 10 in system 20 may have any arrangement, configuration, properties, and components described elsewhere herein with respective to FIGS. 1A-1E.

In some embodiments, the controller is configured control the article, e.g., such as to stimulate the one or more electrodes and/or control release or delivery of a chemical and/or therapeutic contained within the microfluidic channel(s), upon sensing a bolus of food and/or detecting an event in the GI tract (e.g., esophagus, stomach, small intestine, and/or large intestine). In some cases, the event in the GI tract may be a contractile event (e.g., peristaltic contraction) and/or an inhibitory event (e.g., deglutitive inhibition). In some embodiments, upon sensing the bolus of food and/or detecting an event in the gastrointestinal tract, the controller is configured to stimulate the one or more electrodes via applying a voltage or current to the one or more electrodes.

In some embodiments, the controller described herein (e.g., controller 22 in FIG. 2) may be electrically connected to the one or more electrodes via either a wireless or a wired connection(s). For example, in one set of embodiments, the article or system described herein comprises wireless capabilities for allowing suitable communication with the controller (e.g., for controlling aspects of the article, the one or more electrodes and/or microfluidic channels, controlling/monitoring physiological conditions of the subject (e.g., at the location internal to the subject), etc.) and other devices and/or systems. Referring again to FIG. 2, article 10 may be associated with controller 22 wirelessly via a wireless device or transmitter. Wireless devices are generally known in the art and may include, in some cases, LTE, WiFi and/or Bluetooth systems. In some embodiments, the system described herein comprise such a wireless device. In some case, the wireless device or transmitter may be either implanted subcutaneously or may be placed external the subject. Alternatively, the controller may be electrically connected to the one or more electrodes and/or microfluidic channels in the article via wired connection(s).

In some embodiments, the controller described herein (e.g., controller 22 in FIG. 2) may be associated with the one or more microfluidic channels, e.g., such that the controller is capable of modulating a property of the chemical(s) or therapeutic(s) contained within the one or more microfluidic channels. In some cases, the property may be a temperature, a pressure, a flowrate, etc. In one set of embodiments, the controller may be electrically connected to a pump that is in fluidic communication (e.g., via a tube, etc.) with the one or more microfluidic channels. In some such embodiments, the pump, upon receiving a signal from the controller, may be capable of affecting the flow of chemical(s) or therapeutic(s) within the one or more microfluidic channels. For example, the pump may be configured to induce release or flow of chemical(s) or therapeutic(s) from the one or more channels. In some cases, the pump is a subdermal or epidermal infusion pump. In some cases, the pump may be in electrical communication with the controlled either via either a wireless or a wired connection.

In some embodiments, the controller described herein may be a closed-loop controller, i.e., a controller configured to stimulate (via a feedback loop) the article upon receiving information detected by the article in a coordinated fashion. For example, in some embodiments, the controller may be capable of controlling (e.g., actuating, stimulating) the article via a close-loop, either chemically and/or electrically, via the one or more electrodes and/or microchannels disposed within the polymeric component. The controller may be configured to control the article implanted at any of a variety of locations in the GI tract described above, e.g., such as the esophagus, stomach, small intestine, and/or large intestine.

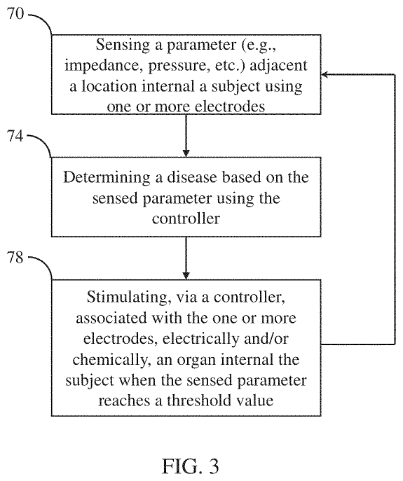

In some embodiments, a method (e.g., a close-loop method) for treating GI motility disorder using the article and/or system described above is disclosed herein. The GI motility disorders described herein may include hypomotility, spasticity, fibrotic, aganglionic, or hypermotility disorders associated with any of a variety of GI organs described herein, e.g., such as gastric and/or esophageal motility disorders. FIG. 3 shows a flow chart illustrating the plurality of steps associated with such a method of treating a GI motility disorder.

In some embodiments, the method comprises a step of sensing a parameter, a property, and/or a condition adjacent a location (e.g., GI tract) internal a subject using one or more electrodes and/or microfluidic channels associated with article described herein. Such a step of sensing using the one or more electrodes is illustrated by step 70 of FIG. 3. As noted above and described in FIG. 1C, the one or more electrodes may comprise exposed locations (i.e., electrode contacts 14A-14D) that allow for contact with an external surrounding (e.g., a location internal a subject) and sensing of a parameter adjacent the location.

In some embodiments, the sensed parameter, property, and/or condition comprises one or more of a measured impedance, pressure, electronystagmography (ENG), electromyography (EMG), and/or a local electrophysiological activity. In some cases, the sensed parameter or property may be associated with (e.g., indicative of) the presence of a bolus of food and/or a peristaltic event induced by the bolus of food in the GI tract. Examples of such a peristaltic event may include a contractile event (e.g., peristaltic contraction) and/or an inhibitory event (e.g., deglutitive inhibition).

In some embodiments, upon sensing a parameter, the one or more electrodes may communicate with a controller (e.g., a close-loop controller) associated with the one or more electrodes, such that the sensed information may be passed on to the controller for analysis. For example, as shown in FIG. 2, upon sensing a parameter (via the electrode contacts 14A-14D), the one or more electrodes 14 may communicate with the controller 22. In one set of embodiments, the controller may diagnose or determine a disease (e.g., specific GI motility disorder) based on the sensed parameter (e.g., as shown in step 74 of FIG. 3). Depending on the type of disease, the controller may be configured to transmit a specific set of commands to the article to trigger electrochemical stimulation. Alternatively, in one set of embodiments, upon sensing a parameter via the one or more electrodes, the controller may transmit a set of preprogrammed commands to the article to trigger electrochemical stimulation to target a known disease (e.g., a known GI motility disorder) in a subject. The system (e.g., system 20 in FIG. 2) described herein may be advantageously employed for both diagnostic and therapeutic purposes.

In some embodiments, the controller is configured to stimulate an organ internal the subject when the sensed parameter reaches a threshold value. Depending on the type of sensed parameter (e.g., impedance, pressure, ENG, EMG, etc.), the sensed parameter may be compared to a corresponding threshold value, e.g., a threshold value characteristic of a type of disease. As noted above, in accordance with certain embodiments, the article may further comprise a plurality of non-electrode based pressure or strain sensors disposed along the polymeric component. In some such embodiments, the controller may be configured to stimulate an organ internal the subject when the sensed pressure or strain detected by the plurality of pressure or strain sensors reaches a threshold value.

In some embodiments, when the sensed parameter reaches a threshold value, the controller is configured to stimulate an organ internal the subject electrically and/or chemically. In some embodiments, the step of stimulating an organ comprises stimulating the motility of an organ of the subject using the one or more (e.g., a plurality) of electrodes and/or a therapeutic and/or chemical contained within the one or more microfluidic channels.

For example, in one set of embodiments, the step of stimulating electrically an organ internal the subject comprises stimulating the organ via the one or more electrodes within an article. For example, as shown in FIG. 2, controller 22 may be configured to apply a voltage or current to the one or more electrode 14 in article 10, such that the one or more electrodes may stimulate the organ internal the subject via the exposed locations (e.g., electrode contacts 14A-14D in FIGS. 1C-1E) positioned along the largest dimension L of polymeric component 12.

In some embodiments, the step of stimulating chemically an organ internal the subject comprises exposing the organ internal the subject to a therapeutic and/or a chemical contained within a microfluidic channel. For example, referring to FIG. 2, system 20 may comprise microfluidic 16 associated with the one or more electrodes 14 and disposed within the polymeric component 12. The microfluid channel 16 may be configured to house a therapeutic and/or chemical. Upon actuation by the controller 22, the therapeutic and/or chemical may flow out of the microfluidic channel 16 to the external surrounding (e.g., an organ internal a subject).

In some embodiments, the controller operates in closed-loop with the one or more electrodes. For example, in one set of embodiments, after stimulating an organ internal the subject electrically and/or chemically via the controller, the one or more electrodes may be configured (for a second time) to sense a parameter adjacent a location internal a subject. FIG. 3 can be used to illustrate one such embodiment. As shown in FIG. 3, after stimulating electrically an/or chemically an organ internal the subject (step 78), a second round of sensing (step 70), determining (step 74), and/or stimulating (step 78) may occur. This closed-loop process may be repeated for any of a variety of appropriate duration and/or number of rounds (e.g., a third round, a fourth round, etc.).

In an exemplary set of embodiments, a method (e.g., a closed-loop method) for treating GI motility disorders is described herein. In some such embodiments, the method comprises steps of electrical sensing and stimulating the motility of an organ via electrical and/or chemical means using the article described herein.

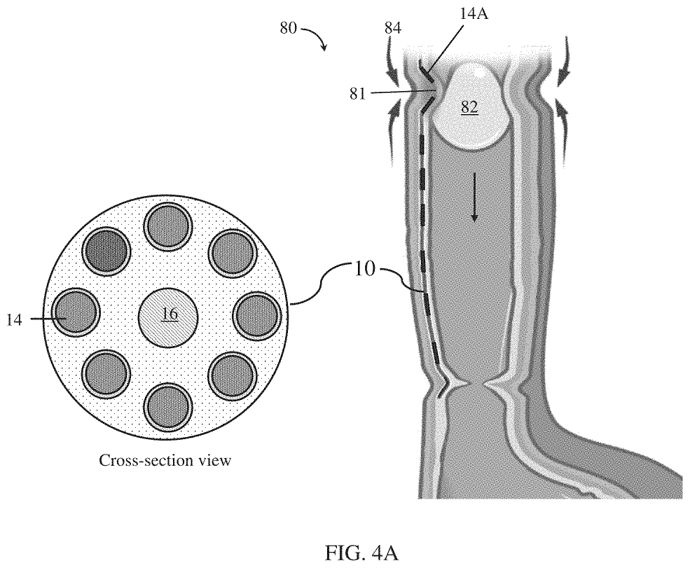

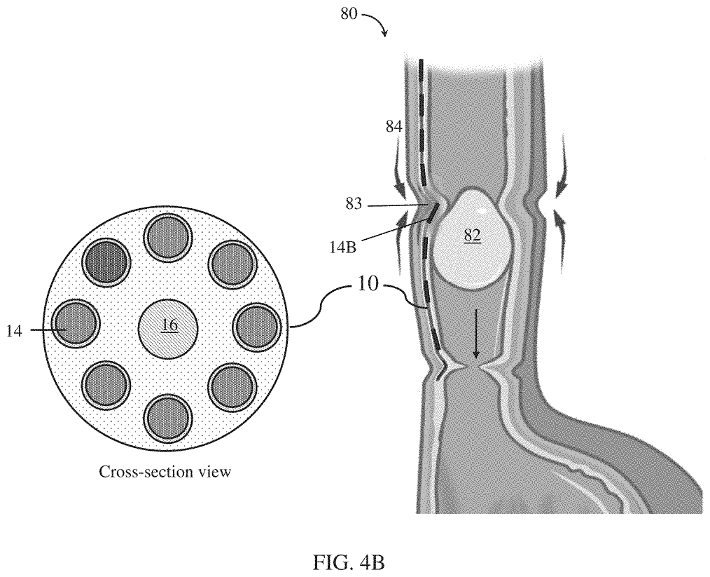

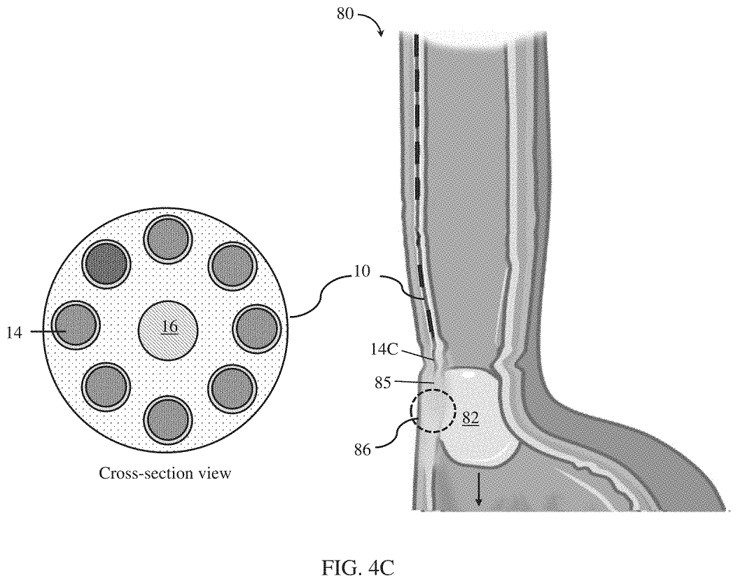

Certain embodiments comprise sensing, using a plurality of electrodes, a bolus of food present adjacent a location (e.g., GI tract) internal to a subject. The plurality of electrodes may be associated with an article described above (e.g., article 10 illustrated in FIGS. 1-2). A non-limiting representation of such a sensing step 80 is illustrated in FIG. 4A. As shown, an article 10 has been implanted into a submucosal or intramuscular layer of the GI tract, e.g., such as along a curvature of the esophagus. As shown, article 10 may have any of the features described in FIGS. 1-2, such as electrodes 14 comprising predetermined locations (e.g., electrode contact 14A, etc.) exposed to the submucosal or intramuscular layer. In some embodiments, upon submucosal or intramuscular implantation of article 10 into the GI tract, a bolus of food present adjacent a first location 81 may be sensed by the plurality of electrodes 14 via the electrode contact 14A adjacent the bolus of food along the GI tract.

In some embodiments, the step of sensing the bolus of food comprises sensing, via the plurality of electrodes, a pressure exerted by the bolus of food on the location internal the subject, an impedance change at the location internal the subject, contractile and/or inhibitory events (i.e., swallowing motion) induced by the bolus of food, and/or changes in ENG and EMG measurements.

In some embodiments, upon sensing of the bolus of food present adjacent the location (e.g., GI tract) internal to the subject, the plurality of electrodes may communicate with a controller (e.g., a close-loop controller as shown in FIG. 2) associated with the electrodes. The controller may, via application of a voltage or current, trigger the electrodes to stimulate motility of an organ in the subject. For instance, referring to FIG. 4A, the plurality of electrodes 14, upon sensing the bolus of food 82 via electrode contact 14A, may communicate with a controller (e.g., controller 22 as shown in FIG. 2) and receive signal to stimulate motility of an organ. In some embodiments, the controller may apply a voltage having an average magnitude of between 0.001 to 0.01 V, between 0.01 to 0.1 V, between 0.1 V and 10.0 V, between 1.0 V and 8.0 V, between 2.0 V and 5.0 V, between 0.1 V and 5.0 V, between 0.1 V and 1.5 V, between 0.1 V and 1.0 V, between 1.0 V and 3.0 V, between 3.0 V and 8.0 V, or any other appropriate range. In some embodiments, the controller may apply a current having an average magnitude of between 0.1 mA to 0.5 mA, between 0.5 mA to 1 mA, between 1 mA to 5 mA, between 5 mA to 10 mA, between 10 mA to 20 mA, between 20 mA to 30 mA, between 30 mA to 40 mA, between 40 mA to 50 mA, or any other appropriate range (e.g., between 0.1 mA to 50 mA).

The plurality of electrodes may stimulate motility of any of a variety of organs described herein, e.g., such as one or more organs in the GI tract (e.g., esophagus, stomach, small intestine, and/or large intestine). Referring to FIG. 4A, upon sensing the bolus of food 82 at the first location 81, the plurality of electrode 14 may stimulate motility of an organ (e.g., esophagus) of the subject.

In some embodiments, stimulating motility of an organ comprises stimulating the organ via neural activation and/or inhibition, muscular activation and/or inhibition, or combination thereof. For example, the one or more electrodes, via a controller, may be configured to apply programmed patterns of electric stimulation to the organ, thereby inducing contraction and/or inhibition the neuromusculature to enhance motility (e.g., peristalsis). Referring to FIG. 4A, the one or more electrodes may apply programmed patterns of electric stimulation 84 to first location 81 to stimulate motility of an organ (e.g., esophagus). Upon stimulating motility of the organ, the bolus of food 82 moves from the first location 81 to a second location 83 (e.g., as shown in FIG. 4B) in the GI tract.

In some embodiments, stimulating motility of the organ comprises creating propagated peristaltic waves that allow the bolus of food to move continuously along the GI tract from a first location to a second location, from a second location to a third location, etc. Such peristaltic waves may be made possible by the presence of a plurality of electrode contacts (e.g., 14A, 14B, etc.) that allows for continuously sensing and stimulating in a closed-loop operation. In some such embodiments, multiple rounds of sensing (the bolus of food) and stimulating of the organ may occur at various locations along the GI tract.

For example, as illustrated in FIGS. 4A-4B, certain embodiments further comprise sensing, using the plurality of electrodes, the bolus of food present adjacent a second location internal to a subject. As shown in FIG. 4B, the bolus of food present adjacent second location 83 may be sensed by plurality of electrodes 14 via electrode contact 14B at second location 83. In some embodiments, upon sensing of the bolus of food present adjacent the second location, the plurality of electrodes may again communicate with a controller (e.g., controller 22 as shown in FIG. 2) associated with the electrodes. Accordingly, the controller may again stimulate the electrodes via application of a voltage or current and instruct the electrodes to stimulate motility of the organ. As shown in FIG. 4B, upon sensing the bolus of food via electrode contact 14B at second location 83, the plurality of electrodes may stimulate motility of an organ of the subject via application of electric stimulation 84, thereby facilitating movement of the bolus of food 82 further along the GI tract. The process may be repeated a number of times, until the bolus of food reaches a desired location. For example, as illustrated in FIG. 4C, as bolus of food 82 reaches third location 85, the bolus of food may be sensed by electrode contact 14C, which then may stimulate the motility of an organ (e.g., esophagus and/or stomach) to facilitate movement of the bolus of food further along the GI tract.

While FIGS. 4A-4B illustrate an embodiment in which the method of treating GI motility disorders includes electrical stimulation of organ motility in the GI tract, the disclosure is not so limited, and that in certain embodiments, chemical stimulation may also be employed.

In some embodiments, the method for treating GI motility disorders further comprises flowing, in a microfluidic channel associated with the plurality of electrodes, a therapeutic agent and/or chemical. FIG. 4C can be used to illustrate such an embodiment. As shown, upon sensing the bolus of food at third location 85, article 10 may be configured to flow, in a microfluidic channel 16, a therapeutic agent and/or chemical to a location adjacent the bolus of food 82. In some such embodiments, a chemical stimulation is employed to stimulate the motility of the organ in the GI tract via the release of the therapeutic agent and/or chemical. Accordingly, such stimulated organ motility may translate into contraction and/or inhibition of the neuromusculature (i.e., peristalsis) and facilitate the movement of the bolus of food along the GI tract. For example, as shown in FIG. 4C, via release of therapeutic agent and/or chemical contained within microfluidic channel 16, chemical stimulation 86 may be applied to stimulate the motility of the organ in the GI tract (e.g., esophagus), which in turn facilitates the movement of bolus of food 82 in the GI tract.

In some embodiments, article and/or systems and interrelated methods for neuromodulation are disclosed herein. In some such embodiments, the devices and/or systems may be configured to stimulate the neuromusculature of one or more organs in the GI tract of subject to mimic hormonal responses of a fed state (e.g., a state after ingestion of a bolus of food) in a subject, as described in more detail below. Such devices and/or systems may be particularly beneficial in modulating satiety in a subject when the subject is in a fasted state.

In some embodiments, a system for neuromodulation comprises an article configured to stimulate one or more tissues associated with the GI tract (e.g., esophagus, stomach, small intestine, and/or large intestine) in a subject using a plurality of electrodes. In some embodiments, the article may comprise a polymeric component, the one or more electrodes disposed within the polymeric component, and a microfluidic channel disposed within the polymeric component. A non-limiting example of such an article is shown FIGS. 1A-1E (e.g., article 10). The article and associated components may have any properties, dimensions, and/or arrangements previously described with respect to FIGS. 1A-1E. For instance, the one or more electrodes may comprise at least two electrodes, e.g., a sensing electrode and a stimulating electrode.

In some embodiments, the article, via stimulation of the one or more electrodes, may be capable of inducing production of metabolic hormones at a level that mimics a level of hormones produced in fed state in a subject. In some such embodiments, a fed state may be used to refer a state after peristalsis followed by ingestion of a bolus of food in a subject. Examples of metabolic hormones typically produced when the subject is in a fed include, but are not limited to, metabolic hormones are selected from the group of GLP-1, insulin, glucagon, GIP, glucose, and ghrelin.

In some embodiments, the system for neuromodulations comprises a controller (e.g., a closed-loop controller). In some embodiments, the controller may be configured to actuate the one or more electrodes to stimulate the one or more tissue to induce production of metabolic hormones. A non-limiting example of such a system for neuromodulation is shown in FIG. 2. As shown, system 20 comprises the controller 22 in electrical communication with the article 10. The system and controller may have any properties, dimensions, and/or configurations described previously with respect to FIG. 2.

In some embodiments, a method of neuromodulation using an article (e.g., article 10 in FIGS. 1A-1E) and/or a system (e.g., system 20 in FIG. 2) is disclosed herein.

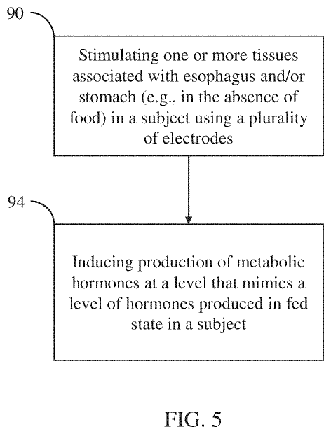

Certain embodiments comprise stimulating one or more tissues associated with one or more organs in the GI tract (e.g., esophagus, stomach, small intestine, and/or large intestine) in a subject using a plurality of electrodes, when the subject is in a fasted state. That is, the step of stimulating the one or more tissues occurs in the absence of a bolus of food adjacent a location in the GI tract (e.g., esophagus, stomach, small intestine, and/or large intestine) of the subject. FIG. 5 can be used to illustrate such a step of stimulating (e.g., Step 90). In some embodiments, the one or more tissues comprise one or more muscles and/or nerves associated with the gastrointestinal tract. In some embodiments, the one or more nerves include vagal efferent nerves.

In some embodiments, upon stimulation, the one or more stimulated tissues (e.g., nerve tissues) in the GI tract may be configured to communicate with nervous systems in the brain. In some such embodiments, the nervous systems in the brain may in turn send signals to the GI tract, thereby inducing the production of metabolic hormones in the GI tract. Examples of nervous systems include, but are not limited to, nucleus tractus solitarius (NTS), dorsal motor nucleus (DMN), and hypothalamus.

In some embodiments, upon stimulating the one or more tissues associated with the one or more organs in the GI tract, a production of metabolic hormones may be induced at a level that mimics a level of hormones produced in fed state in a subject. FIG. 5 can be used to illustrate such a step of inducing hormone production (e.g., Step 94). The types and levels of hormones produced may differ depending on the type of stimulated tissues and organs. For example, the type and level of hormones induced via stimulation of one or more tissues associated with the esophagus may differ from those induced via stimulation of one or more tissues associated with the stomach.

In some embodiments, the stimulation may be applied by the plurality of electrodes to the one or more tissues in any appropriate manner. For example, in one set of embodiments, the one or more tissues may be stimulated continuously for a certain duration. In another set of embodiments, the one or more tissues may be stimulated periodically (e.g., with periods of stimulation following by periods of rest) for a certain total duration.

In some embodiments, the one or more tissues may be stimulated for a total duration of at least 1 second, at least 5 seconds, at least 10 seconds, least 25 seconds, least 50 seconds, least 100 seconds, least 120 seconds, least 300 seconds, least 480 seconds, at least 600 seconds, at least 960 seconds, at least 1,200 seconds, at least 1,800 seconds, at least 2,700 seconds, at least 5,400 seconds, at least 7,200 seconds, at least 9,000 seconds, at least 12,000 seconds, at least 18,000 seconds, at least 36,000 seconds, at least 48,000 seconds, at least 60,000 seconds, or at least 72,000 seconds. In some embodiments, the one or more tissues may be stimulated for a total duration of less than or equal to 86,400 seconds, less than or equal to 72,000 seconds, less than or equal to 60,000 seconds, less than or equal to 48,000 seconds, less than or equal to 36,000 seconds, less than or equal to 18,000 seconds, less than or equal to 12,000 seconds, less than or equal to 9,000 seconds, less than or equal to 7,200 seconds, less than or equal to 5,400 seconds, less than or equal to 2,700 seconds, less than or equal to 1,800 seconds, less than or equal to 1,200 seconds, less than or equal to 960 seconds, less than or equal to 600 seconds, less than or equal to 480 seconds, less than or equal to 300 seconds, less than or equal to 120 seconds, less than or equal to 100 seconds, less than or equal to 50 seconds, less than or equal to 25 seconds, less than or equal to 10 seconds, or less than or equal to 5 seconds. Any of the above referenced values may be possible (e.g., at least 1 second and less than or equal to 86,400 seconds, at least 120 seconds and less than or equal to 9,000 seconds, etc.). Other ranges are also possible.

In embodiments in which the one or more tissues are stimulated periodically, the one or more tissues may be stimulated using any of a variety of appropriate periods of stimulation. In some embodiments, the one or more tissues may be stimulated for a period of at least 1 second, at least 5 seconds, at least 10 seconds, 20 seconds, at least 40 seconds, at least 60 seconds, at least 80 seconds, at least 100 seconds, at least 120 seconds, at least 140 seconds, at least 160 seconds, at least 200 seconds, at least 400 seconds, at least 800 seconds, at least 1,200 seconds, or at least 1,600 seconds. In some embodiments, the one or more tissues may be stimulated for a period of less than or equal to 1,800 seconds, less than or equal to 1,600 seconds, less than or equal to 1,200 seconds, less than or equal to 800 seconds, less than or equal to 400 seconds, less than or equal to 200 seconds, less than or equal to 180 seconds, less than or equal to 160 seconds, less than or equal to 140 seconds, no more 120 seconds, less than or equal to 100 seconds, less than or equal to 80 seconds, less than or equal to 60 seconds, less than or equal to 40 seconds, less than or equal to 20 seconds, less than or equal to 10 seconds, or less than or equal to 5 seconds. Any of the above referenced values may be possible (e.g., at least 1 second and less than or equal to 1800 seconds, at least 20 seconds and less than or equal to 200 seconds). Other ranges may be possible.

Any appropriate rest periods may be employed between the periods of stimulation. In some embodiments, a rest period of at least 1 second, at least 5 seconds, 10 seconds, at least 20 seconds, at least 30 seconds, at least 40 seconds, at least 50 seconds, at least 60 seconds, at least 80 seconds, at least 120 seconds, at least 200 seconds, at least 400 seconds, at least 800 seconds, at least 1,200 seconds, or at least 1,600 seconds may be employed. In some embodiments, a rest period of less than or equal to 1,800 seconds, less than or equal to 1,600 seconds, less than or equal to 1,200 seconds, less than or equal to 800 seconds, less than or equal to 400 seconds, less than or equal to 200 seconds, less than or equal to 120 seconds, less than or equal to 80 seconds, less than or equal to 60 seconds, less than or equal to 50 seconds, less than or equal to 40 seconds, less than or equal to 30 seconds, less than or equal to 20 seconds, less than or equal to 10 seconds, or less than or equal to 5 seconds may be employed. Any of the above referenced values may be possible (e.g., at least 1 second and less than or equal to 1,800 seconds, at least 10 seconds and less than or equal to 60 seconds). Other ranges are also possible.

In some embodiments, the article (e.g., article 10 in FIGS. 1-2) may be configured to adjust various parameters based on physiological and/or external metrics. In some embodiments, the article is associated with one or more microfluidic channels configured for the release of a pharmaceutical agent. For example, in some embodiments, the article is configured to adjust the rate and/or amount of a pharmaceutical agent released from the article (e.g., stored within one or more microfluidic channels associated with the residence article) e.g., in response to a signal from a controller in electrical or wireless communication with and/or associated with (e.g., embedded within) the article. In some embodiments, the article adjusts the rate and/or amount of a pharmaceutical agent released from the article in response to an input from the user and/or a signal from the controller.

In some cases, the electrodes may include a conductive material, e.g., metals, metal alloys, metal oxides, and/or semiconductors, such as those described herein. Such metals may be exposed to the external environment (for example, the article once introduced into a subject), and accordingly, in some cases, such electrodes may be used to determine a physical property of a subject, and/or provide a stimulus (e.g., an electrical stimulus) to a subject. The electrode may include materials selected from metals such as stainless steel, aluminum, gold, silver, copper, molybdenum, tantalum, titanium, nickel, tungsten, chromium, palladium, platinum, as well as any combinations of these and/or other metals, semiconductor materials such as silicon, gallium, germanium, diamond (carbon), tin, indium, graphene (carbon), carbon nanotube fiber selenium, tellurium, boron, phosphorous, and/or other semiconductors described herein, metal alloys or oxides such as platinum iridium, iridium oxide, and/or various other conductive materials such as conductive fibers, carbon fiber, carbon nanotube fiber, and/or various other organic electronics such as conductive polymers.

Any of an appropriate number of electrodes (e.g., one or more electrodes 14 in FIGS. 1A-1E) may be disposed within the polymeric component in an article. In some embodiments, the article comprises at least 1, at least 2, at least 3, at least 4, at least 5, at least 6, at least 7, at least 8, at least 9, at least 10, at least 12, at least 15, at least 20, at least 40, at least 60, or at least 80 electrodes disposed within the polymeric component. In some embodiments, the article comprises less than or equal to 100, no more least 80, less than or equal to 60, less than or equal to 40, less than or equal to 20, less than or equal to 15, less than or equal to 12, less than or equal to 10, less than or equal to 9, less than or equal to 8, less than or equal to 7, less than or equal to 6, less than or equal to 5, less than or equal to 4, less than or equal to 3, or less than or equal to 2 electrodes disposed within the polymeric component. Any of the above-reference range are possible (e.g., at least 1 and less than or equal to 100). Other ranges are possible.

In one set of embodiments, the article may include a plurality of electrodes (e.g., at least two electrodes) disposed within the polymeric component. Advantageously, the presence of a plurality of electrodes in the article, compared to a single electrode contact on the device, may impart the article with certain capabilities. For example, the plurality of electrodes may include at least a stimulating electrode and at least a sensing electrode, and thus impart the article with simultaneous sensing and stimulating capabilities. In some cases, each of the one or more electrodes may advantageously act as both the sensing and stimulating electrode. In some cases, the plurality of electrodes may advantageously allow for the propagation of neuromuscular waves in dysmotile organs.

Any of an appropriate number of electrode contacts (e.g., electrode contacts 14A-14D) may be disposed along the polymeric component in an article. In some embodiments, for each of the one or more electrodes (e.g., electrode 14(i)-14(iv) in the one or more electrodes 14 in FIG. 1C-1E), at least 1, at least 2, at least 3, at least 4, at least 5, at least 6, at least 7, at least 8, at least 9, at least 10, at least 12, at least 15, at least 20, at least 40, at least 60, or at least 80 electrodes contacts may be disposed along the polymeric component. In some embodiments, for each of the one or more electrodes (e.g., electrode 14(i)-14(iv) in the one or more electrodes 14 in FIG. 1C-1E), less than or equal to 100, no more least 80, less than or equal to 60, less than or equal to 40, less than or equal to 20, less than or equal to 15, less than or equal to 12, less than or equal to 10, less than or equal to 9, less than or equal to 8, less than or equal to 7, less than or equal to 6, less than or equal to 5, less than or equal to 4, less than or equal to 3, or less than or equal to 2 electrode contacts are disposed along the polymeric component. Any of the above-reference range are possible (e.g., at least 1 and less than or equal to 100). Other ranges are possible.

The one or more electrodes may comprise any of an appropriate number of total electrode contacts. In some embodiments, the one or more electrodes (e.g., the one or more electrodes 14 in FIG. 1C) may have a total of at least 1, at least 2, at least 3, at least 4, at least 5, at least 6, at least 7, at least 8, at least 9, at least 10, at least 12, at least 15, at least 20, at least 40, at least 60, at least 80, at least 100, at least 200, at least 500, at least 1,000, at least 2,500, at least 5,000, or at least 7,500 electrodes contacts disposed along the polymeric component. In some embodiments, the one or more electrodes (e.g., the one or more electrodes 14 in FIG. 1C) may have a total of less than or equal to 10,000, less than or equal to 7,500, less than or equal to 5,000, less than or equal to 2,500, less than or equal to 1,000, less than or equal to 500, less than or equal to 200, less than or equal to 100, less than or equal to 80, less than or equal to 60, less than or equal to 40, less than or equal to 20, less than or equal to 15, less than or equal to 12, less than or equal to 10, less than or equal to 9, less than or equal to 8, less than or equal to 7, less than or equal to 6, less than or equal to 5, less than or equal to 4, less than or equal to 3, or less than or equal to 2 electrode contacts are disposed along the polymeric component. Any of the above-reference range are possible (e.g., at least 1 and less than or equal to 10,000, at least 1 and less than or equal to 100). Other ranges are possible.

In some embodiments, the article described herein (e.g., article 10 in FIG. 1C) comprises at least two electrode contacts disposed along a largest dimension of the polymeric component (e.g., polymeric component 12 in FIG. 1C). In one set of embodiments, the at least two electrode contacts may be from a single electrode (e.g., electrode contacts 14A-14D on electrode 14(i) in FIG. 1C). Alternatively, the at least two electrode contacts may be from different electrodes (e.g., electrode contact 14A on electrode 14(i), electrode contact 14B on electrode 14(ii) in FIG. 1E). Advantageously, the presence of at least 2 total electrode contacts along the polymeric component may allow for the propagation of neuromuscular waves in dysmotile organs (e.g., GI organs) located internal the subject, as described in more detail below.

The one or more electrodes may have any of a variety of appropriate shapes. The electrodes may have any appropriate cross-sectional shape, for example, square, rectangular, circular, trapezoidal, or the like. For example, the one or more electrodes may be rod-like, e.g., such as microwire or nanowire.

In some embodiments, the one or more electrodes (e.g., one or more electrodes 14 in FIG. 1A) has a relatively high aspect ratio, i.e., a ratio of a largest dimension of the electrode that is aligned parallel to the largest dimension of the polymeric component versus a cross-sectional dimension of the electrode (e.g., diameter, width, etc.). In some embodiments, the one or more electrodes may have an aspect ratio of at least 100:1, at least 250:1, at least 500:1, at least 750:1, at least 1,000:1, at least 2,500:1, at least 5,000:1, at least 7,500:1, at least 10,000:1, at least 25,000:1, at least 50,000:1, at least 75,000:1, at least 100,000:1, at least 150,000:1, at least 200,000:1, or at least 250,000:1. In some embodiments, the one or more electrodes may have an aspect ratio of less than or equal to 300,000:1, less than or equal to 250,000:1, less than or equal to 200,000:1, less than or equal to 150,000:1, less than or equal to 100,000:1, less than or equal to 75,000:1, less than or equal to 50,000:1, less than or equal to 25,000:1, less than or equal to 10,000:1, less than or equal to 7,500:1, less than or equal to 5,000:1, less than or equal to 2,500:1, less than or equal to 1,000:1, less than or equal to 750:1, less than or equal to 500:1, or less than or equal to 250:1. Combination of the above-referenced ranges are possible (e.g., at least 100:1 and less than or equal to 300,000:1 less than or equal to). Other ranges are also possible.

The one or more electrodes may have any of a variety of sizes (e.g., a cross-sectional dimension, a largest dimension parallel to the largest dimension of the polymeric component). In some embodiments, the one or more electrodes may have a cross-sectional dimension (e.g., diameter) of at least 10 micrometers, at least 15 micrometers, at least 20 micrometers, at least 25 micrometers, at least 50 micrometers, at least 75 micrometers, at least 100 micrometers, at least 150 micrometers, at least 200 micrometers, at least 250 micrometers, at least 300 micrometers, at least 350 micrometers, at least 400 micrometers, at least 500 micrometers, at least 1 mm, at least 1.5 mm, at least 2 mm, at least 3 mm, at least 4 mm, at least 5 mm, at least 10 mm, at least 20 mm, at least 50 mm, or at least 100 mm. In some embodiments, the one or more electrodes may have a cross-sectional dimension (e.g., diameter) of less than or equal to 500 mm, less than or equal to 200 mm, less than or equal to 100 mm, less than or equal to 50 mm, less than or equal to 20 mm, less than or equal to 10 mm, less than or equal to 5 mm, less than or equal to 4 mm, less than or equal to 3 mm, less than or equal to 2 mm, less than or equal to 1.5 mm, less than or equal to 1 mm, less than or equal to 500 micrometers, less than or equal to 400 micrometers, less than or equal to 350 micrometers, less than or equal to 300 micrometers, less than or equal to 250 micrometers, less than or equal to 200 micrometers, less than or equal to 150 micrometers, less than or equal to 100 micrometers, less than or equal to 75 micrometers, less than or equal to 50 micrometers, less than or equal to 25 micrometers, less than or equal to 20 micrometers, or less than or equal to 15 micrometers. Any of the above-referenced ranges may be possible (e.g., at least 25 micrometers and less than or equal to 2 mm). Other ranges are also possible.

The one or more electrodes may have any of a variety of values of a largest dimension (e.g., E in FIG. 1A). In some embodiments, the one of more electrodes may have a largest dimension of at least 2 cm, at least 5 cm, at least 10 cm, at least 25 cm, at least 50 cm, at least 75 cm, at least 1 m, at least 1.25 m, at least 1.5 m, at least 1.75 m, at least 2 mm, or at least 2.5 m. In some embodiments, the one or more electrodes may have a largest dimension of less than or equal to 3 m, less than or equal to 2.5 m, less than or equal to 2.25 m, less than or equal to 2 m, less than or equal to 1.75 m, less than or equal to 1.5 m, less than or equal to 1.25 m, less than or equal to 1 m, less than or equal to 75 cm, less than or equal to 50 cm, less than or equal to 25 cm, less than or equal to 10 cm, or less than or equal to 5 cm. Any of the above-referenced ranges may be possible (e.g., at least 2 cm and less than or equal to 3 m). Other ranges are also possible.

It should be noted that in embodiments in which the article comprises more than one electrode, the electrodes may be the same or different with respect to one or more properties described above (e.g., material, type, shape, spacing, etc.).

The polymeric component (and/or the article) may have any of a variety of sizes (e.g., a cross-sectional dimension W, a largest dimension L as shown in FIG. 1A). In some embodiments, the polymeric component (and/or article) may have a cross-sectional dimension W (e.g., a diameter) of at least 0.1 mm, at least 0.25 mm, at least 0.5 mm, at least 0.75 mm, at least 1 mm, at least 1.25 mm, at least 1.5 mm, at least 1.75 mm, at least 2 mm, or at least 2.25 mm. In some embodiments, the polymeric component may have a cross-sectional dimension W (e.g., a diameter) of less than or equal to 3 mm, less than or equal to 2.5 mm, less than or equal to 2.25 mm, less than or equal to 2 mm, less than or equal to 1.75 mm, less than or equal to 1.5 mm, less than or equal to 1.25 mm, less than or equal to 1 mm, less than or equal to 0.75 mm, less than or equal to 0.5 mm, or less than or equal to 0.25 mm. Any of the above-referenced ranges may be possible (e.g., at least 0.1 mm and less than or equal to 2.5 mm). Other ranges are also possible.

The polymeric component (and/or article) may have any of a variety of values of a largest dimension (e.g., length such as L in FIG. 1A). In some embodiments, the polymeric component (and/or article) may have a largest dimension of at least 2 cm, at least 5 cm, at least 7.5 cm, at least 0.1 m, at least 0.25 m, at least 0.5 m, at least 0.75 m, at least 1 m, at least 1.25 m, at least 1.5 m, at least 1.75 m, at least 2 mm, or at least 2.5 mm. In some embodiments, the polymeric component (and/or article) may have a largest dimension of less than or equal to 3 m, less than or equal to 2.5 m, less than or equal to 2.25 m, less than or equal to 2 m, less than or equal to 1.75 m, less than or equal to 1.5 m, less than or equal to 1.25 m, less than or equal to 1 m, less than or equal to 0.75 m, less than or equal to 0.5 m, less than or equal to 0.25 m, less than or equal to 0.1 m, less than or equal to 7.5 cm, or less than or equal to 5 cm. Any of the above-referenced ranges may be possible (e.g., at least 2 cm and less than or equal to 3 m). Other ranges are also possible.

The polymeric component described herein (e.g., 12 in FIGS. 1A-1D) may have any of a variety of appropriate shapes, including, but not limited to, e.g., tubes, rods, cylinders, and cross-sections with square, polygonal, or round profiles.

The polymeric component described herein may comprise any of a variety of polymers or elastomers. In some embodiments, the polymeric component (e.g., polymeric component 12 in FIGS. 1-2) is elastic in nature. In some such embodiments, the elastic polymeric component is relatively flexible. In certain embodiments, the elastic polymeric component may be selected such that it is capable of undergoing large angle deformation for relatively long periods of time without undergoing significant non-elastic deformation. In some such embodiments, the elastic polymeric component may have a strength of recoil sufficient to substantially return the elastic polymeric component to its pre-deformed shape within less than about 30 minutes, within less than about 10 minutes, within less than about 5 minutes, or within less than about 1 minute after release of the mechanical deformation. Those skilled in the art would understand that returning to its pre-deformed shape shall be understood to not require absolute conformance to a mathematical definition of shape, but, rather, shall be understood to indicate conformance to the mathematical definition of shape to the extent possible for the subject matter so characterized as would be understood by one skilled in the art most closely related to such subject matter.

In some embodiments, the elastic polymeric component (e.g., polymeric component 12 in FIGS. 1-2) has a particular elastic modulus. In some embodiments, the elastic modulus of the elastic polymeric component ranges between about 0.1 MPa and about 100 MPa. In some embodiments, the elastic modulus of the elastic polymeric component is at least about 0.1 MPa, at least about 0.2 MPa, at least about 0.3 MPa, at least about 0.5 MPa, at least about 1 MPa, at least about 2 MPa, at least about 5 MPa, at least about 10 MPa, at least about 25 MPa, at least about 50 MPa, or at least about 75 MPa. In certain embodiments, the elastic modulus of the elastic polymeric component is less than or equal to 100 MPa, less than or equal to about 75 MPa, less than or equal to about 50 MPa, less than or equal to about 25 MPa, less than or equal to about 10 MPa, less than or equal to about 5 MPa, less than or equal to about 2 MPa, less than or equal to about 1 MPa, less than or equal to about 0.5 MPa, less than or equal to about 0.3 MPa, or less than or equal to about 0.2 MPa. Combinations of the above referenced ranges are also possible (e.g., between 0.1 MPa and about 100 MPa, between about 0.1 MPa and about 30 MPa, between about 0.3 MPa and about 10 MPa). Other ranges are also possible. Those skilled in the art would be capable of selecting suitable methods for determining the elastic modulus of a polymeric component including, for example, tensile mechanical characterization under ASTM D638 and/or compressive mechanical characterization under ASTM D575.

In some embodiments, the elastic polymeric component (e.g., polymeric component 12 in FIGS. 1-2) undergoes a relatively low amount of creep during mechanical deformation. For example, in certain embodiments, the elastic polymeric component has a minimum creep rate of less than or equal to about 0.3 mm/mm/hr, less than or equal to about 0.2 mm/mm/hr, less than or equal to about 0.1 mm/mm/hr, less than or equal to about 0.08 mm/mm/hr, less than or equal to about 0.05 mm/mm/hr, less than or equal to about 0.03 mm/mm/hr, or less than or equal to about 0.02 mm/mm/hr. In certain embodiments, the elastic polymeric component has a minimum creep rate of at least about 0.01 mm/mm/hr, at least about 0.02 mm/mm/hr, at least about 0.03 mm/mm/hr, at least about 0.05 mm/mm/hr, at least about 0.08 mm/mm/hr, at least about 0.1 mm/mm/hr, or at least about 0.2 mm/mm/hr. Combinations of the above referenced ranges are also possible (e.g., between about 0.01 mm/mm/hr and about 0.3 mm/mm/hr, between about 0.02 mm/mm/hr and about 0.1 mm/mm/hr, between about 0.02 mm/mm/hr and about 0.05 mm/mm/hr, between about 0.05 mm/mm/hr and about 0.3 mm/mm/hr). Other ranges are also possible. Minimum creep rate can be determined, in some embodiments, according to ASTM D-638. Briefly, a sheet of the elastic polymeric material is prepared, as described below, and cut into a standard dumbbell die. The specimens can be loaded into grips of an Instron testing machine and the gauge length measured using a digital micrometer. A constant stress corresponding to 30% of the ultimate tensile strength of each material may be applied to the specimens for 60 min at constant temperature (e.g., room temperature) and the creep (in mm/mm) versus time (in hours) can be plotted. The minimum creep rate is the slope of the creep vs. time curve prior to secondary creep.

Those skilled in the art would be capable of determining suitable methods for tuning the mechanical properties (e.g., elastic modulus, creep behavior) of the elastic polymeric component by, for example, varying the molar ratios of monomeric and/or polymeric units (e.g., increasing the amount of high molecular weight polymers used in the elastic polymeric component), varying polymer cross-linking density, varying the concentration of cross-linking agents used in the formation of the polymer, varying the crystallinity of the polymer (e.g., by varying the ratio of crystalline and amorphous regions in the polymer) and/or the use of additional or alternative materials (e.g., incorporating materials such as bis(isocyanatomethyl)-cyclohexane).

In some embodiments, the elastic polymeric component (e.g., polymeric component 12 in FIGS. 1-2) does not substantially swell in the presence of biological fluids such as blood, water, bile, gastric fluids, and/or the like. In some embodiments, the elastic polymer component swells between about 0.01 vol % and about 10 vol % in a biological fluid as compared to the volume of the elastic polymer component in the dry state (e.g., at atmospheric conditions and room temperature). For example, in certain embodiments, the elastic polymeric component swells by less than about 10 vol %, less than about 5 vol %, less than about 2 vol %, or less than about 1 vol % in a biological fluid as compared to the volume of the elastic polymeric component in the dry state (e.g., at atmospheric conditions and room temperature). Those skilled in the art would be capable of selecting suitable methods for determining the amount of swelling of an elastic polymeric component based upon the teachings of this specification including, for example, measuring the volume of the elastic polymeric component in the dry state at atmospheric conditions and room temperature, submerging the component in a biological fluid (e.g., blood, water, bile, gastric fluids, and/or the like) and measuring the percent change in volume of the component after about 60 minutes.

The polymeric component (e.g., polymeric component 12 in FIGS. 1-2) is generally biocompatible. The term “biocompatible,” as used herein, refers to a polymer that does not invoke an adverse reaction (e.g., immune response) from an organism (e.g., a mammal), a tissue culture or a collection of cells, or if the adverse reaction does not exceed an acceptable level.

In some embodiments, the elastic polymeric component comprises polymers, their networks, and/or multi-block combinations of, for example, polyesters, including but not limited to, polycaprolactone, poly(propylene fumarate), poly(glycerol sebacate), poly(lactide), poly(glycol acid), poly(lactic-glycolic acid), polybutyrate, and polyhydroxyalkanoate; polyethers, including but not limited to, poly(ethylene oxide) and poly(propylene oxide); polysiloxanes, including but not limited to, poly(dimethylsiloxane); polyamides, including but not limited to, poly(caprolactam); polyolefins, including but not limited to, polyethylene; polycarbonates, including but not limited to poly(propylene oxide); polyketals; polyvinyl alcohols; polyoxetanes; polyacrylates/methacrylates, including but not limited to, poly(methyl methacrylate) and poly(ethyl-vinyl acetate); polyanhydrides; polytetrafluoroethylene (PTFE); and polyurethanes (e.g., thermoplastic polyurethanes). In some embodiments, the polymer is cross-linked. In some embodiments, the elastic polymeric component comprises a polymer composite comprising two or more chemically similar polymers or two or more chemically distinct polymers. In one set of embodiments, the elastic polymeric component comprises an isocyanate cross-linked polyurethane generated from low molecular weight monomers such as polycaprolactone. In some embodiments, the low molecular weight monomers comprise one or more hydroxyl functional groups (e.g., a diol, a triol).

In some embodiments, the polymeric component may comprise a polymer that has a relatively large elastic modulus. In some cases, the polymer may have an elastic modulus of at least 0.01 GPa, at least 0.05 GPa, at least 0.1 GPa, at least 1 GPa, at least 5 GPa, at least 10 GPa, at least 15 GPa, at least 20 GPa, at least 25 GPa, or at least 30 GPa. In some cases, the polymer may have an elastic modulus of less than or equal to 35 GPa, less than or equal to 30 GPa, less than or equal to 25 GPa, less than or equal to 20 GPa, less than or equal to 15 GPa, less than or equal to 10 GPa, less than or equal to 5 GPa, less than or equal to 1 GPa, less than or equal to 0.1 GPa, or less than or equal to 0.05 GPa. Any of the above-referenced ranges may be possible (e.g., at least 0.01 GPa and less than or equal to 35 GPa). Other ranges are also possible. Examples of such polymer include, but are not limited to polycarbonate, polymethyl methacrylate, polystyrene, etc. In an exemplary embodiment, the polymeric component comprises polycarbonate. In some embodiments, the polymeric component has particular mechanical properties such that the polymeric component resists brittle breakage but is sufficiently stiff such that it may withstand internal physiological pressure and/or maintain residence of the structure. The polymeric component may be made of a single material, essentially a single material, or with a plurality of materials including the various materials already discussed, or a reinforcing material, a fiber, a wire, a braided material, braided wire, braided plastic fibers.

In certain embodiments, the one or more microfluidic channel (e.g., microfluidic channel 16 in FIGS. 1-2) of the article has a particular average cross-sectional dimension. The “cross-sectional dimension” (e.g., a width, a height, a radius) of the channel is measured perpendicular to the direction of fluid flow. In some embodiments, the average cross-sectional dimension of one or more microfluidic channels is less than or equal to 1 mm, less than or equal to 800 microns, less than or equal to 600 microns, less than or equal to 500 microns, less than or equal to 400 microns, less than or equal to 300 microns, less than or equal to 200 microns, less than or equal to 100 microns, less than or equal to 50 microns, less than or equal to 25 microns, less than or equal to 20 microns, less than or equal to 15 microns, or less than or equal to 10 microns. In certain embodiments, the average cross-sectional dimension of the microfluidic channel is greater than or equal to 5 microns, greater than or equal to 10 microns, greater than or equal to 15 microns, greater than or equal to 20 microns, greater than or equal to 25 microns, greater than or equal to 50 microns, greater than or equal to 100 microns, greater than or equal to 200 microns, greater than or equal to 300 microns, greater than or equal to 400 microns, greater than or equal to 500 microns, greater than or equal to 600 microns, greater than or equal to 800 microns, or greater than or equal to 1 mm. Combinations of the above-referenced ranges are also possible (e.g., greater than or equal to 5 microns and less than or equal to 1 mm). Other ranges are also possible.

The one or more microfluidic channels (e.g., microfluidic channel 16 in FIGS. 1-2) may have any of a variety of appropriate largest dimensions (e.g., M in FIG. 1A). In some embodiments, the microfluidic channels may have a largest dimension of at least 2 cm, at least 5 cm, at least 7.5 cm, at least 0.1 m, at least 0.25 m, at least 0.5 m, at least 0.75 m, at least 1 m, at least 1.25 m, at least 1.5 m, at least 1.75 m, at least 2 mm, or at least 2.5 mm. In some embodiments, the microfluidic channel may have a largest dimension of less than or equal to 3 m, less than or equal to 2.5 m, less than or equal to 2 m, less than or equal to 1.75 m, less than or equal to 1.5 m, less than or equal to 1.25 m, less than or equal to 1 m, less than or equal to 0.75 m, less than or equal to 0.5 m, less than or equal to 0.25 m, less than or equal to 0.1 m, less than or equal to 7.5 cm, or less than or equal to 5 cm. Any of the above-referenced ranges may be possible (e.g., at least 2 cm and less than or equal to 3 m). Other ranges are also possible.

The one or more microfluidic channels (e.g., microfluidic channel 16 in FIGS. 1-2) may have any of a variety of appropriate aspect ratios. The one or more microfluidic channels may have an aspect ratio of at least 20:1, at least 25:1, at least 50:1, at least 75:1, at least 100:1, at least 250:1, at least 500:1, at least 1,000:1, at least 2,500:1, at least 5,000:1, at least 10,000:1, at least 20,000:1, or at least 40,000:1. In some embodiments, the one or more microfluidic channels may have an aspect ratio of less than or equal to 60,000:1, less than or equal to 40,000:1, less than or equal to 20,000:1, less than or equal to 10,000:1, less than or equal to 5,000:1, or less than or equal to 2,500:1, less than or equal to 1,000:1, less than or equal to 500:1, less than or equal to 250:1, less than or equal to 100:1, less than or equal to 75:1, less than or equal to 50:1, or less than or equal to 25:1. Combination of the above-referenced ranges are possible (e.g., at least 20:1 and less than or equal to 60,000:1). Other ranges are also possible.

In one set of embodiments, the microfluidic channel may house one or more therapeutics and/or chemicals. In some embodiments, the chemical comprises a neurochemical. In some embodiments, the neurochemical is capable of affecting the myenteric plexus. Examples of chemicals and/or neurochemical include, but are not limited to nitric oxide, glucagon, neuro-inhibitors, baclofen.

The microfluidic channel may house any of a variety of therapeutics, agents, and/or active ingredients.

According to some embodiments, the articles described herein are compatible with one or more therapeutic, diagnostic, and/or enhancement agents, such as drugs, nutrients, microorganisms, in vivo sensors, and tracers. In some embodiments, the active substance, is a therapeutic, nutraceutical, prophylactic or diagnostic agent. While much of the specification describes the use of therapeutic agents, other agents listed herein are also possible.

For example, agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals.

Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro)viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In certain embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In certain embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery article. These may be vitamins, supplements such as calcium or biotin, or natural ingredients such as plant extracts or phytohormones.

In another embodiment, the therapeutic agent is an immunosuppressive agent. Exemplary immunosuppressive agents include glucocorticoids, cytostatics (such as alkylating agents, antimetabolites, and cytotoxic antibodies), antibodies (such as those directed against T-cell receptors or 11-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In some embodiments, the therapeutic agent is a small molecule drug having molecular weight less than about 2500 Daltons, less than about 2000 Daltons, less than about 1500 Daltons, less than about 1000 Daltons, less than about 750 Daltons, less than about 500 Daltons, less or than about 400 Daltons. In some cases, the therapeutic agent is a small molecule drug having molecular weight between 200 Daltons and 400 Daltons, between 400 Daltons and 1000 Daltons, or between 500 Daltons and 2500 Daltons.

In certain embodiments, the therapeutic agent is present in the article in an amount greater than or equal to 1 gram, greater than or equal to 2 grams, greater than or equal to 3 grams, greater than or equal to 5 grams, greater than or equal to 10 grams, greater than or equal to 20 grams, greater than or equal to 30 grams, greater than or equal to 40 grams, greater than or equal to 50 grams, greater than or equal to 60 grams, greater than or equal to 70 grams, or greater than or equal to 80 grams, greater than or equal to 90 grams. In some embodiments, the therapeutic agent is present in the residence article in an amount of less than or equal to 100 grams, less than or equal to 90 grams, less than or equal to 80 grams, less than or equal to 70 grams, less than or equal to 60 grams, less than or equal to 50 grams, less than or equal to 40 grams, less than or equal to 30 grams, less than or equal to 20 grams, less than or equal to 10 grams, less than or equal to 5 grams, less than or equal to 3 grams, or less than or equal to 2 grams. Combinations of the above-referenced ranges are also possible (e.g., greater than or equal to 1 gram and less than or equal to 100 grams, greater than or equal to 2 grams and less than or equal to 100 grams, greater than or equal to 3 grams and less than or equal to 100 grams). Other ranges are also possible.

In some embodiments, the articles described herein comprises two or more types of therapeutic agents. For example, in some embodiments, a first therapeutic agent and a second therapeutic agent are present in the residence article such that the total amount of the first and second therapeutic agent is in one or more ranges described above (e.g., the total amount of therapeutic agent is greater than or equal to 1 gram and less than or equal to 100 grams). In some embodiments, each therapeutic agent is present in an amount such that the total amount of therapeutic agents is greater than or equal to 1 gram. In some embodiments, each therapeutic agent is present in an amount as described above (e.g., each therapeutic agent is present in an amount of greater than or equal to 1 gram and less than or equal to 100 grams).

In certain embodiments, the therapeutic agent is present in the residence article at a concentration such that, upon release from the residence article, the therapeutic agent elicits a therapeutic response.

In some embodiments, a subject may demonstrate health benefits, e.g., upon administration of the residence article.

In some embodiments, a combination of chemicals or therapeutics may be released from the one or more microfluidic channels, e.g., such that the combination may achieve a synergistic therapeutic effect. Alternatively, the combination of chemicals may have an antagonistic effect on each other, e.g., where one or more chemical(s) in the combination may be an antidote and/or reversal agent for the other chemical(s). In some embodiments, the chemicals and/or therapeutic agents stored in the microfluidic channels may be in an inactive state during storage, and transform into an active state upon release from the microfluidic channels. In some embodiments, the release of the chemicals and/or therapeutics from the microfluidic channels may occur via electrically and/or optically stimulation.

In some cases, prior to the release, the combination of chemicals and/or therapeutics may be stored together in the same microfluidic channel(s). Alternatively, each of the combination may be stored separately in different microfluidic channels prior to the release. Each of the one or more microfluidics channels may be separately controlled, e.g., by the controller described herein, to release the chemical or therapeutics. Parameters such as flow rate, release time, duration of release, etc., of chemicals stored in each of the microfluidic channels may be also controlled separately. For example, in one set of embodiments, one or more chemicals and/or therapeutic agents (e.g., one or more antibiotics) may be released from the one or more microfluidic channels and delivered to location (e.g., GI tract) internal the subject in a chronic fashion.

In certain embodiments, the one or more electrodes may comprise a coating on at least a portion of an outer surface of the one or more electrodes. In certain embodiments, the coating comprises one or more of a polymer described herein with respect to the polymeric component. Additional non-limiting examples of suitable polymers for the coating include perfluoroalkoxy alkane, polyimide, polyester, polyethylene, polyvinylchloride, polyamide, polybutylene terephthalate, thermoplastic elastomers, ethylene propylene copolymers, polypropylene, fluoropolymers.

The article, devices, and/or systems described herein may be employed to treat any of a variety of GI motility disorders. In some embodiments, the GI motility disorder comprises esophageal motility disorders selected from the group of gastroesophageal reflux disease (GERD), achalasia, jackhammer esophagus, absent peristalsis, and upper esophageal sphincter/lower esophageal sphincter (UES/LES) dysfunction. Non-limiting examples of motility disorders in the stomach include functional dyspepsia, gastroparesis, etc.

Certain aspects of the disclosure are directed to an implantation tool and interrelated methods for implantation (e.g., submucosal or intramuscular implantation) of an article, e.g., such as article 10 as illustrated in FIGS. 1-2.

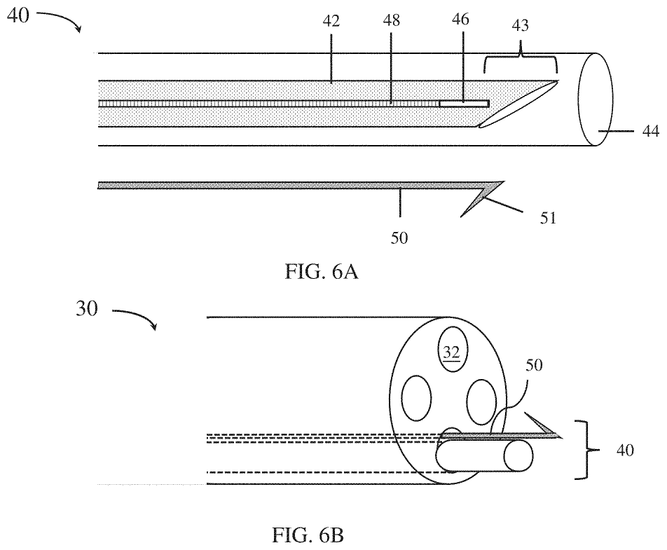

In some embodiments, the implantation tool comprises an incision needle configured for incision of a tissue or lumen. In some embodiments, the incision needle is a hollow needle comprising a tip. A non-limiting representation of the implantation tool is shown in FIG. 6A. As shown, implantation tool 40 comprises incision needle 42 (e.g., a hollow needle) having tip 43.

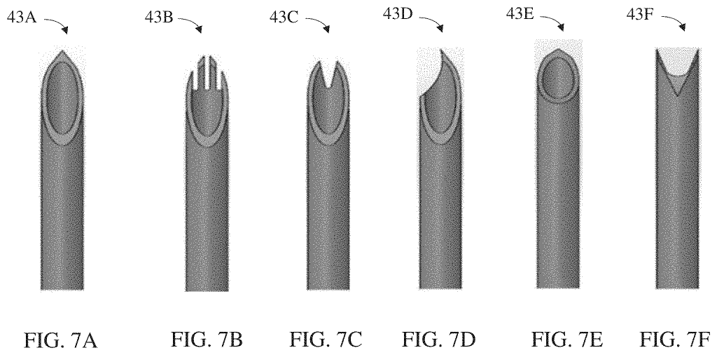

In some embodiments, the tip of the incision needle (e.g., hollow needle) may have a certain shape, angle, and/or associated components that impart the needle with enhanced capabilities. For example, compared to a standard needle tip, the tips described herein may allow for low-force incision and penetration, prevention of slipping, identification of a specific tissue layer, and/or reduced perforation at the mucosa or lumen. In one set of embodiments, the tip may have a particular shape (e.g., a curved, grooved, pitchfork, broad, or dual point shape) that allows for low-force incision and penetration, prevention of slipping, and/or reduced perforation at the mucosa or lumen. For example, as illustrated in FIGS. 7A-7F, the hollow needle may have a tip that differ from a standard tip 43A, such as a grooved tip 43B, a pitchfork tip 43C, a curved tip 43D, a broad base tip 43E, or a dual point tip 43F.

Additionally or alternatively, the tip of the incision needle (e.g., hollow needle) may have an angle that allows for low-force incision and penetration, prevention of slipping, and/or reduced perforation at the mucosa or lumen. For example, the tip of the incision needle may be a tissue-penetrating tip having a particular angle relative to a base (e.g., a horizontal plane) of the needle. In some embodiments, the tip may have an angle relative to the base of the needle of greater than or equal to 20°, greater than or equal to 25°, greater than or equal to 30°, greater than or equal to 35°, greater than or equal to 40°, greater than or equal to 50°, greater than or equal to 60°, greater than or equal to 70°, or greater than or equal to 80°. In some embodiments, the tip may have an angle relative to a base of the needle of less than or equal to 90°, less than or equal to 80°, less than or equal to 70°, less than or equal to 60°, less than or equal to 50°, less than or equal to 40°, less than or equal to 35°, less than or equal to 30°, or less than or equal to 25°. Any of the above-referenced ranges are possible (e.g., greater than or equal to 20° and less than or equal to)90°. Other ranges are also possible.

In some embodiments, the implantation tool comprises an overtube (i.e., a sheath) associated with (e.g., coupled to, encapsulates, etc.) the incision needle (e.g., a hollow needle). For instance, in one set of embodiments, the overtube is a hollow tube or cylinder that encapsulates or houses the incision needle. For example, as illustrated in FIG. 6A, overtube 44 may be employed to house incision needle 42. The overtube may comprise any appropriate material (e.g., a polymer described herein) and may be elastic in nature.

In some embodiments, the overtube is adapted and designed to receive an article configured for submucosal implantation within a lumen of a subject. For example, the overtube may be configured to receive the article described herein (e.g., article 10 in FIGS. 1A-1E) for submucosal implantation within a lumen (e.g., GI tract) of a subject. The overtube may be sized such that it allows insertion of the article.

In some embodiments, the implantation tool comprises an impedance sensor. In some cases, the impedance sensor may be associated with one or more components of the implantation tool, e.g., such as the overtube, the incision needle, and/or another associated component. For example, as illustrated in FIG. 6A, the implantation tool may comprise impedance sensor 46 associated with (e.g., coupled with, disposed in, etc.) incision needle 42.

In some embodiments, the implantation tool comprises a guidewire associated with the incision needle (e.g., a hollow needle). In some cases, the guidewire and/or part of the guidewire may act as the impedance sensor. For example, as illustrated in FIG. 6A, implantation tool 40 comprises guidewire 48 associated with (e.g., contained within) incision needle 42. In some cases, the tip portion of guidewire 48 may act as impedance sensor 46. In some embodiments, via the impedance sensor, the location of the needle tip in the tissue during implantation may be determined based on a measured impedance value. In some embodiments, in addition to functioning as an impedance sensor, the guidewire may be configured to facilitate dissection of a tissue and/or serve as a marker for imaging purposes.

While FIG. 6A illustrates an embodiment in which the implantation tool comprises an impedance sensor, it should be noted any appropriate types of sensors may be employed, including but limited to, temperature sensors, pressure sensors, position sensors, etc.

In some embodiments, the implantation tool may optionally comprise a hook. In some such embodiments, the hook may be associated with (e.g., coupled to, adjacent, etc.) the incision needle (e.g., a hollow needle) and/or associated components. The term “associated,” as used herein, means generally held in close proximity, for example, a hook that is associated with the incision needle may be adjacent to the incision needle. As used herein, when hook is referred to as being “adjacent” the needle, it can be directly adjacent to (e.g., in contact with) the needle, or one or more intervening components also may be present.

For example, as illustrated in FIG. 6A, implantation tool 40 comprises hook 50 associated with (e.g., adjacent) incision needle 42 and/or associated components (e.g., overtube 44). In some embodiments, the hook is a self-expanding hook. That is, the hook may be capable of adhering onto a surface of a tissue or lumen upon contact and/or exert a tension on the contacted surface. In some cases, as described in more detail below, the hook may advantageously adhere onto a surface of a tissue or lumen and facilitate incision of the needle into a lumen.

In some embodiments, the hook may have a tip having a certain shape that is advantageous for adhering onto the surface of a tissue or lumen. For instance, as illustrated in FIG. 6A, hook 51 may comprise a beveled tip bent at a particular angle. In some embodiments, the beveled tip may have an angle of at least 25 degrees, at least 30 degrees, at least 35 degrees, at least 40 degrees, 45 degrees, at least 50 degrees, at least 55 degrees, at least 60 degrees, at least 65 degrees, at least 70 degrees, or at least 75 degrees. In some embodiments, the beveled tip may have an angle of less than or equal to 80 degrees, less than or equal to 75 degrees, less than or equal to 70 degrees, less than or equal to 65 degrees, less than or equal to 60 degrees, less than or equal to 55 degrees, less than or equal to 50 degrees, less than or equal to 45 degrees, less than or equal to 40 degrees, less than or equal to 35 degrees, or less than or equal to 30 degrees. Any of the above-referenced ranges are possible (e.g., at least 25 degrees and less than or equal to 90 degrees, at least 45 degrees and less than or equal to 90 degrees). Other ranges are also possible.

The hook may comprise any of a variety of materials. In some embodiments, the hook comprises a metal alloy. Non-limiting of materials for forming the hook may include nitinol, stainless steel, Beta titanium alloys, Beta brass, nickel aluminum, etc.

In some embodiments, the implantation tool may be configured such that it may be inserted into an endoscope channel. For example, as shown in FIG. 6B, an endoscope 30 comprises a plurality of endoscope channels 32, and implantation tool 40 (from FIG. 6A) may be configured such that it can be inserted into endoscope channel 32.

The implantation tool and associated components (e.g., overtube, hooks, etc.) may have any of a variety of appropriate sizes, shapes, and/or properties, as described in more detail below.

The implantation tool described herein may be employed for implantation into any of a variety of regions internal a subject. Non-limiting examples of such regions include the mucosal layer, submucosal layer, intramuscular layer, serosal layer, peritoneum layer, etc. The implantation tool may advantageously comprise one or more components (e.g., an impedance sensor, etc.) capable of identifying or distinguish the one or more of the above-reference regions.

Certain aspects of the disclosure are directed to methods of submucosal or intramuscular implantation of an article, device, and/or system using an implantation tool. For example, one such methods may be directed to submucosal and/or intramuscular implantation of an article and/or system (e.g., article 10 and/or system 20 in FIGS. 1-2) described herein using an implantation tool (e.g., implantation tool 40 in FIGS. 6A-6B). However, it should be noted that the implantation method may be employed for implantation of any article, device, and/or system, and one of ordinary skill in the art would understand, based upon the teachings of this specification, that such implantation methods are not limited solely to the article and/or system described herein.

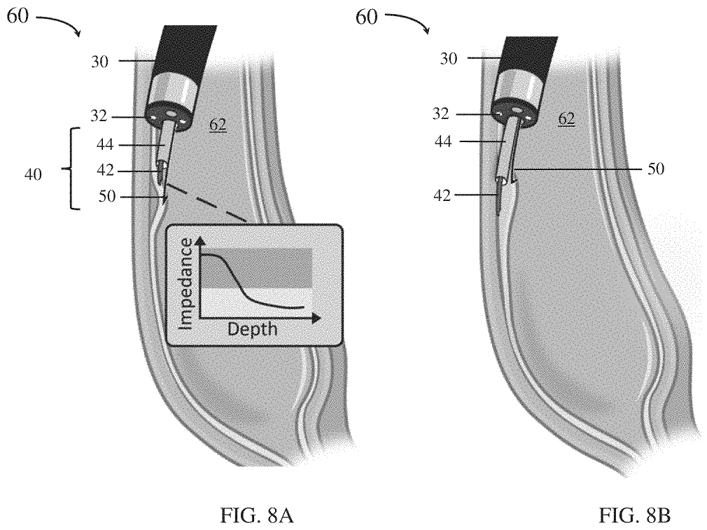

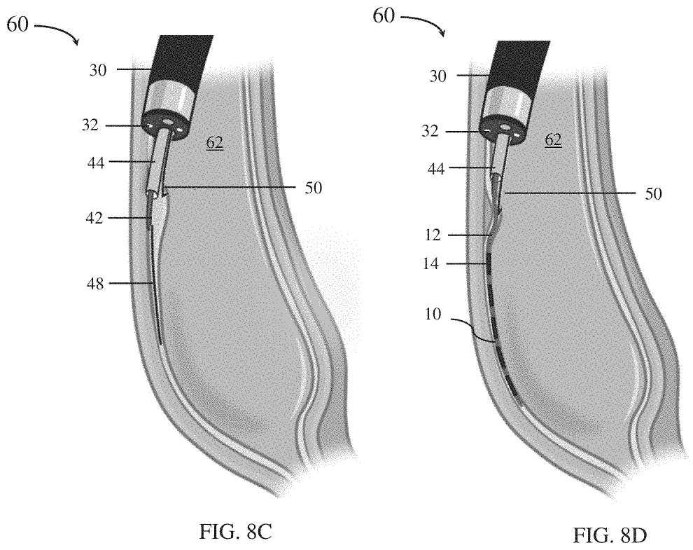

In some embodiments, a method of submucosal or intramuscular implantation comprises inserting an incision needle at a lumen internal to a subject. In some embodiments, the incision needle may be a hollow needle (e.g., as shown in FIG. 6A) and may be a part of the implantation tool described herein (e.g., implantation tool 40). For example, as shown in FIG. 8A, in system 60, an incision needle 42 may be inserted at lumen 62 internal to a subject. The incision needle may be inserted at any of a variety of locations, e.g., a lumen in the GI tract (e.g., esophagus, stomach, small intestine, and/or large intestine). For instance, as illustrated in FIG. 8A, incision needle 42 may be inserted at a lumen of the esophagus.

The incision needle may be delivered to the lumen via any appropriate method. In some embodiments, prior to inserting the incision needle at the lumen, the incision needle may be delivered to the lumen endoscopically. For example, the incision needle may be delivered to the lumen via insertion through an endoscope channel of an endoscope. For instance, as shown in FIG. 8A, incision needle 42 may be inserted through endoscope channel 32 of endoscope 30.

In some cases, the incision needle may be contained within an overtube (e.g., overtube 44 in FIG. 7A), such that the incision needle may be protected from undesirable deformation (e.g., damage of needle tip, etc.) while passing through the endoscope channel. For example, as shown in FIG. 8A, incision needle 42 may be contained within overtube 44. In some such cases, incision needle 42 and overtube 44 may be co-delivered to lumen 42, after which incision needle 42 may be inserted into lumen 62.

In some embodiments, the incision needle may have a certain set of properties and/or associated components that allow for efficient insertion of the needle into the lumen. For example, such a set of properties and/or associated components may include, but are not limited to, shape of the incision needle tip, angle of incision at the lumen, an associated hook, etc. In some instances, such a set of properties and/or associated component may advantageously correlate to a relatively low incision or penetration force. For example, as mentioned above, the incision needle tip may have a particularly advantageous design and/or shape (e.g., a curved, grooved, pitchfork, broad, or dual point shape as shown in FIGS. 7B-7F).

In some cases, it may be particularly advantageous to insert the incision needle (e.g., tip of the incision needle) at a certain angle relative to a surface of a lumen or tissue. In some such cases, employing a particular insertion angle may allow for low-force incision and penetration, prevention of slipping, and/or reduced perforation at the mucosa or lumen. In some embodiments, the incision angle relative to a surface of a lumen or tissue may be greater than or equal to 20°, greater than or equal to 25°, greater than or equal to 30°, greater than or equal to 35°, greater than or equal to 40°, greater than or equal to 50°, greater than or equal to 60°, greater than or equal to 70°, or greater than or equal to 80°. In some embodiments, the incision angle relative to a surface of a lumen or tissue may be less than or equal to 90°, less than or equal to 80°, less than or equal to 70°, less than or equal to 60°, less than or equal to 50°, less than or equal to 40°, less than or equal to 35°, less than or equal to 30°, or less than or equal to 25°. Any of the above-referenced ranges are possible (e.g., greater than or equal to 20° and less than or equal to) 90°. Other ranges are also possible.

In some embodiments, the incision needle (e.g., hollow needle 42 in FIG. 6A) may be associated with a relatively low incision force when inserted into the lumen at an incision angle described above. For example, in some embodiments, the incision force of the needle at the incision angle described herein (e.g., an angle of about 20 degrees) relative to the lumen is less than or equal to 5 mN, less than or equal to 4.5 mN, less than or equal to 4 mN, less than or equal to 3.5 mM, less than or equal to 3 mM, less than or equal to 2.5 mN, less than or equal to 2 mN, less than or equal to 1.5 mM, or less than or equal to 1 mN. In some embodiments, the incision force of the needle at a particular angle (e.g., an angle of about 20 degrees) relative to the lumen is greater than or equal to 0.5 mN, greater than or equal to 1 mN, greater than or equal to 1.5 mN, greater than or equal to 2 mM, greater than or equal to 2.5 mM, greater than or equal to 3 mN, greater than or equal to 3.5 mN, greater than or equal to 4 mM, or greater than or equal to 4.5 mN. Any of the above-referenced ranges are possible (e.g., greater than or equal to 0.5 mN and less than or equal to 5 mN, or greater than or equal to 1 mN and less than or equal to 3 mN). Other ranges are also possible.

Certain embodiments comprise adhering a hook associated with the incision needle on a wall of the lumen prior to inserting the incision needle at the lumen. In some cases, adherence of a hook on the wall of the lumen may advantageously facilitate insertion of the incision needle into the lumen. For example, as illustrated in FIG. 8A, hook 50 associated with the incision needle may be inserted through overtube 44 and adhered onto a wall of lumen 62. As hook 62 adheres onto the wall of lumen 62, incision needle 42 may be inserted into lumen 42. As mentioned above, hook 50 may be a part of an implantation tool (e.g., implantation tool 40 in FIG. 6A) and may have any of a variety of properties described herein, e.g., such as a beveled tip.

In some embodiments, the method of submucosal or intramuscular implantation comprises localizing a submucosal layer based on a sensed parameter. For example, as illustrated in FIG. 8B, incision needle 42 may advance through the layers of tissue until localizing a submucosal layer based on a sensed parameter.

In one set of embodiments, the sensed parameter may be an impedance measurement obtained from an impedance sensor (e.g., impedance sensor 46) associated with the incision needle (e.g., hollow needle 42) or associated components. Without wishing to be bound by theories, it is believed that impedance measurements may vary as a function of the depth of tissue incision (e.g., as shown in FIG. 8A), and it may thus be possible to localize and identify a submucosal or intramuscular layer based such impedance measurements. As mentioned above, the impedance sensor may be a (or part of a) guidewire (e.g., guidewire 42 in FIG. 6A).

It should be understood that the sensor used to localize submucosal and/or intramuscular layer is not limited an impedance sensor, and that any appropriate sensors may be employed, as long as a parameter sensed by the sensor can be used to localize the submucosal and or intramuscular layer.

In some embodiments, the step of localizing a submucosal or intramuscular layer based on a sensed parameter comprises sensing an impedance value associated with said layer(s). In some embodiments, the sensed impedance value associated with the submucosal or intramuscular layer may be less than or equal to 1,000 kOhm·s, less than or equal to 700 kOhm·s, less than or equal to 500 kOhm·s, less than or equal to 400 kOhm·s, less than or equal to 300 kOhm·s, less than or equal to 200 kOhm·s, less than or equal to 100 kOhm·s, less than or equal to 70 kOhm·s, less than or equal to 50 kOhm·s, less than or equal to 30 kOhm·s, or less than or equal to 20 kOhm·s. In some embodiments, the sensed impedance value associated with the submucosal or intramuscular layer may be greater than or equal to 10 kOhm·s, greater than or equal to 20 kOhm·s, greater than or equal to 30 kOhm·s, greater than or equal to 50 kOhm·s, greater than or equal to 70 kOhm·s, greater than or equal to 100 kOhm·s, greater than or equal to 200 kOhm·s, greater than or equal to 300 kOhm·s, greater than or equal to 400 kOhm·s, greater than or equal to 500 kOhm·s, or greater than or equal to 700 kOhm·s. Any of the above-referenced values are possible (e.g., greater than or equal to 10 kOhm·s and less than or equal to 1000 kOhm·s, greater than or equal to 100 kOhm·s and less than or equal to 600 kOhm·s, or greater than or equal to 300 kOhm·s and less than or equal to 700 kOhm·s). Other ranges are also possible.

In some embodiments, upon localizing a submucosal or intramuscular layer, an article may be implanted via the incision needle (and/or associated component) in the submucosal or intramuscular layer of the subject. For example, in some cases, an associated component, e.g., a guidewire, may be employed to facilitate implantation of the article. In one set of embodiments, a guidewire (e.g., guidewire 48 in FIG. 6A) associated with the incision needle (e.g., hollow needle 42 in FIG. 6A) may be optionally employed to separate (e.g., dissect) the submucosal of intramuscular layer from the lumen. For example, as illustrated in FIG. 8C, guidewire 48 associated with needle 42 may be employed to separate the submucosal or intramuscular layer from lumen 62. In some embodiments, the step of separating the submucosal or intramuscular layer from the lumen comprises passing the guidewire (e.g., guidewire 48 in FIG. 8C) along a curvature of the submucosal or intramuscular layer of an organ (e.g., esophagus).

A non-limiting representation of the step of implanting an article (e.g., article 10) is illustrated in FIG. 8D. As shown, via incision needle 42 and/or associated components (e.g., guidewire 48), article 10 may be implanted in the submucosal or intramuscular layer of the subject. In some embodiments, the step of implanting comprising inserting the article along a curvature of the submucosal or intramuscular layer of an organ. For example, as shown in FIG. 8D, article 10 may be inserted along a curvature of the submucosal or intramuscular layer of the esophagus.

In some embodiments, the implanted article may be an article described herein (e.g., article 10 in FIGS. 1-2). The implanted article may have any of the properties, components, and/or arrangements described with respect to article 10 in FIGS. 1-2. For example, as shown in FIG. 8D, implanted article 10 may comprise a polymeric component 12, one or more electrodes 14 disposed within the polymeric component 12, and a microfluidic channel (not shown) disposed within the polymeric component.

While FIGS. 8A-8D are directed to a method of implanting a device into a submucosal or intramuscular layer in a subject, it should be understood that the disclosure is not so limited, and that in certain embodiments, the same method may be employed for implanting a device into other locations, e.g., a mucosal layer, serosal layer, or a peritoneum layer.

In some embodiments, the overtube or sheath (e.g., overtube 44 in FIGS. 6A and 8A) may have any suitable shape or geometry, e.g., that may be useful for allowing the overtube to be inserted into a typical endoscope channel. The overtube may have the shape of a cylinder, a rectangular prism, a cone, etc., but is not limited to these shapes. In some embodiments, the overtube may have a cross-sectional dimension, e.g. a diameter, of at least 2 mm, at least 2.2 mm, at least 2.4 mm, at least 2.8 mm, at least 3 mm, at least 3.4 mm, at least 3.8 mm, or at least 4 mm. In some embodiments, the guidewire may have a cross-sectional dimension of less than or equal to 5 mm, less than or equal to 4 mm, less than or equal to 3.8 mm, less than or equal to 3.4 mm, less than or equal to 3 mm, less than or equal to 2.8 mm, less than or equal to 2.4 mm, or less than or equal to 2.2 mm. Combinations of these ranges are possible (at least 2 mm and less than or equal to 5 mm, or at least 2 mm and less than or equal to 2.8 mm). Other ranges of width may be possible.

The overtube (e.g., overtube 44 in FIG. 6A) may comprises any of a variety of common polymers and/or plastics described herein. The overtube may comprise a polymer or plastic that is elastic, biocompatible, and/or has non-stick properties. For example, the overtube may comprise any of a variety of polymers described herein with respect to the polymeric component (e.g., polymeric component 12 in FIGS. 1-2). In some cases, the overtube may comprises a non-stick polymeric material such as PTFE.

In some embodiments, the incision needle (e.g., incision needle 42 in FIGS. 6A and 8A) may have any suitable shape or geometry, e.g., that may be useful for incision into a lumen. In some case, the incision needle may be sized such that it may be inserted into the overtube (e.g., overtube 44 in FIGS. 6A and 8A). In some embodiments, the incision needle (e.g., a hollow needle) may have a cross-sectional dimension, e.g. an outer diameter, of at least 0.1 mm, at least 0.2 mm, at least 0.4 mm, at least 0.6 mm, at least 0.8 mm, at least 1 mm, at least 1.25 mm, at least 1.5 mm, or at least 1.75 mm. In some embodiments, the incision needle may have a cross-sectional dimension of less than or equal to 2 mm, less than or equal to 1.75 mm, less than or equal to 1.5 mm, less than or equal to 1.25 mm, less than or equal to 1 mm, less than or equal to 0.8 mm, less than or equal to 0.6 mm, less than or equal to 0.4 mm, less than or equal to 0.2 mm. Combinations of these ranges are possible (at least 0.1 mm and less than or equal to 2 mm). Other ranges may be possible.

In some embodiments, the guidewire (e.g., guidewire 48 in FIGS. 6A and 8C) may have any suitable shape or geometry, e.g., that may be useful for allowing the guidewire to be inserted into a target location (e.g., a submucosal or intramuscular layer) of a subject. In some embodiments, the guidewire may be sized such that it may be configured to separate the submucosal (or intramuscular layer) from the lumen. For instance, the guidewire may have the shape of a cylinder, a rectangular prism, a cone, etc., but is not limited to these shapes. In some embodiments, the guidewire may have a cross-sectional dimension, e.g. a diameter, of at least 100 micrometers, at least 200 micrometers, at least 300 micrometers, at least 400 micrometers, at least 500 micrometers, at least 600 micrometers, at least 700 micrometers, or at least 800 micrometers. In some embodiments, the guidewire may have a cross-sectional dimension of less than or equal to 1000 micrometers, less than or equal to 800 micrometers, less than or equal to 700 micrometers, less than or equal to 600 micrometers, less than or equal to 500 micrometers, less than or equal to 400 micrometers, less than or equal to 300 micrometers, or less than or equal to 200 micrometers. Combinations of these ranges are possible (between 100 micrometers and 1000 micrometers) Other ranges may be possible.

In some embodiments, the guidewire may be configured to allow insertion into a location having a certain dimension and/or tortuosity. For example as shown in FIG. 8C, guidewire 48 may be inserted along a curvature of the submucosal or intramuscular layer. In some such embodiments, the guidewire may have a certain elasticity and stiffness that allows for conformation to the curvature.

The guidewire may have any of a variety of dimensions. In some embodiments, the guidewire may have a largest dimension compared to a largest dimension of the implanted article. For example, the guidewire may have a largest dimension (e.g., length) in one or more of the ranges described with respective to the largest dimension of article 10 in FIGS. 1A-1E.

In some embodiments, the residence article (e.g., article 10 and/or implantation tool 40) is administered such that the residence article enters the stomach of the subject and is retained in the stomach for a residence period (e.g., of greater than or equal to 24 hours). The residence article may be configured to transmit a signal from the residence article to a device external (e.g., extracorporeal) of the stomach and/or configured to receive a signal from a device external (e.g., extracorporeal) of the stomach. For example, in some embodiments, the residence article may be configured to transmit and/or receive physiological conditions about the subject such as e.g., temperature (e.g., gastric internal temperature), pH, pressure, or other biophysical characteristics. For example, the residence article may comprise (and/or be in electronic communication with) one or more sensors configured to determine one or more physiological conditions about the subject. In some embodiments, the residence article (e.g., article 10 and/or implantation tool 40) comprises one or more sensors (e.g., an impedance sensor, a biomolecular sensor, a gas sensor, a temperature sensor, a pressure sensor, a motion sensor, an accelerometer, a pH sensor, a biochemical sensor), a wireless identification microchip, and/or an imaging system (e.g., a camera). In some embodiments, the residence article (e.g., article 10 and/or implantation tool 40) is configured to generate and/or receive a signal (e.g., a wireless signal). In some embodiments, the signal triggers the residence article (e.g., article 10) to release a pharmaceutical agent from the residence article. In some embodiments, the signal provides a physiological condition of the subject to the device external of the stomach. In some embodiments, the signal mediates the exit of the residence article (e.g., article 10) from the stomach through the pylorus, as described herein.

In some embodiments, the residence article (e.g., article 10 in FIGS. 1-2 or implantation tool 40 in FIGS. 6A-6B) is associated with and/or comprises a power source. The power source may include any appropriate material(s), such as one or more batteries, photovoltaic cells, etc. Non-limiting examples of suitable batteries include Li-polymer (e.g., with between 100 and 1000 mAh of battery life), Li-ion, nickel cadmium, nickel metal hydride, silver oxide, or the like. In some cases, the battery may apply a voltage (e.g., to a degradable material as described herein) in response to a physiological and/or external metric and/or signal (e.g., by a user). For example, the voltage may be used to trigger the exit of the residence article by e.g., applying a voltage to thermally sensitive degradable component as described herein. For example, the average magnitude of the voltage applied to the degradable component(s) may be between 0.001 to 0.01 V, between 0.01 to 0.1 V, between 0.1 V and 10.0 V, between 1.0 V and 8.0 V, between 2.0 V and 5.0 V, between 0.1 V and 5.0 V, between 0.1 V and 1.5 V, between 0.1 V and 1.0 V, between 1.0 V and 3.0 V, between 3.0 V and 8.0 V, or any other appropriate range.

In this respect, it should be appreciated that one implementation of the embodiments described herein may, in some cases, comprise at least one computer-readable storage medium (e.g., RAM, ROM, EEPROM, flash memory or other memory technology, or other tangible, non-transitory computer-readable storage medium) encoded with a computer program (i.e., a plurality of executable instructions) that, when executed on one or more processors, performs the above-discussed functions of one or more embodiments. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Any residence article circuitry may be implemented by any suitable type of analog and/or digital circuitry. For example, the residence article circuitry may be implemented using hardware or a combination of hardware and software. When implemented using software, suitable software code can be executed on any suitable processor (e.g., a microprocessor) or collection of processors. The one or more residence articles can be implemented in numerous ways, such as with dedicated hardware, or with general purpose hardware (e.g., one or more processors) that is programmed using microcode or software to perform the functions recited above.

The following examples are intended to illustrate certain embodiments of the present invention, but do not exemplify the full scope of the invention.

### EXAMPLE 1

The following example describes an implantation tool configured for submucosal delivery of an article, according to certain embodiments.

### 1. Introduction

Gastrointestinal (GI) dysmotility and associated conditions are common. Current therapies including pharmacological, behavioral and surgical approaches are however limited in their efficacy. Targeted interventions addressing the underlying neuromuscular pathology with electrical neuromuscular stimulation stands to transform our capacity to more effectively treat dysmotility. Here, a minimally invasive implantation tool was developed to endoscopically penetrate the mucosa, accurately localize the submucosa, and safely deploy a device to directly interface with the myenteric nervous system. An implantable, closed-loop myenteric neuroprosthesis was developed to activate or relax myenteric musculature through electrochemical stimulation in response to sensed food stimuli. The neuroprosthesis allowed generation of coordinated peristaltic waves, which manifested in a significant increase in the motility rate (p<0.05, student's t-test) in a swine model of esophageal and stomach dysmotility. Further, by directly neuromodulating the myenteric plexus mimicking meal ingestion, peristalsis was induced in a fasted state and a metabolic response commensurate with a fed state was achieved, thereby yielding an illusory sense of metabolic satiety. This implantation platform and neuroprosthesis expanded opportunities in fundamental studies and treatments of metabolic and neuromuscular pathologies of the GI tract.

Gastrointestinal (GI) motility is generally orchestrated by extrinsic autonomic neural control (sympathetic and parasympathetic pathways) which yield peristaltic waves that churn and propagate food through the tract. Motility disorders of the esophagus (gastroesophageal reflux disease (GERD), achalasia, and dysphagia) and stomach (functional dyspepsia, gastroparesis) affect more than a fifth of the population and manifest in substantial morbidity, mortality, and economic burden. While etiologies range from diabetes and postsurgical complications to neural degeneration and hormonal imbalances, current pharmacological treatments are rarely targeted in mechanism or delivery. Even with surgical intervention, restoration of peristalsis for conditions like achalasia, GERD, and gastroparesis remains inadequate. Conspicuously, in 50% of GERD patients who receive fundoplication surgery following ineffective antisecretory pharmacotherapy, 62% require remedication after a decade. In addition to its role in transporting food, motility also plays a key role in satiety. Following meal ingestion, gustatory secretions, esophageal stretch, and gastric distension elicit vagal afferents to the brainstem nuclei regulating gastric motility, appetite, and satiety in a feed-forward manner. Dysmotile organs lacking such physiological mechano-transduction and afferent signaling alter perceptions of satiety and hunger potentially leading to detrimental behavior.

As an alternative to surgical and pharmacological intervention, electrical stimulation (ES) of the GI tract has been preclinically developed since the 1960s although few approaches have been successfully clinically translated and deployed as approved interventions. One example involves serosal stimulation (Enterra, Medtronic) to alleviate gastroparesis, which is an emerging method, although clinical trial results remain inconclusive and its use remains under the FDA's humanitarian exemption guidance. Several factors contribute to the limited effectiveness of ES. Firstly, while GI peristalsis is a closed-loop process triggered by the temperature and pressure stimuli of food boluses, current ES devices operate in an open-loop fashion, inducing myenteric signaling uncorrelated with food intake and neurochemical physiology. Secondly, peristalsis requires the coordinated activation of circular and longitudinal neuromuscular layers. Single-electrode devices performing point-source ES fail to propagate neuromuscular waves in dysmotile organs. Thirdly, motility disorders implicate both excitatory and inhibitory physiology, while ES can only modulate contractile activity, precluding its utility for hypercontractile pathologies. Fourthly, although a broad range of parameters have been explored to perform GI ES (0.2-50 ms, 3-10 mA, 10-50 Hz, 30 min-24 hours), little consensus or customization to peristalsis has been achieved15,19. Finally, interfacing with the deep-set, interwoven enteric plexi and circular muscle layers necessitates invasive surgery or advanced endoscopy methods. Although natural orifice transluminal endoscopic surgery (NOTES) is an emerging technique for manipulation of submucosal layers, it remains unfamiliar to physicians with few available tools for tissue manipulation or implantation. Consequently, effective treatments for GI motility disorders still await an implantable device that interfaces with the myenteric plexus, withstands cyclic deformation, and allows closed-loop sensing and actuation to reproduce naturalistic neuromuscular activity.

### 1.1 General Device Designs

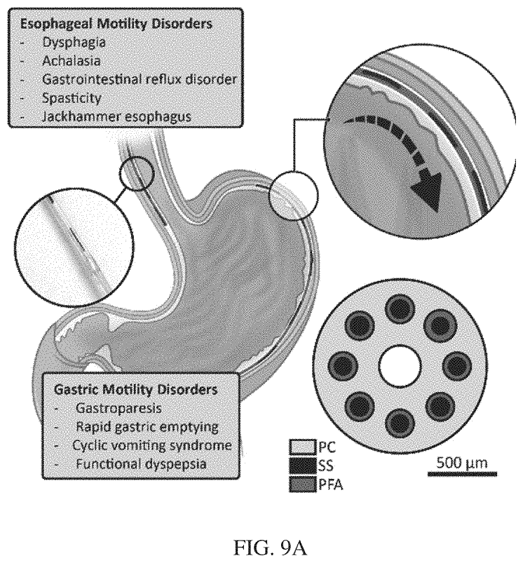

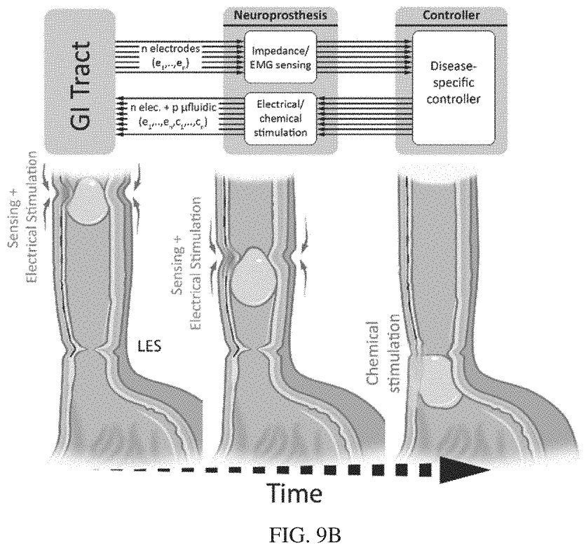

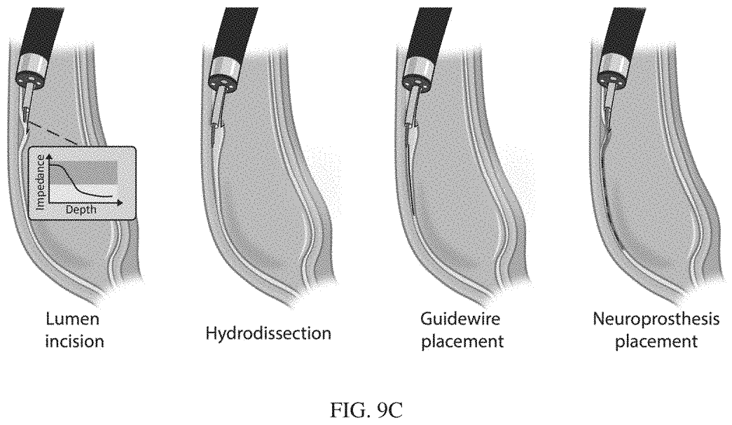

Here, the development and validation of an implantable GI neuroprosthesis which recapitulates ENS control of peristalsis via closed-loop, multichannel, chemical and electrical stimulation in response to sensed food stimuli was described. The neuroprosthesis featured ES contacts spaced at 1 cm intervals longitudinally along the submucosal plane, mirroring the spatial arborization of distal myenteric nerve roots which could jointly innervate smooth and striated muscle layers from their submucosal branch points. The stimulation controller could be customized to mimic physiological vagal efferent signaling to circumvent myenteric pathology, generate coordinated peristalsis, and restore motor function (FIG. 9A). It was hypothesized that the neuroprosthesis would produce effective peristalsis in a paretic esophagus or stomach in response to food stimuli (FIG. 9B). With the capability to mechanically actuate the organ, the neuroprosthesis could also induce artificial afferent mechanosensation. By eliciting stretch sensitive vagal afferents mimicking food ingestion in the fasted state, it was hypothesized that neuroprosthetic stimulation would induce an illusory state of metabolic satiety and/or achieve a metabolic response commensurate with a fed, or satiated state. To overcome challenges associated with implantation, an endoscopic submucosal implantation tool (SIT) that allowed anatomically-precise minimally-invasive implantation of the neuroprosthesis was also developed (FIG. 9C). This tool would allow precise incision of the mucosa, accurate identification, and dissection of the submucosa, and safe implantation of the neuroprosthesis.

FIGS. 9A-9C shows schematics of a closed-loop gastrointestinal neuroprosthesis and minimally invasive submucosal implantation tool. As shown in FIG. 9A, the neuroprosthesis allowed augmentation of esophageal and gastric motility through multichannel ES and chemical stimulation. Insets demonstrate patterned stimulation that mimics physiological signaling and dimensions of the implant. Upon sensing a bolus of food, programmed patterns of ES and/or chemical stimulation would contract and inhibit the neuromusculature to recreate peristalsis (FIG. 9B). The SIT would then facilitate minimally invasive and precise implantation through the following steps (FIG. 9C): 1) an overtube was passed through an endoscopic channel, and while a self-expanding nitinol hook pulled counter tension on the luminal wall, a penetrating needle incised the lumen and advanced until the submucosa was localized based on tissue impedance; 2) hydrodissection would then separate the lumen from the muscularis; 3) a guidewire was then advanced to dissect the plane along the curvature of the organ; and 4) the neuroprosthesis was then inserted into the submucosal space.

### 2. Design, Fabrication, and Validation of Submucosal Implantation Tool

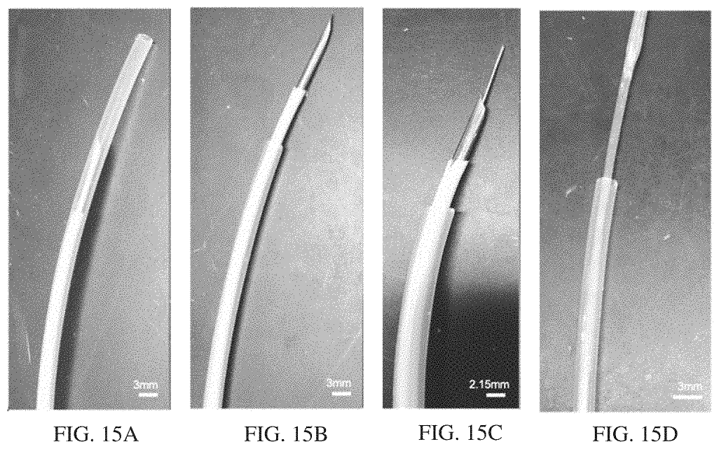

A minimally invasive submucosal implantation tool was designed with the considerations of endoscopic incision, anatomic localization, dissection, surgical risk mitigation, and precision control availed at a length of two meters through the 3.8 mm working channel of standard endoscopes. Key design elements and detailed procedural description are described below and shown in FIG. 9C and FIGS. 15A-15D.

2.1. Implantation Procedure with Submucosal Implantation Tool

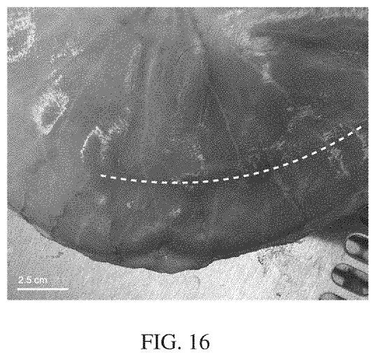

As shown in FIGS. 15A-15D, the submucosal implant tool comprises the following components: an overtube (2.8 mm OD) (FIG. 15A), an incision needle (2.15 mm OD) (FIG. 15B), a localization guide wire (FIG. 15C), and a prosthesis through the overtube (FIG. 15D). As shown in FIG. 9C, for use, the pre-loaded 1.9 cm diameter overtube was inserted through the endoscopic channel and advanced to the proximal site of implant. The incision catheter was advanced while holding the overtube in place relative to the endoscope, facilitated by an o-ring stabilizer. A nitinol hook was deployed in the adjacent channel to hold tension on the site of incision. The incision catheter was advanced while continually monitoring impedance to detect localization in the submucosal layer (<100 kOhms, FIG. 16). Hydrodissection was then performed with 10-35 mL saline while monitoring the expansion of the layer intraluminally via endoscopic video. The guide wire was then advanced to a length commensurate with the prosthesis to ensure clear separation of the fascial layer in the path into which the prosthesis would glide. The guide wire was removed and the prosthesis was inserted. Fibrin glue could be infused through the microfluidic channel or a Carr Locke needle to secure the end of the prosthesis in the tissue layer. Leads could then be tunneled out to a subcutaneous pocket or through a PEG tube to a stimulator.

### 2.2 Rationale for Design of Incision Needle

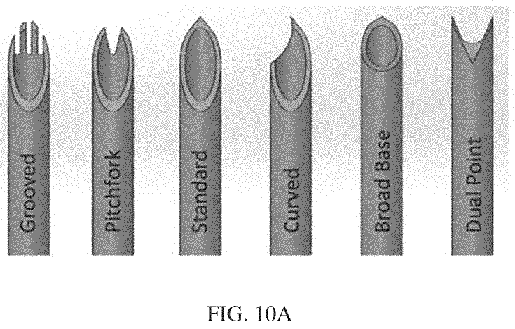

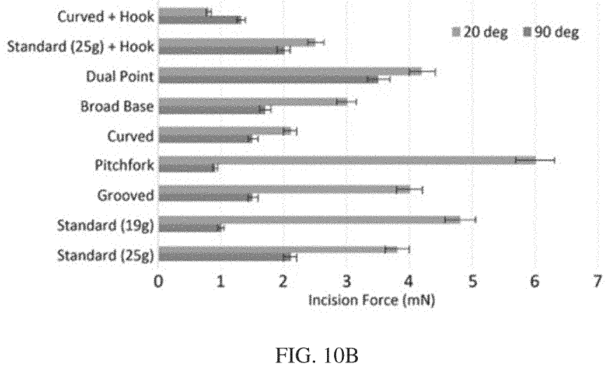

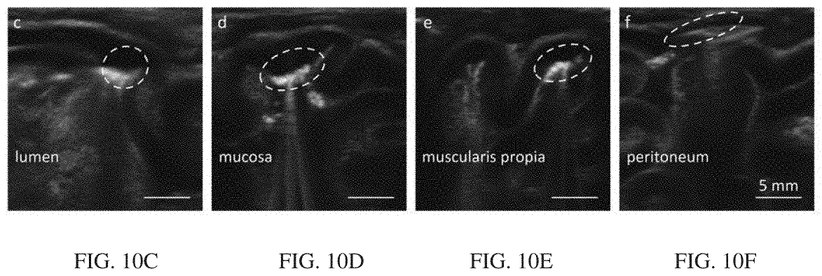

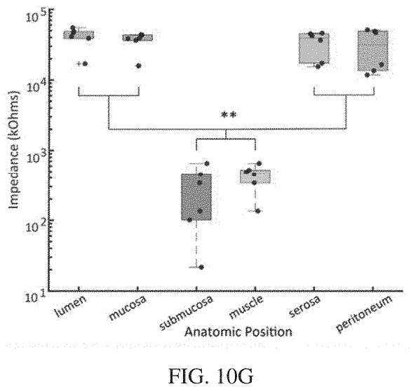

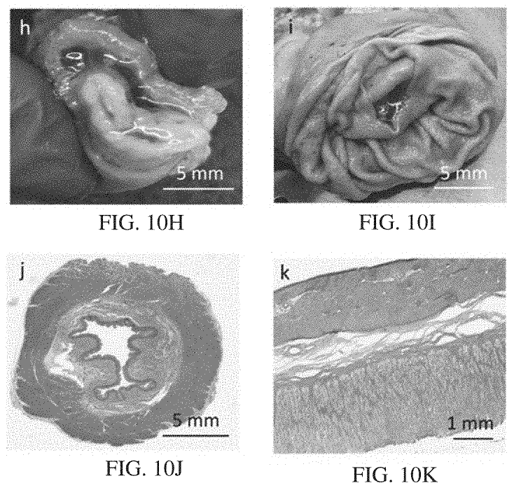

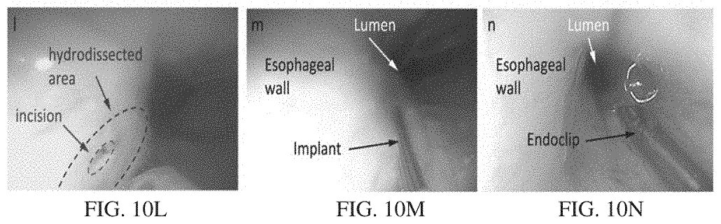

Given the shallow angle of attack for endoscopic incision, a needle profile capable of low-force incision of the distensible mucosa without slipping was necessary to prevent trauma or perforation. Needles having the following geometries were designed and tested: triple grooved tip, pitchfork shape, standard bevel (25 g and 19 g), curved tip, broad base, and dual point (FIG. 10A) with and without a self-expanding nitinol hook which applies a stabilizing counter force.

A variety of designs were created to optimize the needle edge for penetration of the esophagus at low force without high perforation risk. The angling of an endoscope created, at best, a 20-degree angle between the catheter and the tissue. Thus, the following designs were test: i) the triple grooved tip and pitchfork tip were developed with the aim of catching the tissue that glides within the grooves, creating a wedge scissoring effect; ii) the curved tip was developed to implement the slide-push effect during penetration, which was known to cause a reduction in cutting forces and displacement of the medium; iii) the broad base needle tip was designed with a reduced bevel angle to increase the angle of attack of the cutting surface on the tissue, reducing the chance of slip during the incision; and iv) the dual point design was intended to allow for a cutting edge to be in contact with the tissue regardless of twist angle, as it was difficult to adjust the twist of the tip once in the endoscope. These were all compared against standard bevel designs to evaluate their force requirement upon penetration of the compliant GI tissues.

FIGS. 10A-10N illustrates the safety and efficacy testing of SIT. As shown, needle designs were tested to optimize penetration force (FIG. 10A). Force of penetration was significantly increased at 20 degrees as compared to a 90° of attack (FIG. 10B). The curved design with a hook demonstrated the lowest penetrating force. Impedances were recorded as the needle advanced through the lumen, mucosa, muscularis propia, and peritoneum (FIGS. 10C-10F). The impedance of the submucosa and muscularis were significantly different from other layers, which allowed precise localization (**p<0.01, student's t-test) (FIG. 10G). Injection of saline allowed accurate dissection of the submucosal layer as seen in gross images and histology of the esophagus (FIGS. 10H and 10J) and stomach (FIGS. 10I and 10K). Endoscopic visualization aided the incision (FIG. 10L), implantation (FIG. 10M), and closure with one resolution clip (FIG. 10N).

Penetration force (1 cm/minute linear displacement) at 20° and 90° of attack on porcine stomach and esophageal tissue demonstrated significantly higher forces at the 20° of attack (FIG. 10B). The curved design with the nitinol hook provided the greatest reduction in force as compared to standard 19- and 26-gauge needles (p<0.05, student's t-test, n=5 trials) and was selected for implementation in the submucosal implantation tool. In vivo, the tool's incision in the esophagus and stomach measured 1.8±0.3 mm and 2.12±0.12 mm (n=3 each), respectively. No stretching, sliding or tearing of tissue were observed.

While visualizing the stomach using ultrasound laparoscopically, the tool's needle was endoscopically introduced into the lumen and sequentially advanced through each layer to the peritoneum, while monitoring impedance and position (FIGS. 10C-10F). It was observed that the electrode impedance could be used to distinguish the submucosa and muscularis propria from the mucosa and serosa. The SIT probe measured impedances in the submucosa and muscle layers two orders of magnitude lower than the lumen, mucosa, serosa, and peritoneum (FIG. 10G, n=6 per anatomic location, p<0.01, student's two-tailed heteroscedastic t-test), thus allowing localization of the submucosal layer.

The SIT accurately localized and allowed dissection of the submucosal plane following the natural curvature of the esophagus and stomach ex vivo (n=7) and in vivo (n=4) without perforation, hematoma, blood loss or gross tissue trauma (FIGS. 10H-10I and FIG. 16). Histological tissue analyses corroborated accurate identification of the desired plane and effective separation of muscularis planes and revealed no unanticipated tissue damage (FIGS. 10J-10K). Methylene blue and histological stains were identified exclusively in the targeted locations. The main endoscopic stages of 1) localization & dissection, 2) implantation and 3) closure are shown in FIGS. 10L-10N.

### 3. Design and Fabrication of GI Neuroprosthesis

The design of the neuroprosthesis was optimized for endoscopic delivery, efficacious neuromuscular modulation, electrical and chemical stimulation, structural flexibility, and biocompatibility with submucosal tissues. The thin (1.250±0.1 mm diameter) and flexible device form factor was chosen to be compatible with standard working channels of endoscopes (2.8-3.2 mm), withstand endoscopic implantation, habituate along a tissue plane, and move with the organ (FIG. 9A). The neuromuscular signaling patterns that gave rise to peristaltic activity ex vivo to recapitulate them in the prosthesis was obtained using the method outlined below (See, e.g., Section 3.1 Ex vivo characterization of peristalsis); and sequential activation of submucosal myenteric branches elicited peristalsis of the arborized muscle segments in both circular and longitudinal muscle layers, which synergize for peristaltic motion.

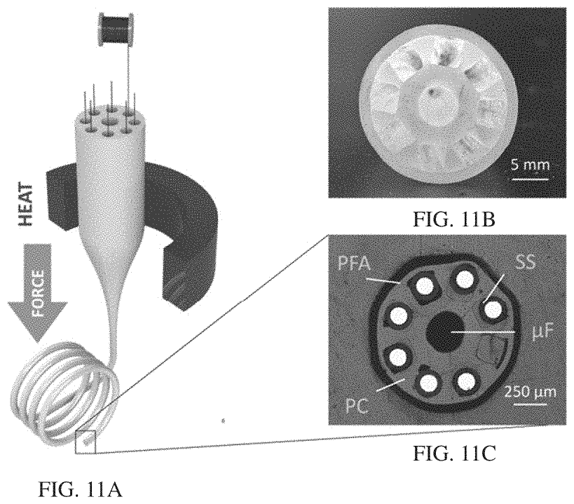

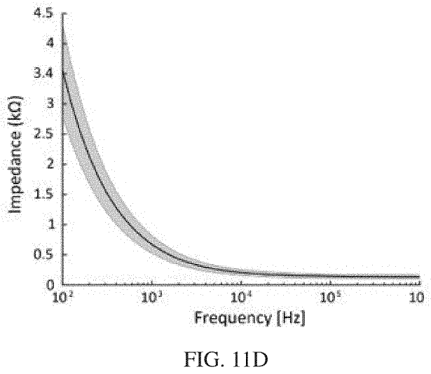

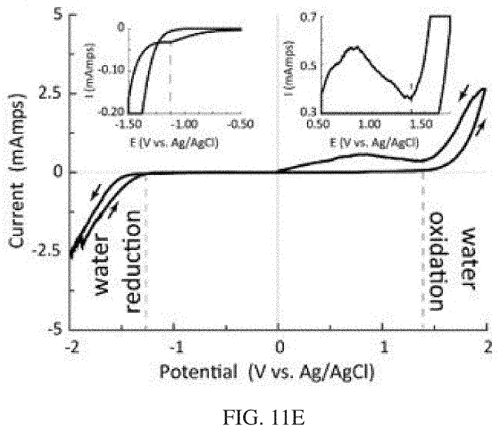

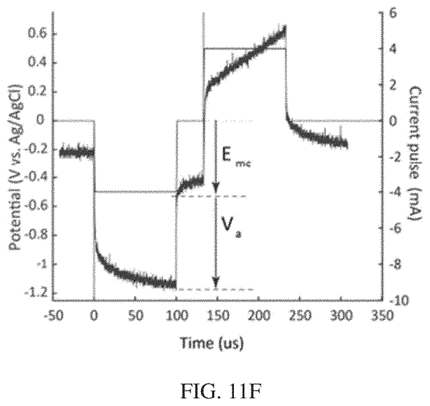

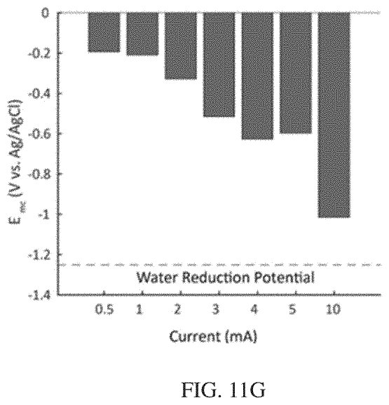

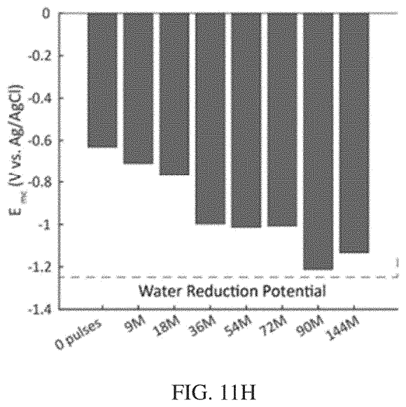

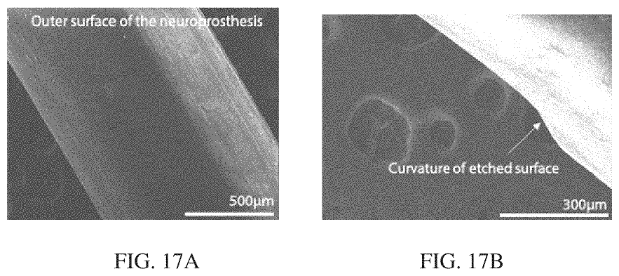

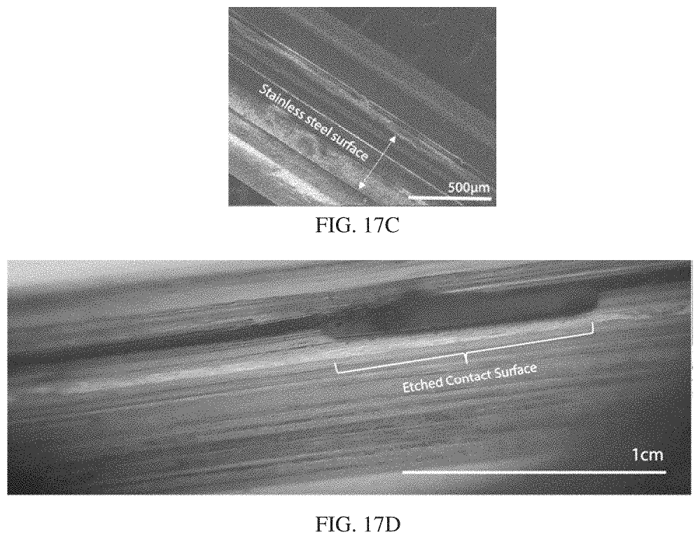

Stainless steel was chosen for its durable, inert properties to serve as the electrode material, with the device clad in polycarbonate, selected for its properties of flexibility and biocompatibility. A central microfluidic channel allowed the elution of neurochemicals for myenteric modulation. To create a multimodal neuroprosthesis, a fiber drawing process was recently adapted for creating multifunctional probes for neural recording and stimulation. During fiber drawing the macroscale template of the neuroprosthesis was transformed into the mm-scale fiber through application of controlled heat and stress (FIGS. 11A-11C). To integrate high-melting temperature (Tm) stainless steel electrodes into the polycarbonate cladding with significantly lower glass transition point, a material convergence technique was employed. The electrodes were then exposed to the surface via laser etching of the polymer cladding, and the electrochemical surface area at the electrode-tissue interface was optimized to maximize current injection and decrease impedance (See Section 10. Methods, and FIGS. 17A-17D).

FIGS. 11A-11C illustrates fabrication and characterization of the closed-loop gastrointestinal neuroprosthesis. A schematic of the thermal drawing process during which a macroscopic preform (FIG. 11B) was heated and stretched into mm-scale fiber (FIG. 11C). During this process, stainless steel microelectrodes were fed into the preform and embedded into the final fibers. Electrochemical impedance spectrum of the stainless-steel electrodes exposed through the polymer cladding using laser etching (FIG. 11D) and cyclic voltammogram of the stainless-steel electrodes in 1× PBS (FIG. 11E) were obtained. Representative potential transient response to a +−4 mA symmetric, biphasic current pulse of 100 μsec half-phase period and a 33.3 μsec interphase delay, and estimation of the maximum cathodic potential Emc was obtained (FIG. 11F). Emc of the electrode following a 0.5, 1, 2, 3, 4, 5, and 10 mA current pulse (FIG. 11G) and accelerated aging of the electrode: Emc of the electrode following 9, 18, 36, 54, 72, 90, and 144M continuous 4 mA current pulse (FIG. 11H) were acquired.

### 3.1 Ex Vivo Characterization of Peristalsis

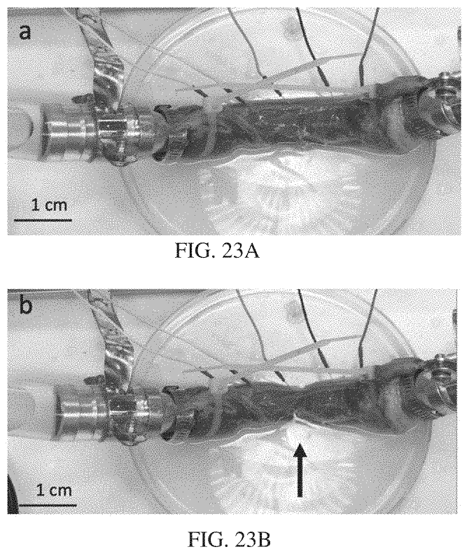

Ex vivo, the dynamics of peristalsis were studied using the tissue maintenance system. Electrodes were inserted into serosal, submucosal, or mucosal layer of the esophagus and the EMG response was captured in response to 40 Hz, 3 ma, 3 s stimulation. The amplitude and strength of contraction were found to be strongest when electrodes were placed in the submucosal layer. Spacing of the electrodes was also varied between 0.5 and 2 cm in 0.5 cm increments and the spatial dynamics of contraction were studied, yielding an optimal spacing of 1 cm between contacts (FIGS. 23A-23B). In order to propagate a bolus using ES pulses, the proximal segment needed to remain contracted for at least half the duration of the pulse to propagate in the anterograde direction. These insights were used to design the neuroprosthesis and controller.

### 4. Electrochemical Characterization of Stimulating Electrodes

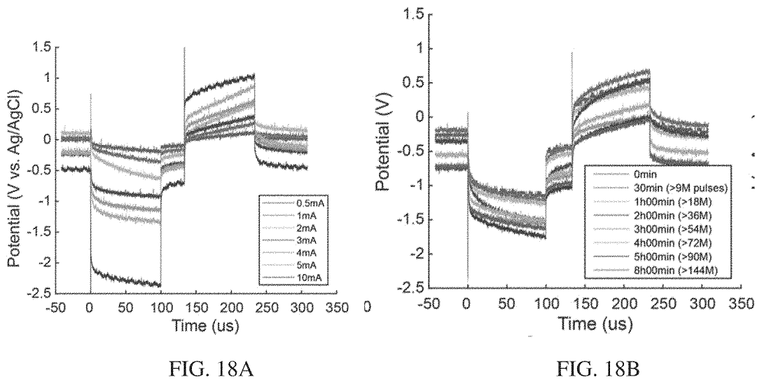

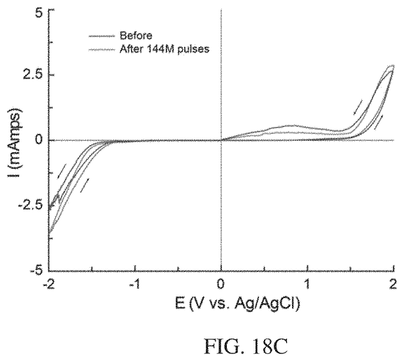

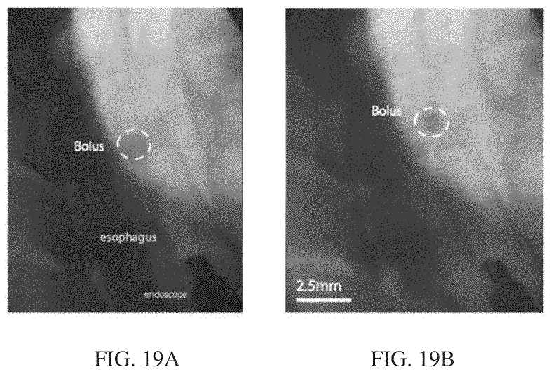

To characterize the electrical and electrochemical properties of the stimulating electrodes exposed by laser etching, electrical impedance spectroscopy was performed. The impedance values were recorded at 100 Hz, 1kHz and 10 kHz were 3.56±0.78 kΩ, 0.67±0.15 kΩ, 0.21±0.05 kΩ, respectively (FIG. 11D). Using cyclic voltammetry, the water-stability window was estimated between potentials of +1.37 and −1.25 V vs. Ag/AgCl, which marked the onsets of oxygen evolution reaction (OER) and hydrogen evolution reaction (HER), respectively (FIG. 11D). Charge injection capacity of the electrodes was then determined on the basis of voltage transients in response to biphasic, symmetric rectangular current pulses (half-phase period=100 μsec, interphase delay=33.3 μsec) ranging in amplitude between 0.5-10 mA (FIGS. 11F-11G). Since the onset of HER (1e-transfer reaction) was found to be lower than that for OER (4e-transfer reaction), the former potential limit was used to calculate the CIC values. It was observed that the maximum cathodic potential (Emc) of stainless-steel electrodes was below the water reduction potential of −1.25 V for current amplitude ≤10 mA (FIG. 11G, and FIG. 18A). Additionally, accelerated aging tests of the neuroprosthesis were conducted in PBS by applying 144×106 biphasic pulses with 4 mA, the target stimulation amplitude of 2 mA with a safety factor of two. The maximum cathodic potential (Emc) measured after 9M, 18M, 36M, 54M, 72M, 90M, and 144M pulses remained below the water reduction potential of −1.25 V indicating long-term stability of the electrodes (FIG. 11H and FIG. 18B). Note that 144×106 pulses corresponded to ˜11 years of use, assuming 900 swallowing events per day, each assisted by the neuroprosthetic stimulation. Finally, a cyclic voltammogram collected following the accelerated aging of the electrodes confirmed the stability of the electrodes at 4 mA current amplitude (FIG. 18C).

To evaluate the impact of repeated mechanical deformation during peristalsis on the electrodes, the electrical impedance and E_{mc} (2 mA current) was measured following 1, 10, 100, 1000, and 10000 buckling cycle (5 mm displacement, 1 cm/sec). The impedance value varied between 2-4 kΩ on average, while the E_{mc} remained below the water reduction potential of −1.25V, demonstrating that the electrodes weren't substantially affected by the repeated mechanical stimulation. Finally, the mechanical properties of the neuroprosthesis was characterized by quantifying its bending stiffness in a single cantilever mode. It was found that neuroprosthesis exhibited bending stiffness values of 516-645 N/m over the frequency range of mammalian locomotion, respiration, and heartbeat. Stress-strain testing of the neuroprosthesis further showed that the tensile behavior of the neuroprosthesis in the elastic regime was dominated by the properties of polycarbonate with a Young's modulus E=10.1±0.66 GPa (n=3) (EPolycarbonate=2.2 GPa, ESteel=200 GPa), while the neuroprosthesis exhibited a flexural modulus E_f=400.0±15.0 MPa (n=3) during three-point bending testing which was well below the flexural modulus of a solid polycarbonate beam (2.3 GPa).

### 5. Design of Closed-Loop Controller and Optimization of Stimulation Parameters

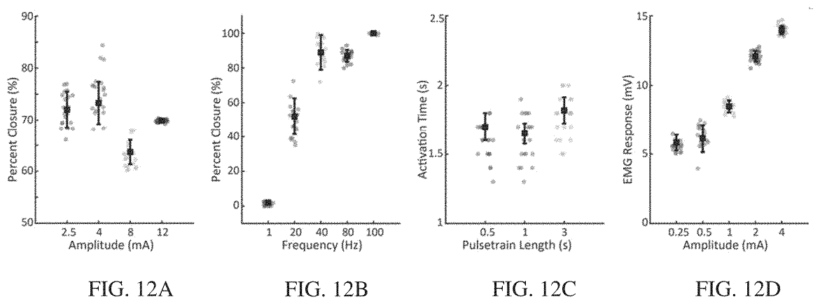

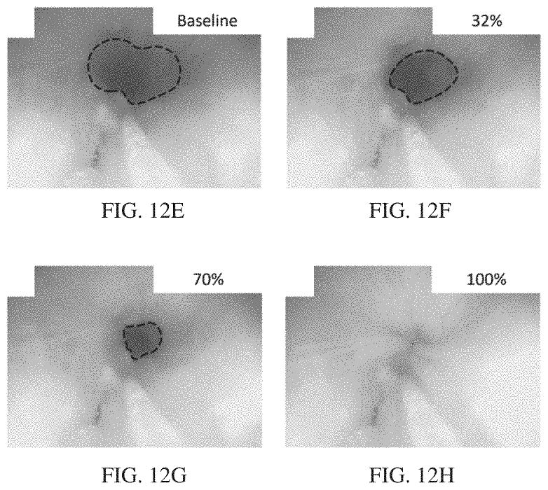

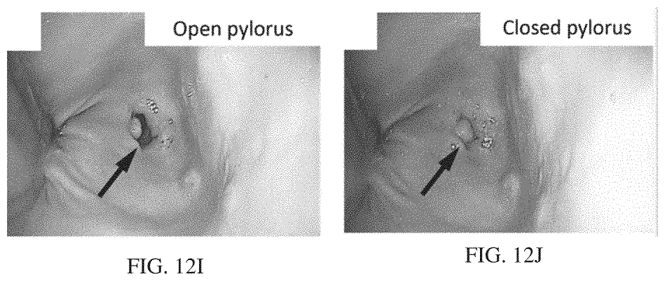

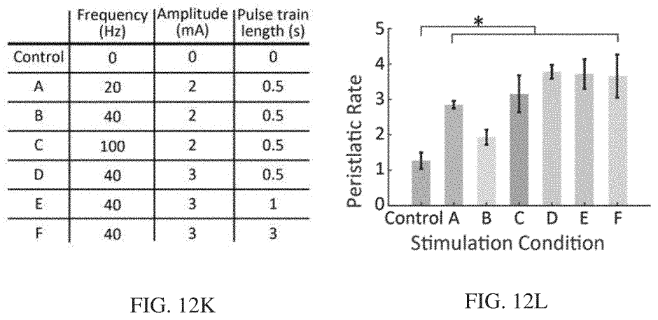

To mimic natural peristalsis, a closed-loop controller capable of executing a coordinated ES pattern to induce peristalsis upon sensing a bolus of food was designed. The controller's stimulation pattern and parameters (Table 1) could be customized to address disease manifestation in a patient-specific manner. As a proof of concept here, The controller was designed for functional esophageal paresis and depressed gastric motility using a model of induced hypomotility (See, e.g., Section 10. Methods). ES stimulation parameters were optimized through a comparative characterization of peristaltic dynamics using in vivo and ex vivo models using commercial electrodes. For the esophagus, a matrix of stimulation parameters was tested (frequency: 20, 40, 100 Hz|peak-to-peak amplitude: 1, 2, 3, 4, 6, 8 mA|pulse train lengths: 0.5, 1, 3 s|n=4 animals|10 repetitions of each condition, FIGS. 12A-12D). Neural activation was expected at pulse train lengths of 0.5 s and 1 s, while 3 s was expected to activate muscle directly. In the case of the esophageal controller, repeated boluses within 5 s triggered a temporary pause in the actuation commands to mimic natural deglutitive inhibition. Image analysis was performed to calculate the percentage of closure in the esophageal lumen (FIGS. 12E-12H). At a 40 Hz 1 s pulse trains, 4 mA yielded complete esophageal closure, with higher amplitudes leading diminishing returns due to muscle fatigue or hypertonicity (FIG. 12A). Utilizing a 4 mA amplitude and 0.5 s pulse train, a frequency sweep revealed that 40 Hz stimulation produced sustained esophageal contraction with 89±4% closure (FIGS. 12B, 12H). At 4 mA and 40 Hz, a sweep of pulse train lengths revealed no significant difference between conditions (FIG. 12C, p>0.05, student's t-test). Thus, 40 Hz, 4 mA and 0.5 s pulse duration, which allowed full esophageal closure, were selected for the closed-loop controller for esophageal hypomotility (FIG. 12H, Table 1). EMG graded monotonically with stimulation amplitude (FIG. 12D) between 0.25 and 4.0 mA, availing a linear stimulation-response relationship for controller optimization. Ex vivo point source stimulation activated neuromusculature 7.2±4.5 mm proximally and distally (n=35 trials) motivating 10 mm electrode spacing on the neuroprosthesis. Electrically-stimulated segments relaxed (full opening) 1.7±0.3 s following the end of the pulse trains. Thus, esophageal ES was programmed to repeat at 1 s intervals consistent with normal swallowing dynamics characterized by temporal overlap in the distal and proximal segment contraction mitigating reverse propagation.

TABLE 1
Closed-loop program parameters for neuroprosthesis.

| | Optimized Value |
|---|---|
| | for Hypomotility Model |
| Amplitude (peak to peak) | 4 mA |
| Frequency | 40 Hz |
| PulseWidth | 100 μs |
| Pulse_Train_Length | 0.5 s |
| Order | 1,2, 3,4, 5, 6,7 |
| Time_Between_Pulses | 1 s |
| Impedance_Frequency | 200 kHz |
| Baseline change for bolus | 10% |
| detection | |
| Refractory period | 10 s |
| Deglutitive inhibition | 5 s |

Using an optimization strategy analogous to that for esophageal ES, parameters for gastric motility were optimized through a comparison of the rate of pyloric closure (FIGS. 12I-12J), which was proportional to peristaltic activity in the fasted state in response to six sets of parameters (FIG. 12K). FIG. 12L demonstrates the average and standard deviation for at least 7 minutes of stimulation under each condition (with 10 minutes of rest in between each) for each animal (n=6). All ES conditions, excluding set B, produced a significant increase in peristaltic rate as compared to the baseline control (p<0.01, student's t-test). Stimulation settings in A were chosen for implementation in the neuroprosthesis as these provided a significant increase with the most minimal intervention.

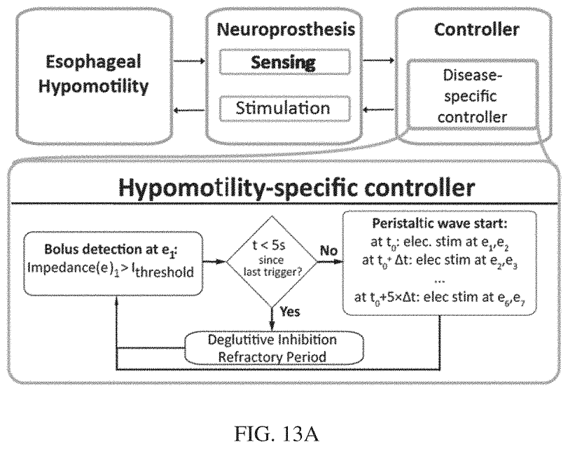

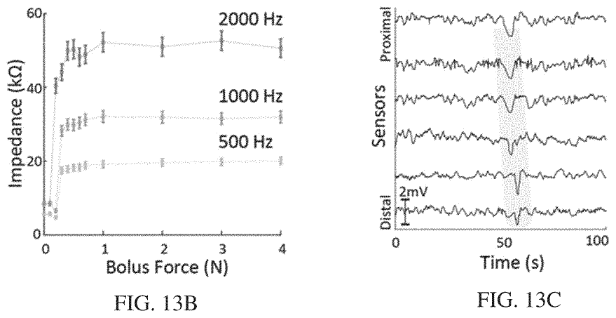

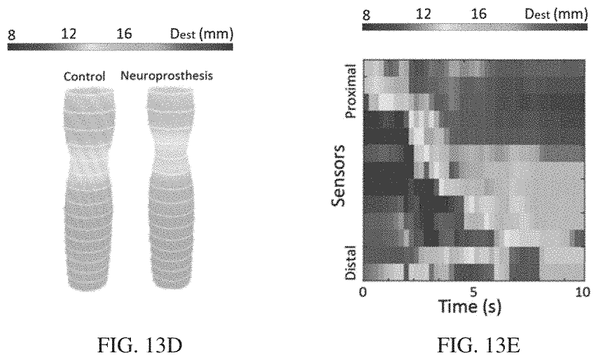

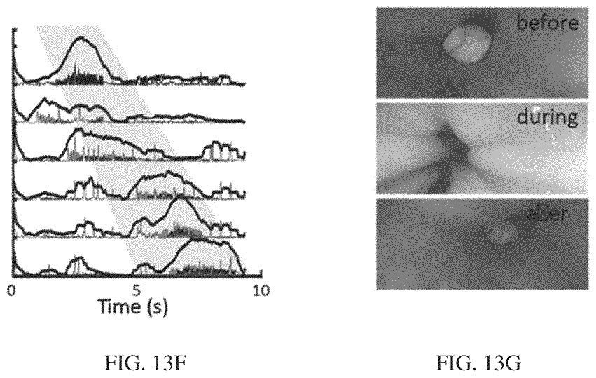

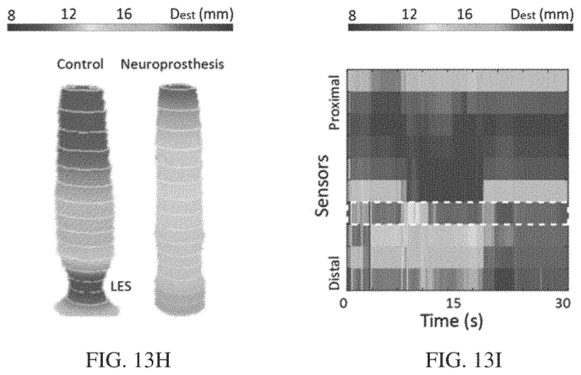

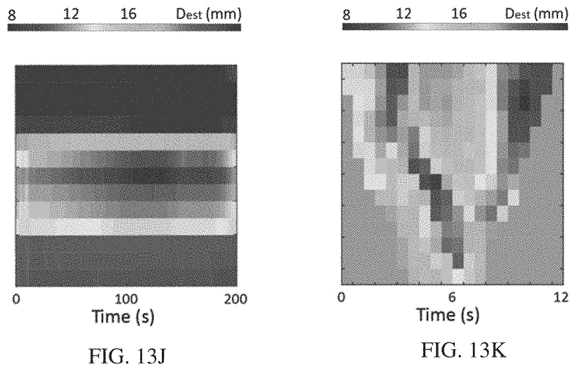

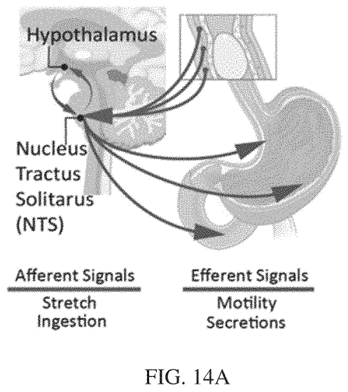

The detection of food stimuli, the feedback component of the closed-loop controller, was performed by the proximal electrodes using differential amplification of the impedance measured at 0.5, 1, and 2 kHz frequencies (FIGS. 13A-13B). A significant increase in the impedance was observed at forces greater than 0.2N, which corresponded to the pressures exerted by semi-solid and solid foods. EMG detected on each of the electrodes could also be used to detect ingestion or initiation of peristalsis (FIG. 13C).

### 6. Neuroprosthetic Augmentation of Motility

The neuroprosthesis was implanted in the esophagus of a swine model of depressed motility (n=3) and its functionality was assessed via endoscopy, fluoroscopy, and intraluminal manometry. No significant differences were found between pre- and post-implantation distensibility index, minimum cross-sectional area (CSAmin), or pressure at baseline (8.2-9.4 mmHg at 20 mL inflation), indicating that the neuroprosthesis itself did not adversely affect the baseline characteristics of the esophagus (p<0.05, n=6 trials, student's t-test). The neuroprosthesis effectively generated swallowing movements with duration and pressure of axial contractions not significantly different from those of natural swallow reflexes in control conditions (p<0.05, student's t-test, n=30 trials FIG. 13D). Inflation of the intraluminal manometer to 20 mL manifested in forces and the corresponding changes in electrode impedance above the sensing threshold of the neuroprosthesis, triggering sequential stimulation yielding a peristaltic wave as measured by manometry and EMG (FIGS. 13E-13F). Peristaltic waves created by the neuroprosthesis were insignificantly different from reflexive swallows in sequence and pressure and fell within physiological bounds. To test the efficacy of the neuroprosthesis in propelling food, radio-opaque boluses were placed in the proximal esophagus, triggering activation along the neuroprosthesis which propelled the bolus down the esophagus (FIG. 13G, and FIGS. 19A-19B). These results demonstrated that the neuroprosthesis could be used to induce peristalsis in hypomotile conditions and effectively propel food in a paretic esophagus.

FIGS. 13A-13K illustrates neuroprosthetic generation of peristalsis. FIG. 13A shows a controller scheme of the closed-loop actuation of the GI tract using the neuroprosthesis. The controller parameters are calibrated on a given disease. Impedance amplitude at 500 Hz (green), 1000 Hz (blue), and 2000 Hz (purple) were acquired (FIG. 13B) and demonstrated a significant difference between baseline and the ingestion of a bolus applying forces greater than 0.2N. EMG of the stomach upon stimulation by the neuroprosthesis was acquired and demonstrated slow wave propagation recorded along 6 consecutive electrodes (FIG. 13C). Representative intraluminal manometry measurement of the esophagus during a reflex-initiated swallow and neuroprosthesis-actuated contraction were acquired and demonstrated similar characteristics in contraction strength and propagation (FIG. 13D). As shown in FIG. 13E, neuroprosthesis-actuation peristalsis in the esophagus demonstrated a coordinated swallowing pattern, including relaxation of the LES in response to microfluidic glucagon infusion at the distal end of the manometer. Filtered EMG and raw signal were recorded at each electrode during neuroprosthesis-initiated peristalsis in the esophagus (FIG. 13F). FIG. 13G shows endoscopic visualization of the bolus before (top), during (middle) and after (bottom) contraction of the esophageal muscle induced bolus propagation. Representative intraluminal manometry of the esophagus at rest demonstrating a tonic LES was acquired (FIG. 13H). As shown, glucagon infusion by the neuroprosthesis yielded relaxation of this LES (FIG. 13H, right). As shown in FIG. 13I, temporary relaxation of the LES induced by glucagon infusion started at t=0. As shown in FIG. 13J, continuous stimulation yielded less than 20% decrease in force production over a 200 second trial. Representative manometry during antero- and retrograde peristalsis is shown in FIG. 13K.

In addition to demonstrating peristalsis in a model of hypomotility, the neuroprosthesis' microfluidic channel was leveraged to release inhibitory neurotransmitters facilitating relaxation of hypertonic musculature for conditions such as achalasia and spastic esophagus. The neuroprosthesis' distal tip was implanted in the lower esophageal sphincter and glucagon was infused through the microfluidic channel. This chemical stimulation yielded relaxation of the sphincter within 10-20 s. Average luminal distensibility substantially increased from 6.8±1.2 mmHg to 15.8±1.7 mmHg (FIGS. 13H-13I). Muscle tone returned within 1.5 minutes whereupon repeated infusions led to relaxation. Injection of saline produced no measurable effect and served as negative a control.

To further validate the long-term efficacy of the neuroprosthesis, the system was investigated further to gauge its capability in eliciting repeated actuation without inducing muscle fatigue, a known adverse effect of electrical simulation due to altered recruitment dynamics. Continuous stimulation was performed for 160 seconds, which was substantially longer than relevant therapeutic applications, and less than 10% reduction in force production was observed, suggesting minimal fatigue induction (FIG. 13J). This may be due to the selecting of lowest effective stimulation parameters (amplitude, frequency, and duration) to achieve peristalsis, thus preventing unnecessary over-excitation and muscle fatigue. The neuroprosthesis could be programmed to tailor the actuation pattern to each patient's pathology and physiology by adjusting the timing, spacing, refractory period, strength of contraction, and sequence of activation. As a proof of concept, the neuroprosthesis was programmed to elicit anterograde and retrograde peristaltic waves (FIG. 13K).

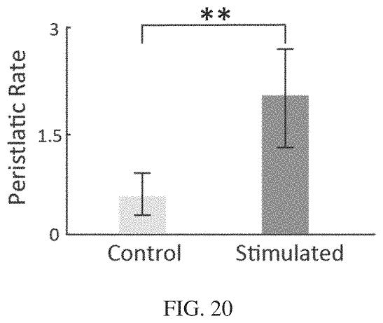

Finally, to assess its influence on gastric motility, the neuroprosthesis was implanted in the corpus of the stomach using the endoscopic submucosal implantation tool (i.e., SIT) described previously. Under deep anesthesia when no peristaltic waves were observed and the pyloric closure rate had dropped below once per min, 0.5 s pulse trains of 20 Hz stimulation spaced 3 s apart on consecutive electrode were applied. A two-fold increase in the peristaltic rate was observed over a 20-minute period (FIG. 20, p<0.05, student's t-test, n=4). In all trials, the neuroprosthesis complied with the tissue movement, exhibiting no sliding, protrusion, or impediment to normal peristalsis. These results suggest that the neuroprosthesis can be used in a chronic setting to artificially generate peristalsis and increase the motility rate.

### 7. Biocompatibility

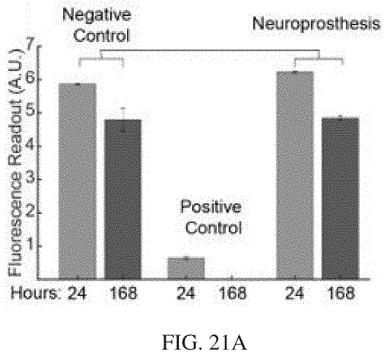

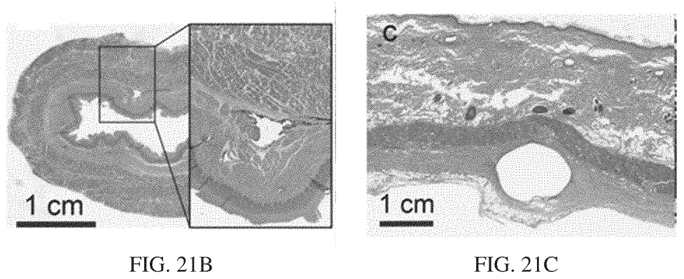

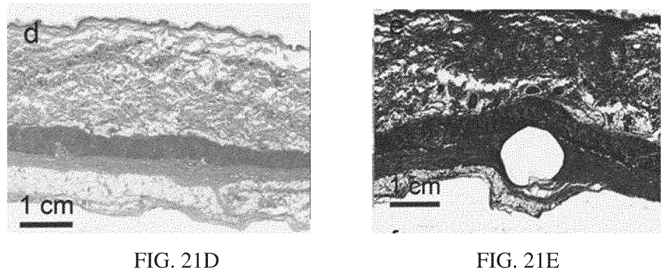

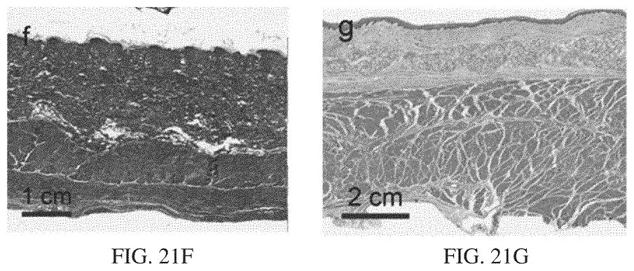

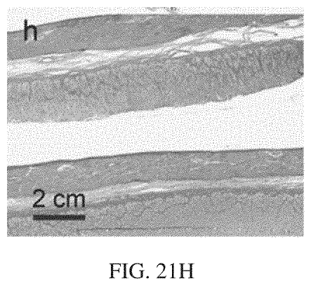

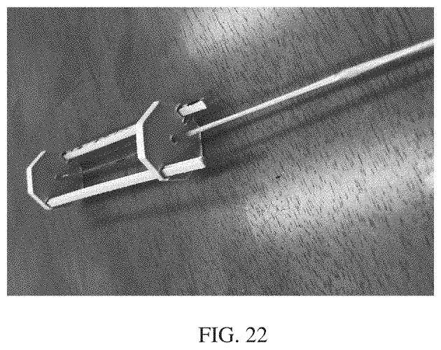

To evaluate the chronic tissue-material interactions of the neuroprosthesis containing polycarbonate and stainless steel, in vitro and in vivo biocompatibility testing were performed (See, e.g., Section 10. Methods). In vitro, an extract exposure test on C2C12 cells demonstrated no significant toxicity at 24 or 168 hours as compared to a negative control (p<0.05, student's t-test). In vivo, 7 days following implantation, esophageal distensibility and dynamics demonstrated negligible differences to pre-implantation measurements. Complete healing of tissues and no substantial foreign body response or migration was observed in stomach and esophageal tissues (See, e.g., Section 6.1 Assessment of biocompatibility; FIGS. 21A-21H).

### 7.1 Assessment of Biocompatibility

An extract exposure test on C2C12 cells demonstrated no significant difference in toxicity between the negative control and the neuroprosthesis material at 24 and 168 hours (FIG. 21A), while 40 um of toxic MG-132 treatment, used as positive control, resulted in significantly decreased cell viability (p<0.05, student's t-test). Following seven days of implantation in a swine esophagus, intraluminal manometry was performed to elicit the swallowing reflex, which occurred naturally at inflations greater than 20 mL. Compared to the baseline prior to implantation, minimal differences were observed in the distensibility and CSAmin of the esophagus, indicating the negligible impact of the prosthesis on organ dynamics (Table 2). Further, no significant foreign body reactions, scarring or migration were observed (FIG. 21B). Finally, accurate placement of the prosthesis in the submucosal layer, adjoining the circular muscle was confirmed (FIG. 21B, inset). Hemotoxylin and eosin staining of subcutaneous tissues implanted with the neuroprosthesis for 14 and 28 days demonstrated a thin collagenous fibrous capsule forming around the material (FIG. 21E), but no substantial foreign body response (FIG. 21C) as compared to non-implanted control sections (FIG. 21D-21F). During endoscopy and following explanation of the neuroprosthesis, the esophageal mucosa and lamina propia at the incision site demonstrated full healing with little fibrosis (FIG. 21G). Submucosal collagen was found to be slightly separated and cushioning the implant as compared to a control segment of the stomach, although no adverse biocompatibility concerns were seen (FIG. 21H).

TABLE 2
Esophageal manometery parameters before and
after implantation of the neuroprosthesis

| | Before | | 7 days after | | Percentage | |
|---|---|---|---|---|---|---|
| Inflation of | implantation | | implantation | | Change (%) | |
| Manometer | Disten- | | Disten- | | Disten- | |
| (mL) | sibility | CSAₘᵢₙ | sibility | CSAₘᵢₙ | sibility | CSAₘᵢₙ |
| 20 | 4.7 | 17 | 4.6 | 17 | 0 | 0 |
| 30 | 4.7 | 18 | 4.6 | 17 | 0 | 5.0 |
| 40 | 4.7 | 18 | 4.9 | 21 | 4.0 | 14 |

### 8. Metabolic Neuromodulation

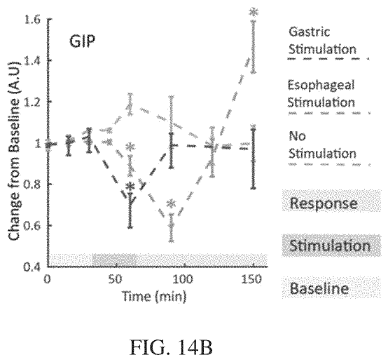

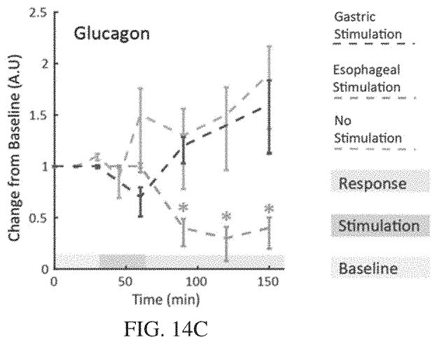

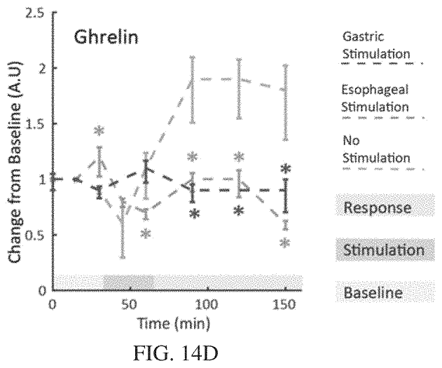

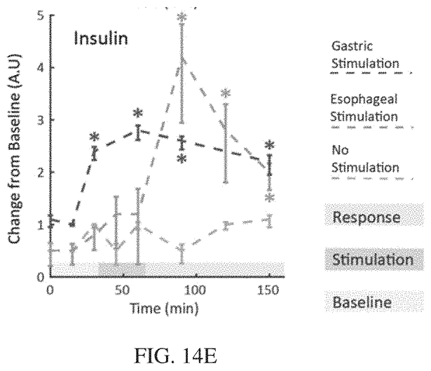

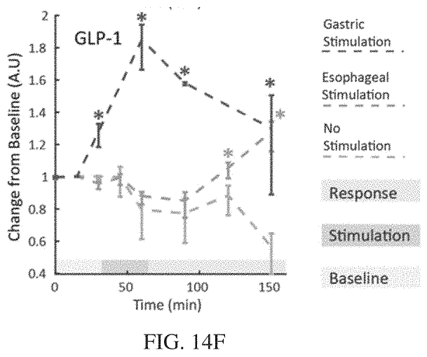

When implanted in the esophagus, the neuroprosthesis resided alongside primary sensory and low-threshold mechanoreceptors innervating the thoracic esophagus which originated from the nucleus tractus solitarius (NTS) and dorsal motor nucleus (DMN) in the brainstem. In the stomach, antral placement was performed to maximize contact with stretch receptors which directed afferents to the NTS. The NTS and DMN then relayed sensed information to the hypothalamic neurons which integrated ingestion, hunger, and satiety signals, in turn, efferently controlling hormonal secretion and motility (FIG. 14A). Satiety was modulated by generating vagal afferents artificially signaling ingestion and gastric motility. Mimicking peristalsis following ingestion, 30 min of sequential stimulation (esophagus: 40 Hz, 2.5 mA, and 0.5 s; stomach: 20 Hz, 2 mA, 0.5 s) was performed with 30 s rest periods separating 120 s stimulation epochs to mitigate fatigue. Satiety and disorders thereof are profiled by measuring levels of the metabolic hormones, such as GLP-1, insulin, glucagon, GIP and ghrelin, which generally rise and fall with hunger and a fed state. Therefore, to characterize the effect of the neuroprosthesis on the body's satiety response, metabolic hormone levels in the blood were measured at 0, 15, 30, 45, 60, 90, 150 minutes, with stimulation occurring between the 30-60th minute marks. Esophageal actuation in the fasted state resulted in a significant increase in glucagon-like peptide-1 (GLP-1) and insulin along with a moderate suppression of ghrelin and no change in glucagon in the response phase compared to the baseline—commensurate with a fed state (p<0.05, student's t-test, FIGS. 14B-14F). Gastric inhibitory polypeptide (GIP) initially decreased, but then increased beyond control levels. Gastric actuation resulted in a significant increase in GLP-1, insulin, and glucagon secretion (p<0.05, student's t-test) compared to baseline, while ghrelin and GIP levels remained relatively stable in the response phase. In contrast, sham animals exhibited metabolic responses aligned with a fasted state, with an increase in ghrelin and glucagon, and no change in GLP-1, GIP or insulin. These results suggest that gastric and esophageal neuromodulation using the neuroprosthesis may induce a state of illusory satiety and/or achieve a metabolic response commensurate with a fed, or satiated state.

### 9. Discussion

A closed-loop neuroprosthesis that restores peristalsis in models of hyper- and hypomotile pathologies was described in this example. Following sensation of a bolus, the neuroprosthesis automatically delivered electrical and chemical stimulation to generate propagative peristalsis in the esophagus. As the neuroprosthesis mechanistically recreated peristalsis by closely recapitulating neuromuscular signaling patterns, artificially generated peristalsis which approximated physiologic contractility, spatial sequence, and efficacy of propelling boluses in the esophagus. Further, in a paretic stomach, the neuroprosthesis was used to replace the enteric signaling, significantly increasing the motility rate. This is in contrast to the clinical state of art, wherein point source stimulation falls short of generating propagative peristalsis.

The neuroprosthesis could be tailored to conform to a given anatomy and pathology, given its numerous stimulation and sensing contacts and controller design. For instance, in the esophagus, the proximal third is comprised of striated muscle, whereas the distal third is comprised predominantly of smooth muscle. Based on the anatomic location of implantation, individual electrode parameters in the neuroprosthesis could be programmed to target these specific muscle types appropriately. The controller's parameters could further be adjusted to modulate the refractory period, strength, timing, sequence, and repetition of smooth muscle activation. This versatility offered therapeutic advantages for esophageal motility disorders like GERD, achalasia, jackhammer esophagus, absent peristalsis and UES/LES dysfunction, which require patient- and disease-specific realignment of neuromuscular activity involving stimulatory and inhibitory stimulation.

Furthermore, ENS neuromodulation remained underexplored due to the challenges of neural interfacing with the deep-set and distributed nature of the enteric plexi. This neuroprosthesis' design leveraged existing neural circuitry to simulate the ingestion of a meal and trigger hormonal changes, commensurate with a state of satiety. Metabolic hormones after 30 minutes of esophageal neuromodulation appeared to recapitulate a metabolic fed state signaling profile, with low glucagon and ghrelin levels. The insulin response was consistent with food consumption, even in the absence of food in this case. As such, neuromodulation could potentially produce illusory or early satiety, offering therapeutic potential for metabolic disorders like obesity, in which low or late satiety exacerbate eating tendencies. The on-demand production of insulin could also contribute to the understanding and management of type 1 diabetes and other insulin dysregulation conditions. Future research would be directed to further optimize the placement and stimulation parameters of this ENS neuroprosthetic platform.

In this study, all experiments were conducted with the animal in a fasted state. In future experiments, the neuroprosthesis may be further developed to function in a fed state and/or in a chronic setting. In addition to serving as a therapeutic device, the neuroprosthesis' electrical sensing capabilities may serve a diagnostic purpose as well. Spatially organized electrogastrograms acquired over the course of days would augment our foundational knowledge of motility. Glucagon could be utilized as a proof-of-concept inhibitory neurotransmitter to validate neuroprosthetic function. Future formulations may employ combinations of neurotransmitters informed by the evolving understanding of neural circuits governing motility. Additionally, antispasmodic drugs such as baclofen may also be used for inhibition of the smooth muscle. Future work could also explore additional functionalities of the neuroprosthesis for metabolic and immune function.

Future clinical translation of this technology may involve preclinical chronic studies in large animal models to further establish safety and efficacy, to identify disease-specific stimulation parameters, and to fine-tune the surgical techniques. This device may be useful for treating motility disorders such as gastroparesis and achalasia, and/or extend to disorders of neurochemical or metabolic signaling, such as diabetes and obesity. Temporal and neuroplastic changes in the brain, as well as habituation to stimulation may be investigated in the future. Varied stimulation paradigms and closed-loop control may present opportunities to overcome potential adaptation responses by the body. In the chronic setting, wires from the implant may be tunneled from the submucosal space to a subcutaneously-placed implantable pulse generator. Once wireless powering systems sufficiently mature, these wires could be eliminated and the prostheses could independently function in the submucosal space.

This study utilized a multifunctional, closed-loop myenteric neuroprosthesis and the SIT implantation tool, which facilitated submucosal implantation along the GI tract in an effective and safe manner through feedback-driven mucosal plane localization. The development of SIT overcame a number of the major limitations of previously reported approaches such as NOTES, including postoperative recovery, and complexity and cost of implementation. Notably, in our experiments using in the pig model, for which GI anatomy closely resembles that of a human, operators were able to perform implantation using the SIT in 75% of trials, without cautery, perforation, or trauma, illustrating the facility of use and safety. Its ability to accurately localize and safely hydrodissect the submucosa using a single tool and endoscope working channel created new opportunities for submucosal implants. The implantation and neuromodulation platform described herein thus offered an easy-to-implement, customizable and mechanism-specific approach to augment the function of the myenteric plexus for motility and metabolic disorders, thereby paving way toward bioelectronic therapies for GI dysfunction.

### 10. Material and Methods

### 10.1 Study Design

The objective was to assess the ability of the neuroprosthesis to generate coordinated patterns of motility in the esophagus and stomach under optimized electrical stimulation patterns. Assessments of the surgical complexity of implantation and biocompatibility of the implant were also performed. All large animal studies were performed in a swine model (50- to 80-kg Yorkshire pigs ranging between 4 and 6 months of age). The swine model was chosen because its gastric anatomy is similar to that of humans and has been widely used in the evaluation of biomedical GI devices. After overnight fasting, the animals were sedated with TELAZOL [tiletamine/zolazepam; 5 mg/kg, intramuscular (IM)], xylazine (2 mg/kg, IM), and atropine (0.04 mg/kg, IM) followed by endotracheal intubation and maintenance anesthesia with inhaled isoflurane (1 to 3% in oxygen) unless otherwise noted.

### 10.2 Characterization of Peristaltic Dynamics & Optimization of Electrical Stimulation Parameters

Peristalsis in response to electrical stimulation was studied using both in vivo and ex vivo models. An ex vivo tissue maintenance system was custom-made to allow the characterization of contraction dynamics under varying electrical stimulation parameters towards optimizing the dimensions, design, and control algorithm of the implant. In a bath of Krebs Ringer solution (Sigma), maintained at 37° C. using an underwater heating element, piping was constructed to allow an inflow and outflow tract for the esophagus. Measurements of tissue displacement were made possible by the use of centimeter-resolution gridding underneath the tissue and a video camera placed 3 feet above the bath. Tissue from a euthanized pig was transferred to the bath shortly upon harvest after three washes with warm phosphate buffered saline. Bipolar needle electrodes (32 g, Rythmlink) were manually inserted into the intramuscular layer at measured intervals of 1-1.5 cm along with a ground electrode.

For in vivo study, standard oval, 2.3 mm×240 cm length polypectomy snares (Telemed Systems, Inc, MA) were modified with a 25-gauge needle tip to create endoscopic intramuscular electrodes. Two to four electrodes were inserted into tissue in a sequential manner with equal spacing between electrodes (either 1 or 2 cm) in the esophagus and stomach of swine (n=5).

A range of stimulation parameters (frequencies: 20, 40, 100 Hz, amplitudes: 1-9 mA in 1 mA increments, pulse widths: 100 or 300 us, pulse train lengths: 0.5, 1, or 3 s) were pre-programmed into the Synapse system (TDT—Tucker-Davis Technologies) and output onto an IZ2 stimulator (TDT). Electrophysiological recordings were carried out on a Rz5D Base processer and PZ5 neurodigitizer amplifier (TDT) or on a RHS Stimulation/Recording System (Intan Technologies).

Endoscopic video was captured by the Pentax endoscopy suite during procedures. The peristaltic rate (number of times the pylorus closed/time) and percent closure of the muscle ring in the esophagus were used to evaluate and optimize the electrical stimulation parameters. Image analysis was performed semi-manually using functions of Fiji. Optimal spacing between contacts and depth of insertion were also evaluated to inform the design of the neuroprosthesis.

### 10.3 Neuroprosthesis Fabrication

The neuroprosthetic fiber comprises a combination of perfluoroalkoxy alkanes (PFA) coated stainless-steel (SS) electrodes, embedded in polycarbonate (PC) housing. The neuroprosthesis was fabricated via thermal drawing of a macroscale model, termed preform. To fabricate the preform, a PC rod (diameter 0.75 in; McMaster-Carr) was first machined to have a central circular channel (diameter 5.5 mm), and eight square grooves (4×4×200 mm) were machined at the periphery of the rod. Polytetrafluoroethylene (PTFE) rods were used as spacer and were placed inside the central circular channel (7/32 in, PTFE, McMaster-Carr), and peripheral groove (5/32 in, PTFE, McMaster-Carr). PC sheets (50 um, Ajedium films) were then rolled around the assembly to obtain cylinder with a diameter of 22 mm. The entire structure was then consolidated at 175° C. for 32 min under vacuum. The fiber was then draw at 270° C., and the drawing speed was varied from 0.2 to 0.4 mm/min with a feed speed of 1 mm/min to achieve draw-down ratios in the range of 14 to 20. Stainless steel wire (28G, annealed, A-M systems) coated with 330 μm of PFA was converged into the 8 outer channels of the preform while the fiber was drawn. Electrode wires from the neuroprosthesis were connectorized to 4 mm header pins (Digikey) using adhesive and flux-solder.

### 10.4 Electrode Creation

The Solafab Micromachining Tabletop workstation (Clark MXR) utilizing a Solas Ultrafast Fiber Laser was employed to etch the polymer cladding on the drawn fibers and expose electrodes contacts at the desired spacing (1-1.5 cm), determined through ex vivo peristaltic dynamics characterization. The drawn fiber was secured in a custom-made octagonal rotary jib (FIG. 22) to allow consistent positioning and etch angles for the fiber. Laser power (L20-L100), radius of etch (0.05-0.8 mm), depth (0.1-0.35 mm), length (0.25-1 cm) and pattern (repetition of strokes 1-8 times) were optimized through iterative testing to yield a flat recessed geometry for optimal current injection and contact against wet tissue. A code was used to program the Solafab computer-controlled beam and target motion system. Electrode pads were then sonicated for 15 minutes then manually cleaned under a microscope to remove residual debris following the etch. Electrodes were sterilized using ethylene oxide prior to in vivo usage.

### 10.5 Closed-Loop Electrical Stimulation Algorithm

A closed-loop control algorithm to perform stimulation in response to a change in impedance or EMG was programmed using MATLAB and the Intan RHX software (beta version, 2020). After the algorithm initialized, it began recording impedance or EMG on all stim channels. Once a threshold impedance or rectified EMG was achieved (which was dependent on the specific disease parameters), electrical stimulation was administered. Impedance was measured between a given electrode and the last electrode contact, serving as the ground electrode. The MATLAB controller interfaced with the Intan RHS Recording/Stimulation System via a Transmission Control Protocol (TCP) command interface, allowing stim parameters to be set and data to be recorded remotely.

### 10.6 Submucosal Implantation Tool Fabrication

The submucosal implantation tool was designed to perform implantation via an endoscopic approach for any devices that need placement in the submucosal cavity without the invasiveness of a NOTES or POEM technique. The tool was fabricated using a PTFE overtube (McMaster, 2.6 mm diameter). A 26 gauge needle tip was fashioned to modify a PTFE tube (2.4 mm diameter) and adhered using medical grade epoxy on a press fit design. Non-insulated 22 gauge guidewire was coated with a thin layer of silicone (Sylguard 184, Sigma) excluding the 3cm from distal tip to serve as the impedance sensing unit. Nitinol (28 gauge, Fort Wayne Metals, #8) was set in a custom vice at 400° C. for 3 minutes and quenched in water at 25° C. to create a hook. The distal tip was beveled using a dremel at an angle of 45 degrees. Following fabrication, the entire device was loaded with sterile saline and all sliding parts were articulated at least 3 times prior to use. 50 mL syringes were pre-loaded with diluted methylene blue solutions in sterile saline and connected to a syringe coupler.

### 10.7 Incisional Needle Testing

Various designs for the incising needle tip were designed in Solidworks. Mechanical tools including wire cutters, dremel, band saw, drill press, and metal sharpeners were used to fabricate the various designs. Needle tips were pneumatically secured to the descending plate of the Instron. Ex vivo esophagus and stomach tissue were held in place by vice grips forming a semi-taut interface at either 20° or 90° with respect to the axis of the needle. Force and displacement during incision were measured under a compression mode test (Bluehill Universal) which advanced the needle at 100 mm/minute.

### 10.8 Testing of Submucosal Implantation Tool Functionality

To validate the safety and efficacy of the tool, implantations were performed in swine post-euthanasia or harvested esophagus and stomach (n=5) to ensure that the tool allowed 1) incision of the mucosal layer, 2) accurate localization in the submucosal layer, 3) hydrodissection to mechanically separate the submucosal layer, and 4) no perforation of the muscular layer. During implantation, endoscopic videography was performed to visualize the process and assess the difficulty of use. Impedance was measured during the localization process using the Intan RHS Recording/Stimulation System. Following implantation, tissue cross-section was inspected for tissue damage, localization, and separation of layers. The tissue was fixed using 4% paraformaldehyde, marking the incision site with tissue marking dye (Cancer Diagnostics) and performed histology to identify whether the stated objectives were met.

### 10.9 Pressure Sensing

To characterize the pressure sensing capabilities of the neuroprosthesis, impedance from the two most proximal electrodes were monitored at 500, 1000, and 2000 Hz. Tissue in which the neuroprosthesis was implanted was explanted and placed on the Instron compression testing system, which performed controlled indentations sweeping from 0-7N, covering the range of pressures exerted by boluses of food on the equivalent surface area. Between each indentation, 0N of force were applied for 1 minute to allow the tissue to equilibrate to resting conditions.

### 10.10 Electrochemical and Mechanical Characterization of Stimulating Electrodes

Electrical impedance spectroscopy (EIS) of the stimulating electrodes was measured using a LCR meter (HP4284A, Agilent Technologies) with a sinusoidal voltage input (10 mV, 20 Hz-10 kHz). A three-electrode cell with a platinum wire as a counter electrode and a saturated Ag/AgCl reference electrode was used to perform cyclic voltammetry (CV). CV was performed in phosphate-buffered saline solution, at room temperature, using a potentiostat (Solartron, SI 1280B), and cyclic voltammetry curves were obtained at the scan rate of 20 mV/s. Charge-injection capacities were determined from voltage transient measurements in response to cathodic-first, rectangular charge-balanced biphasic pulses (100 μs, 0.5 mA to 10 mA) with a 33.3 μs interphase delay between cathodic and anodic phases using an Intan RHS Recording/Stimulation System and an oscilloscope. The maximum cathodic potential Emc was measured during the interphase interval, near-instantaneously following the ohmic voltage drop in the electrolyte (access voltage—Va). Accelerated aging test consisted of prolonged current stimulation pattern (4 mA, charge balanced biphasic rectangular pulses of half-phase period 100 μs) in a 1× PBS solution using constant current stimulators NL800A (Digitimer) for 24 h. Every 30 min to 1 h, voltage transient responses to a cathodic first biphasic stimulation with interphase delay was used to measure the E_{mc} and estimate charge injection capacity.

Cyclic buckling of the neuroprosthesis were done using a custom set-up composed of linear stage actuated by a stepper motor, described previously⁵⁰. For all samples, the neuroprosthesis was compressed then relaxed at a speed of 1 cm/sec, with a displacement of 5 mm. Bending stiffness of the fibers (n=3) were measured using a dynamical mechanical analyzer (Q800, TA Instruments). A single cantilever mode with 20 μm deformation was used within the frequency range of 0.1-200 Hz. Tensile testing of the fibers was performed at a rate of 10 mm min⁻¹ using a Zwick/Roell Z2.5 mechanical tester. The nominal stress S was measured from the recorded force divided by the cross-sectional area of the fiber, and the Young's modulus was derived from the slope of the stress-strain curve during the elastic domain. Three-point bending testing of the neuroprosthesis was performed at a rate of 60 mm/min, with a displacement of maximum 20 mm, using a Zwick/Roell Z2.5 mechanical tester. The neuroprosthesis was supported at two points which were 30 mm apart. The bending stiffness m was derived from the slope of the linear part of the force-deflection curve. The relation between the bending stiffness and the flexural strength is given by

$$m = \frac{48 {E}_{f} I}{{L}^{3}}$$

with I being the inertia moment of a cylindrical beam, E_{f} the flexural modulus, and L the support span. Knowing the inertia moment of a cylindrical beam

$$I = \frac{π {R}^{4}}{4}$$

With R radius of the fiber. We thus obtain

$${E}_{f} = \frac{{mL}^{3}}{12 π {r}^{4}}$$

### 10.11 In Vitro Assessment of Biocompatibility

An extract exposure test following ISO norms was performed to evaluate the toxicity of the neuroprosthesis material. 0.2 gm of material were added to 1 mL of DMEM culture medium and stirred at 37° C. for 24 hours or 7 days to create the extract samples. 100 uL of fetal bovine serum were added to 900 uL aliquots of the samples and used to treat C2C12 cells plated in a 96 well plate in triplicate. A negative control of untreated media and positive control of media containing 40 um MG-132, a small molecule proteasome inhibitor, were also performed. At 24 and 72 hours, cell viability was measured through quantitation of ATP using CellTiter-Glo (Promega) using a Tecan M1000Pro.

### 10.12 In Vivo Assessment of Biocompatibility

Rats (n=14, weighing between 300 and 350 g, Charles River Laboratories) were anesthetized with 1 to 2% isoflurane and premedicated with meloxicam (1 mg/kg). A small incision was made in the lateral abdomen and blunt dissection was performed to create a small subcutaneous pocket where a 1.5 cm segment of the fiber was implanted. At 2 and 4 weeks post implantation, 7 animals each were euthanized and the tissue surrounding the implant was harvested and fixed in 4% paraformaldehyde.

Additionally, a 15 cm fiber was implanted using the minimally-invasive tool in the middle esophagus and stomach of a 70 kg Yorkshire female swine under 1-2.5% inhalant anesthesia. One resolution clip (235 cm, 2.8 mm, Boston Scientific) was placed to close the incision site. No hematologic or infectious complications occurred. Seven days later, the area was examined to find no gross complications or swelling. The esophagus was excised and fixed in 4% formalin for histologic evaluation.

Following fixation, samples were washed with phosphate buffered saline (Sigma), transferred to 70% ethanol, paraffin processed and embedded. Five micron-thick sections were cut every 100 um and stained with hematoxylin and eosin.

### 10.13 Artificial Peristalsis

Following validation of the safety, efficacy, and biocompatibility, the prosthesis was implanted Yorkshire swine (n=2, 94 kg & 70 kg). The optimized electrical stimulation parameters were programmed into the controller. A pump primed the microfluidic channel and injected 5mg/mL glucagon per neurochemical stimulus.

As a model for temporarily-induced dysmotility, isoflurane anesthesia affects muscarinic receptors (TRPC4 channels, M2 and M3) and smooth muscle G-proteins as a function of concentration and anesthetic period. The following procedure was used to induce temporary dysmotility. Under 2-2.5% inhaled isoflurane anesthesia, at least three laryngeal strokes were performed and monitored through endoscopy and/or intraluminal manometry to reveal no swallow reflex. Then, barium impregnated pellets were placed in the upper, middle, and lower esophagus and monitored fluoroscopically for 10 minutes each, revealing no movement. After 1 h of anesthesia, gastric motility was depressed as evidenced by a lowered rate of peristaltic movement. As such, titrated anesthesia concentration and duration provided a suitable model for complete esophageal dysmotility and depressed gastric motility.

To characterize and validate the neuroprosthesis' ability to sense and create artificial peristalsis, a fluoroscopic pellet study was conducted. Barium sulfate (Bracco E-Z-HD Barium Sulfate for Suspension (98% w/w), Patterson Veterinary) was mixed with a sodium alginate solution (Sigma) in a 50:50 volume by weight ratio. After 10 minutes of sonication at 30 deg C., the suspension was pipetted into a custom silicone mold sized for a triple zero capsule. After 24 hours, these pellets were hydrated with a 2M CaCl2 solution for at least 2 hours and rinsed with PBS for 72 hours. They were then placed in the esophagus using the endoscope. Stimulation and sensing were performed by the neuroprosthesis and the pellet was radiographically visualized using fluoroscopy. Further, intraluminal manometry studies were performed (n=4, swine) to characterize changes in esophageal reflexes and pressures following tool-based tissue manipulation and neuroprosthesis implantation as well as capture artificial peristalsis created by the neuroprosthesis. An intraluminal manometer (Endoflip, Medtronic) was inserted into the middle and lower esophagus using an overtube to profile distensibility, pressure, pattern of motility and minimal diameter of the esophagus. Data was exported and visualized using plotting functions in Matlab. Electromyography was recorded from the electrodes during stimulation and rest periods. Data were imported and low-pass filtered to identify slow wave activity in the stomach.

### 10.14 Neuromodulation

Beyond effects on motility, neuromodulatory effects on metabolic processes was assayed by screening the blood of Yorkshire swine receiving 30 minutes of continuous esophageal muscular stimulation (n=6) or no stimulation, under sedation (n=6). A metabolic hormone panel (Eve Technologies) was run on venous blood from an ear vein catheter, collected 30 minutes prior to stimulation and every 30 minutes for 2 hours. Blood was treated with a protease inhibitor cocktail (S8830, Sigma) within 10 minutes of collection and centrifuged for 15 minutes at 4° C. at 4000 rpm. Results were normalized to the baseline values.

### 10.15 Statistical Analyses

Quantitative data are reported as mean (±standard deviation) or as a range when appropriate. The normality of the distributions was checked by the Shapiro-Wilk test. Comparative analyses were performed using student's heteroscedastic two-tailed t-test, unless otherwise noted. P<0.05 was considered significant.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using less than or equal to routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article, comprising:
a polymeric component having an aspect ratio of at least 10:1;
one or more electrodes disposed within the polymeric component, each electrode having a largest dimension aligned parallel to a largest dimension of the polymeric component; and
one or more microfluidic channels disposed within the polymeric component, each microfluidic channel having a largest dimension aligned parallel to the largest dimension of the polymeric component.

2. The article of claim 1, wherein the article is configured for implantation submucosally or intramuscularly.

3. The article of claim 1, wherein the one or more electrodes comprise sensing electrodes.

4. The article of claim 1, wherein the one or more electrodes comprise stimulating electrodes.

5. (canceled)

6. The article of claim 1, wherein each electrode comprises a conductive material, and wherein the conductive material comprises one of more selected from the group of stainless steel, gold, copper, platinum, platinum iridium, iridium oxide, a conductive fiber, and a carbon nanotube.

7. The article of claim 1, wherein the one or more electrodes are exposed to an external surrounding at predetermined locations along the polymeric component.

8. The article of claim 1, wherein the one or more electrodes are exposed to an external surrounding at a controlled spacing along the polymeric component.

9. The article of claim 8, wherein the controlled spacing is between 50 micrometers and 2 cm.

10. The article of claim 1, wherein the article comprises at least 2 electrodes disposed within the polymeric component.

11. The article of claim 1, wherein each of the one or more electrodes is exposed to an external surrounding via at least one electrode contact.

12. (canceled)

13. The article of claim 1, wherein the one or more electrodes are coated by one or more polymers.

14. (canceled)

15. The article of claim 1, wherein the polymeric component comprises polycarbonate.

16. The article of claim 1, further comprising a plurality of pressure sensors disposed along the largest dimension of the polymeric component.

17. The article of claim 1, wherein the one or more microfluidic channels contain a therapeutic agent and/or a chemical.

18. The article of claim 17, wherein the chemical comprises a neurochemical.

19. The article of claim 1, where the article has a cross-sectional dimension of between 0.5 mm and 2.5 mm.

20-23. (canceled)

24. An implantation tool, comprising:
a hollow needle comprising a tip, wherein the tip has a curved, grooved, pitchfork, broad, or dual point shape;
an overtube associated with the hollow needle, wherein the overtube is adapted and designed to receive an article configured for submucosal implantation within a lumen of a subject; and
an impedance sensor associated with the hollow needle,
wherein the incision force of the hollow needle at an angle of 20 degrees relative to the lumen is less than or equal to 5 mN.

25. The implantation tool of claim 24, further comprising a hook associated with the hollow needle.

26. The implantation tool of claim 24, wherein the tip of the hollow needle has a curved shape.

27. The implantation tool of claim 24, wherein the incision force of the hollow needle at an angle of 20 degrees relative to the lumen is less than or equal to 3 mN.

28. (canceled)

29. The implantation tool of claim 25, wherein the hook is a self-expanding hook capable of adhering onto a surface of a tissue.

30. The implantation tool of claim 25, wherein the hook comprises nitinol.

31. The implantation tool of claim 25, wherein the hook comprises a beveled tip having an angle of at least 45 degrees.

32. The implantation tool of claim 24, further comprising a guidewire capable of measuring an impedance.

33. (canceled)

34. The implantation tool of claim 24, wherein the implantation tool is configured to be inserted into an endoscope channel.

35-104. (canceled)
